SEQUENCE LISTING (149 sequences)

SEQ ID NO: 1 - protein, 15 aa, synthetic construct
  source 1..15: note = MM1 Complex 67
VSTTCWWDPPCTPNT

SEQ ID NO: 2 - protein, 9 aa, synthetic construct
  source 1..9: note = CM1
GLSGRSDDH

SEQ ID NO: 3 - protein, 5 aa, synthetic construct
  source 1..5: note = VH CDR1 Complex-67
KYAMN

SEQ ID NO: 4 - protein, 19 aa, synthetic construct
  source 1..19: note = VH CDR2 Complex-67
RIRSKYNNYATYYADSVKD

SEQ ID NO: 5 - protein, 14 aa, synthetic construct
  source 1..14: note = VH CDR3 Complex-67
HGNFGNSYISYWAY

SEQ ID NO: 6 - protein, 14 aa, synthetic construct
  source 1..14: note = VL CDR1 Complex-67
GSSTGAVTSGNYPN

SEQ ID NO: 7 - protein, 7 aa, synthetic construct
  source 1..7: note = VL CDR2 Complex 67
GTKFLAP

SEQ ID NO: 8 - protein, 9 aa, synthetic construct
  source 1..9: note = VL CDR3 Complex-67
VLWYSNRWV

SEQ ID NO: 9 - protein, 125 aa, synthetic construct
  source 1..125: note = VH1 Complex 67
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 10 - protein, 109 aa, synthetic construct
  source 1..109: note = VL1 Complex-67
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 11 - protein, 249 aa, synthetic construct
  source 1..249: note = scFv Complex-67
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 12 - protein, 10 aa, synthetic construct
  source 1..10: note = Linker
GSSGGSGGSG

SEQ ID NO: 13 - protein, 15 aa, synthetic construct
  source 1..15: note = MM2 Complex-67 Complex-57
LSCEGWAMNREQCRA

SEQ ID NO: 14 - protein, 13 aa, synthetic construct
  source 1..13: note = CM2
ISSGLLSGRSDQH

SEQ ID NO: 15 - protein, 5 aa, synthetic construct
  source 1..5: note = VH2 CDR1 Complex-67 Complex-57
NYGVH

SEQ ID NO: 16 - protein, 16 aa, synthetic construct
  source 1..16: note = VH2 CDR3 Complex-67 Complex-57
VIWSGGNTDYNTPFTS

SEQ ID NO: 17 - protein, 11 aa, synthetic construct
  source 1..11: note = VH2 CDR3 Complex 67 Complex 57
ALTYYDYEFAY

SEQ ID NO: 18 - protein, 11 aa, synthetic construct
  source 1..11: note = VL2 CDR1 Complex 67 Complex 57
RASQSIGTNIH

SEQ ID NO: 19 - protein, 7 aa, synthetic construct
  source 1..7: note = VL2 CDR2 Complex-67 Complex-57
YASESIS

SEQ ID NO: 20 - protein, 9 aa, synthetic construct
  source 1..9: note = VL2 CDR3 Complex 67 Complex 57
QQNNNWPTT

SEQ ID NO: 21 - protein, 119 aa, synthetic construct
  source 1..119: note = VH2 Domain Complex-67 Complex-57 CI106 (Control)
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSA

SEQ ID NO: 22 - protein, 107 aa, synthetic construct
  source 1..107: note = VL2 Domain Complex-67 Complex-57 CI106 (Control)
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 23 - protein, 217 aa, synthetic construct
  source 1..217: note = Fc1 Complex-67 Complex-57
PAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 24 - protein, 218 aa, synthetic construct
  source 1..218: note = Fc1 with terminal lysine
PAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 25 - protein, 107 aa, synthetic construct [sequence omitted]

SEQ ID NO: 26 - protein, 98 aa, synthetic construct
  source 1..98: note = CH1 Human IgG1 Complex 67 Complex 57
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 27 - skipped

SEQ ID NO: 28 - protein, 217 aa, synthetic construct
  source 1..217: note = Fc2 w/o C-terminal lysine Complex-67 Complex-57
PAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 29 - protein, 218 aa, synthetic construct
  source 1..218: note = Fc2 with C-terminal lysine
PAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 30 - protein, 746 aa, synthetic construct
  source 1..746: note = First Polypeptide Complex -67
QGQSGSVSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 31 - protein, 261 aa, synthetic construct
  source 1..261: note = Second Polypeptide Complex 67 Complex 57 CI106 control
QGQSGQGLSCEGWAMNREQCRAGGGSSGGSISSGLLSGRSDQHGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 226 aa, synthetic construct
  source 1..226: note = Third Polypeptide Complex 67 Complex 57
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 33 - protein, 6 aa, synthetic construct
  source 1..6: note = Spacer
QGQSGS

SEQ ID NO: 34 - protein, 14 aa, synthetic construct
  source 1..14: note = Hinge-1 Complex-67 Complex-57 CI106 (control)
EPKSCDKTHTCPPC

SEQ ID NO: 35 - protein, 9 aa, synthetic construct
  source 1..9: note = Hinge-2 Complex-67 Complex-57
DKTHTCPPC

SEQ ID NO: 36 - protein, 227 aa, synthetic construct
  source 1..227: note = Third Polypeptide with terminal lysine Complex-67 Complex-57
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 37 - protein, 254 aa, synthetic construct
  source 1..254: note = Complex 67 Complex 57 2nd polypeptide without spacer
LSCEGWAMNREQCRAGGGSSGGSISSGLLSGRSDQHGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 746 aa, synthetic construct
  source 1..746: note = Complex-57 1st Polypeptide w/o terminal lysine.
QGQSGSGYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 39 - protein, 4 aa, synthetic construct
  source 1..4: note = Linker
GGGS

SEQ ID NO: 40 - protein, 4 aa, synthetic construct
  source 1..4: note = Linker
  REGION >4: note = the entire sequence of amino acids can be repeated one or more times.
GGGS

SEQ ID NO: 41 - protein, 5 aa, synthetic construct
  source 1..5: note = Linker
  REGION >5: note = the entire sequence of amino acids can be repeated one or more times.
GSGGS

SEQ ID NO: 42 - protein, 4 aa, synthetic construct
  source 1..4: note = Linker
GGSG

SEQ ID NO: 43 - protein, 5 aa, synthetic construct
  source 1..5: note = Linker
GGSGG

SEQ ID NO: 44 - protein, 5 aa, synthetic construct
  source 1..5: note = Linker
GSGSG

SEQ ID NO: 45 - protein, 5 aa, synthetic construct
  source 1..5: note = Linker
GSGGG

SEQ ID NO: 46 - protein, 5 aa, synthetic construct
  source 1..5: note = Linker
GGGSG

SEQ ID NO: 47 - protein, 5 aa, synthetic construct
  source 1..5: note = Linker
GSSSG

SEQ ID NO: 48 - protein, 17 aa, synthetic construct
  source 1..17: note = Linker
GGGGSGGGGSGGGGSGS

SEQ ID NO: 49 - protein, 7 aa, synthetic construct
  source 1..7: note = Linker
GGGGSGS

SEQ ID NO: 50 - protein, 15 aa, synthetic construct
  source 1..15: note = Linker
GGGGSGGGGSGGGGS

SEQ ID NO: 51 - protein, 20 aa, synthetic construct
  source 1..20: note = Linker
GGGGSGGGGSGGGGSGGGGS

SEQ ID NO: 52 - protein, 5 aa, synthetic construct
  source 1..5: note = Linker
GGGGS

SEQ ID NO: 53 - protein, 10 aa, synthetic construct
  source 1..10: note = Linker
GGGGSGGGGS

SEQ ID NO: 54 - protein, 4 aa, synthetic construct
  source 1..4: note = Linker
GGGS

SEQ ID NO: 55 - protein, 8 aa, synthetic construct
  source 1..8: note = Linker
GGGSGGGS

SEQ ID NO: 56 - protein, 12 aa, synthetic construct
  source 1..12: note = Linker
GGGSGGGSGGGS

SEQ ID NO: 57 - protein, 11 aa, synthetic construct
  source 1..11: note = Linker
GSSGGSGGSGG

SEQ ID NO: 58 - protein, 24 aa, synthetic construct
  source 1..24: note = Linker
GGGSGGGGSGGGGSGGGGSGGGGS

SEQ ID NO: 59 - protein, 16 aa, synthetic construct
  source 1..16: note = Linker
GSTSGSGKPGSSEGST

SEQ ID NO: 60 - protein, 17 aa, synthetic construct
  source 1..17: note = Linker
SKYGPPCPPCPAPEFLG

SEQ ID NO: 61 - protein, 12 aa, synthetic construct
  source 1..12: note = Linker
GGSLDPKGGGGS

SEQ ID NO: 62 - protein, 20 aa, synthetic construct
  source 1..20: note = Linker
PKSCDKTHTCPPCPAPELLG

SEQ ID NO: 63 - protein, 12 aa, synthetic construct
  source 1..12: note = Linker
GKSSGSGSESKS

SEQ ID NO: 64 - protein, 14 aa, synthetic construct
  source 1..14: note = Linker
GSTSGSGKSSEGKG

SEQ ID NO: 65 - protein, 18 aa, synthetic construct
  source 1..18: note = Linker
GSTSGSGKSSEGSGSTKG

SEQ ID NO: 66 - protein, 18 aa, synthetic construct
  source 1..18: note = Linker
GSTSGSGKPGSGEGSTKG

SEQ ID NO: 67 - protein, 15 aa, synthetic construct
  source 1..15: note = MM1
MMYCGGNEVLCGPRV

SEQ ID NO: 68 - protein, 15 aa, synthetic construct
  source 1..15: note = MM1
GYRWGCEWNCGGITT

SEQ ID NO: 69 - protein, 15 aa, synthetic construct
  source 1..15: note = mm1
MMYCGGNEIFCEPRG

SEQ ID NO: 70 - protein, 15 aa, synthetic construct
  source 1..15: note = MM1
GYGWGCEWNCGGSSP

SEQ ID NO: 71 - protein, 15 aa, synthetic construct
  source 1..15: note = MM1
MMYCGGNEIFCGPRG

SEQ ID NO: 72 - protein, 15 aa, synthetic construct
  source 1..15: note = MM1
GYLWGCEWNCGGITT

SEQ ID NO: 73 - protein, 8 aa, synthetic construct
  source 1..8: note = CM1 Complex-67 Complex-57 CI106
LSGRSDDH

SEQ ID NO: 74 - protein, 13 aa, synthetic construct
  source 1..13: note = CM
ISSGLLSGRSDQH

SEQ ID NO: 75 - protein, 8 aa, synthetic construct
  source 1..8: note = CM
LSGRSDNH

SEQ ID NO: 76 - protein, 12 aa, synthetic construct
  source 1..12: note = CM
TSTSGRSANPRG

SEQ ID NO: 77 - protein, 10 aa, synthetic construct
  source 1..10: note = CM
VHMPLGFLGP

SEQ ID NO: 78 - protein, 8 aa, synthetic construct
  source 1..8: note = CM
AVGLLAPP

SEQ ID NO: 79 - protein, 8 aa, synthetic construct
  source 1..8: note = CM
QNQALRMA

SEQ ID NO: 80 - protein, 8 aa, synthetic construct
  source 1..8: note = CM
ISSGLLSS

SEQ ID NO: 81 - protein, 13 aa, synthetic construct
  source 1..13: note = CM
ISSGLLSGRSDNH

SEQ ID NO: 82 - protein, 8 aa, synthetic construct
  source 1..8: note = CM
LSGRSGNH

SEQ ID NO: 83 - protein, 8 aa, synthetic construct
  source 1..8: note = CM
LSGRSDIH

SEQ ID NO: 84 - protein, 8 aa, synthetic construct
  source 1..8: note = CM
LSGRSDQH

SEQ ID NO: 85 - protein, 8 aa, synthetic construct
  source 1..8: note = CM
LSGRSDTH

SEQ ID NO: 86 - protein, 8 aa, synthetic construct
  source 1..8: note = CM
LSGRSDYH

SEQ ID NO: 87 - protein, 8 aa, synthetic construct
  source 1..8: note = CM
LSGRSDNP

SEQ ID NO: 88 - protein, 8 aa, synthetic construct
  source 1..8: note = CM
LSGRSANP

SEQ ID NO: 89 - protein, 8 aa, synthetic construct
  source 1..8: note = CM
LSGRSANI

SEQ ID NO: 90 - protein, 8 aa, synthetic construct
  source 1..8: note = CM
LSGRSDNI

SEQ ID NO: 91 - protein, 15 aa, synthetic construct
  source 1..15: note = CM
ISSGLLSGRSANPRG

SEQ ID NO: 92 - protein, 18 aa, synthetic construct
  source 1..18: note = CM
AVGLLAPPTSGRSANPRG

SEQ ID NO: 93 - protein, 17 aa, synthetic construct
  source 1..17: note = CM
AVGLLAPPSGRSANPRG

SEQ ID NO: 94 - protein, 13 aa, synthetic construct
  source 1..13: note = CM
ISSGLLSGRSDDH

SEQ ID NO: 95 - protein, 13 aa, synthetic construct
  source 1..13: note = CM
ISSGLLSGRSDIH

SEQ ID NO: 96 - protein, 13 aa, synthetic construct
  source 1..13: note = CM
ISSGLLSGRSDTH

SEQ ID NO: 97 - protein, 13 aa, synthetic construct
  source 1..13: note = CM
ISSGLLSGRSDYH

SEQ ID NO: 98 - protein, 13 aa, synthetic construct
  source 1..13: note = CM
ISSGLLSGRSDNP

SEQ ID NO: 99 - protein, 13 aa, synthetic construct
  source 1..13: note = CM
ISSGLLSGRSANP

SEQ ID NO: 100 - protein, 13 aa, synthetic construct
  source 1..13: note = CM
ISSGLLSGRSANI

SEQ ID NO: 101 - protein, 18 aa, synthetic construct
  source 1..18: note = CM
AVGLLAPPGGLSGRSDDH

SEQ ID NO: 102 - protein, 18 aa, synthetic construct
  source 1..18: note = CM
AVGLLAPPGGLSGRSDIH

SEQ ID NO: 103 - protein, 18 aa, synthetic construct
  source 1..18: note = CM
AVGLLAPPGGLSGRSDQH

SEQ ID NO: 104 - protein, 18 aa, synthetic construct
  source 1..18: note = CM
AVGLLAPPGGLSGRSDTH

SEQ ID NO: 105 - protein, 18 aa, synthetic construct
  source 1..18: note = CM
AVGLLAPPGGLSGRSDYH

SEQ ID NO: 106 - protein, 18 aa, synthetic construct
  source 1..18: note = CM
AVGLLAPPGGLSGRSDNP

SEQ ID NO: 107 - protein, 18 aa, synthetic construct
  source 1..18: note = CM
AVGLLAPPGGLSGRSANP

SEQ ID NO: 108 - protein, 18 aa, synthetic construct
  source 1..18: note = CM
AVGLLAPPGGLSGRSANI

SEQ ID NO: 109 - protein, 13 aa, synthetic construct
  source 1..13: note = CM
ISSGLLSGRSDNI

SEQ ID NO: 110 - protein, 18 aa, synthetic construct
  source 1..18: note = CM
AVGLLAPPGGLSGRSDNI

SEQ ID NO: 111 - protein, 13 aa, synthetic construct
  source 1..13: note = CM
ISSGLLSGRSGNH

SEQ ID NO: 112 - DNA, 2241 bp, synthetic construct
  source 1..2241: mol_type = other DNA; note = Complex-67 Polynucleotide Encoding a First Polypeptide
caaggacaatctggctctgtgtccaccacctgttggtgggaccctccatgcacacctaataccggcagctctggtggctctggcggaagcggaggactgtctggcagatccgatgatcacggcggaggatctgaggtgcagctggttgaatctggtggcggactggttcagcctggcggatctctgaaactgagctgtgccgccagcggcttcaccttcaacaaatacgccatgaactgggtccgacaggcccctggcaaaggccttgaatgggtcgccagaatcagaagcaagtacaacaactatgccacctactacgccgacagcgtgaaggacagattcaccatcagccgggacgacagcaagaacaccgcctacctgcagatgaacaacctgaaaaccgaggacaccgccgtgtactactgtgtgcggcacggcaacttcggcaacagctacatcagctactgggcctattggggccagggcacactggtcacagtttctagtggcggaggcggatctggcggcggtggaagtggcggcggaggttctcaaacagtggtcacccaagagcctagcctgaccgtttctcctggcggaaccgtgacactgacatgcggatcttctacaggcgccgtgaccagcggcaactaccctaattgggtgcagcagaagccaggccaggctcctagaggactgatcggcggcacaaagtttctggctcccggaacaccagccagattcagcggttctctgctcggaggaaaggccgctctgacactttctggcgtgcagcctgaggatgaggccgagtactattgcgtgctgtggtacagcaacagatgggtgttcggcggaggcaccaagctgacagttcttggaggtggcggtagccaggtccagctgaaacaatctggacccggactcgtgcagccaagccagagcctgtctatcacctgtaccgtgtccggcttcagcctgaccaattacggcgtgcactgggttcgacaatctcccggcaagggactcgaatggctgggagtgatttggagcggcggcaacaccgactacaacaccccattcaccagcagactgagcatcaacaaggacaacagcaagtcccaggtgttcttcaagatgaactccctgcagagccaggataccgccatctattactgcgctcgggccctgacctactatgactacgagtttgcctactggggacagggaaccctcgtgacagtgtctgctgctagcacaaagggccctagcgttttcccactggctcccagcagcaagtctacatccggtggaacagccgctctgggctgcctggtcaaggattactttcccgagccagtgaccgtgtcctggaatagcggagcactgacatctggcgtgcacacatttccagccgtgctgcagtctagcggcctgtactctctgtccagcgttgtgacagtgcccagcagctctctgggcacccagacctacatctgcaatgtgaaccacaagcctagcaacaccaaggtggacaagaaggtggaacccaagagctgcgataagacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagttcaattggtacgtcgacggcgtggaagtgcacaatgccaagaccaagccttgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccggaaagagatgaccaagaatcaggtgtccctgacctgcctggtcaagggcttctacccttccgatatcgccgtggaatgggagagcaatggacagcccgagaacaactacaagacaacccctcctgtgctgaagtccgacggctcattcttcctgtacagcaagctgaccgtggacaagagcagatggcagcagggcaacgtgttcagctgcagcgtgatgcacgaggccctgcacaaccactacacccagaagtccctgtctctgagccccggcaaa

SEQ ID NO: 113 - DNA, 783 bp, synthetic construct
  source 1..783: mol_type = other DNA; note = Polynucleotide Encoding a Second Polypeptide
caaggccagtctggccaaggtcttagttgtgaaggttgggcgatgaatagagaacaatgtcgagccggaggtggctcgagcggcggctctatctcttccggactgctgtccggcagatccgaccagcacggcggaggatcccaaatcctgctgacacagtctcctgtcatactgagtgtctcccccggcgagagagtctctttctcatgtcgggccagtcagtctattgggactaacatacactggtaccagcaacgcaccaacggaagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 114 - DNA, 681 bp, synthetic construct
  source 1..681: mol_type = other DNA; note = Polynucleotide Encoding a Third Polypeptide
gataagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagacaaagccctgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccgggaagagatgaccaagaaccaggtgtccctgacctgcctggtcaagggcttctacccttccgatatcgccgtggaatgggagagcaatggacagcccgagaacaactacgacaccacacctccagtgctggacagcgacggctcattcttcctgtacagcgacctgaccgtggacaagagcagatggcagcagggcaacgtgttcagctgcagcgtgatgcacgaggccctgcacaaccactacacccagaagtccctgagcctgtctcctggcaaa

SEQ ID NO: 115 - DNA, 783 bp, synthetic construct
  source 1..783: mol_type = other DNA; note = Light Chain
caaggacaatctggacagggcctgagctgtgaaggctgggccatgaatagagagcagtgcagagctggcggcggatcttctggcggctctatctcttctggactgctgagcggcagaagcgatcaacacggcggaggctctcagatcctgctgacacagagccccgtgatcctgtctgtgtctcctggcgagagagtgtccttcagctgtagagccagccagtccatcggcaccaacatccactggtatcagcagcggaccaacggcagccccagactgctgattaagtacgccagcgagagcatcagcggcatccccagcagattttctggcagcggctctggcaccgacttcaccctgagcatcaacagcgtggaaagcgaggatatcgccgactactactgccagcagaacaacaactggcccaccacctttggagccggcaccaagctggaactgaagagaacagtggccgctcctagcgtgttcatcttcccaccttccgacgagcagctgaaaagcggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctacagcctgagcagcaccctgacactgagcaaggccgactacgagaagcacaaagtgtacgcctgcgaagtgacccaccagggcctttctagccctgtgaccaagagcttcaaccggggcgagtgt

SEQ ID NO: 116 - protein, 6 aa, synthetic construct
  source 1..6: note = spacer
QGQSGS

SEQ ID NO: 117 - protein, 7 aa, synthetic construct
  source 1..7: note = spacer
QGQSGQG

SEQ ID NO: 118 - protein, 6 aa, synthetic construct
  source 1..6: note = spacer
QGQSGS

SEQ ID NO: 119 - protein, 7 aa, synthetic construct
  source 1..7: note = spacer
QGQSGQG

SEQ ID NO: 120 - protein, 741 aa, synthetic construct
  source 1..741: note = First polypeptide without spacer
VSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 121 - skipped

SEQ ID NO: 122 - protein, 249 aa, synthetic construct
  source 1..249: note = Anti-CD3 scFv V16 Complex-57 CI106
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 123 - protein, 747 aa, synthetic construct
  source 1..747: note = CI106 - Heavy Chain CRF41-2008C225v5Fcmt4h20GG-0011v16sc-H-N
QGQSGSGYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 218 aa, synthetic construct
  source 1..218: note = Fc CI106
PAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 2241 bp, synthetic construct
  source 1..2241: mol_type = other DNA; note = CI106 Heavy Chain - CRF41-2008C225v5Fcmt4h20GG-0011v16sc-H-N
caaggccagtctggatccggttatctgtggggttgcgagtggaattgcggagggatcactacaggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 126 - protein, 5 aa, synthetic construct
  source 1..5: note = Linker
  REGION >5: note = wherein the amino acid sequence can be repeated between 1 and 10 times.
GGGGS

SEQ ID NO: 127 - protein, 8 aa, synthetic construct
  source 1..8: note = Linker
GGGSSGGS

SEQ ID NO: 128 - protein, 5 aa, synthetic construct
  source 1..5: note = VH1 CDR1 Complex-57 CI106 (control)
TYAMN

SEQ ID NO: 129 - protein, 19 aa, synthetic construct
  source 1..19: note = VH1 CDR2 Complex-57 CI106 (control)
RIRSKYNNYATYYADSVKD

SEQ ID NO: 130 - protein, 14 aa, synthetic construct
  source 1..14: note = VH1 CDR3 Complex-57 CI106 (control)
HGNFGNSYVSWFAY

SEQ ID NO: 131 - protein, 14 aa, synthetic construct
  source 1..14: note = VL1 CDR1 Complex-57 CI106 (control)
RSSTGAVTTSNYAN

SEQ ID NO: 132 - protein, 7 aa, synthetic construct
  source 1..7: note = VL1 CDR2 Complex-57 CI106 (control)
GTNKRAP

SEQ ID NO: 133 - protein, 9 aa, synthetic construct
  source 1..9: note = VL1 CDR3 Complex-57 CI106 (control)
ALWYSNLWV

SEQ ID NO: 134 - protein, 125 aa, synthetic construct
  source 1..125: note = VH1 Domain Complex-57 CI106 (control)
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 135 - protein, 109 aa, synthetic construct
  source 1..109: note = VL1 Domain Complex-57 CI106 (control)
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVL

SEQ ID NO: 136 - protein, 747 aa, synthetic construct
  source 1..747: note = Complex-57 First Polypeptide with terminal lysine
QGQSGSGYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 137 - protein, 747 aa, synthetic construct
  source 1..747: note = First Polypeptide Complex-67 w/terminal lysine
QGQSGSVSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 138 - skipped

SEQ ID NO: 139 - DNA, 2238 bp, synthetic construct
  source 1..2238: mol_type = other DNA; note = Polynucleotide encoding 1st polypeptide w/o terminal lysine Complex-67
caaggacaatctggctctgtgtccaccacctgttggtgggaccctccatgcacacctaataccggcagctctggtggctctggcggaagcggaggactgtctggcagatccgatgatcacggcggaggatctgaggtgcagctggttgaatctggtggcggactggttcagcctggcggatctctgaaactgagctgtgccgccagcggcttcaccttcaacaaatacgccatgaactgggtccgacaggcccctggcaaaggccttgaatgggtcgccagaatcagaagcaagtacaacaactatgccacctactacgccgacagcgtgaaggacagattcaccatcagccgggacgacagcaagaacaccgcctacctgcagatgaacaacctgaaaaccgaggacaccgccgtgtactactgtgtgcggcacggcaacttcggcaacagctacatcagctactgggcctattggggccagggcacactggtcacagtttctagtggcggaggcggatctggcggcggtggaagtggcggcggaggttctcaaacagtggtcacccaagagcctagcctgaccgtttctcctggcggaaccgtgacactgacatgcggatcttctacaggcgccgtgaccagcggcaactaccctaattgggtgcagcagaagccaggccaggctcctagaggactgatcggcggcacaaagtttctggctcccggaacaccagccagattcagcggttctctgctcggaggaaaggccgctctgacactttctggcgtgcagcctgaggatgaggccgagtactattgcgtgctgtggtacagcaacagatgggtgttcggcggaggcaccaagctgacagttcttggaggtggcggtagccaggtccagctgaaacaatctggacccggactcgtgcagccaagccagagcctgtctatcacctgtaccgtgtccggcttcagcctgaccaattacggcgtgcactgggttcgacaatctcccggcaagggactcgaatggctgggagtgatttggagcggcggcaacaccgactacaacaccccattcaccagcagactgagcatcaacaaggacaacagcaagtcccaggtgttcttcaagatgaactccctgcagagccaggataccgccatctattactgcgctcgggccctgacctactatgactacgagtttgcctactggggacagggaaccctcgtgacagtgtctgctgctagcacaaagggccctagcgttttcccactggctcccagcagcaagtctacatccggtggaacagccgctctgggctgcctggtcaaggattactttcccgagccagtgaccgtgtcctggaatagcggagcactgacatctggcgtgcacacatttccagccgtgctgcagtctagcggcctgtactctctgtccagcgttgtgacagtgcccagcagctctctgggcacccagacctacatctgcaatgtgaaccacaagcctagcaacaccaaggtggacaagaaggtggaacccaagagctgcgataagacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagttcaattggtacgtcgacggcgtggaagtgcacaatgccaagaccaagccttgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccggaaagagatgaccaagaatcaggtgtccctgacctgcctggtcaagggcttctacccttccgatatcgccgtggaatgggagagcaatggacagcccgagaacaactacaagacaacccctcctgtgctgaagtccgacggctcattcttcctgtacagcaagctgaccgtggacaagagcagatggcagcagggcaacgtgttcagctgcagcgtgatgcacgaggccctgcacaaccactacacccagaagtccctgtctctgagccccggc

SEQ ID NO: 140 - protein, 227 aa, synthetic construct; identical to SEQ ID NO: 36
  source 1..227: note = 3rd Polypeptide w/C-terminal lysine Complex-67 Complex-57

SEQ ID NO: 141 - DNA, 678 bp, synthetic construct
  source 1..678: mol_type = other DNA; note = Polynucleotide encoding 3rd Polypeptide without codon encoding Cterminal lysine Complex-67 Complex-57
gataagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagacaaagccctgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccgggaagagatgaccaagaaccaggtgtccctgacctgcctggtcaagggcttctacccttccgatatcgccgtggaatgggagagcaatggacagcccgagaacaactacgacaccacacctccagtgctggacagcgacggctcattcttcctgtacagcgacctgaccgtggacaagagcagatggcagcagggcaacgtgttcagctgcagcgtgatgcacgaggccctgcacaaccactacacccagaagtccctgagcctgtctcctggc

SEQ ID NO: 142 - DNA, 2238 bp, synthetic construct
  source 1..2238: mol_type = other DNA; note = Polynucleotide encoding 1st polypeptide (without codon for C-terminal lysine) Complex-57
caaggacaatctggatccggctatctgtggggctgcgagtggaattgtggcggcatcacaacaggctctagcggcggaagcggaggatctggtggactgtctggcagatccgatgatcatggcggcggatcccagaccgtggtcacacaagagcctagcttctccgtgtctcctggcggcacagtgaccctgacatgcagatcttctacaggcgccgtgaccaccagcaactacgccaattgggtgcagcagacccctggacaggctcctagaggactgatcggcggcaccaacaaaagagcccctggcgtcccagatagattcagcggctctatcctgggcaacaaggccgcactgacaatcacaggcgcccaggccgatgacgagagcgattactattgcgccctgtggtacagcaacctgtgggttttcggcggaggcaccaagctgacagttcttggcggaggcggaagtggtggtggcggatctggtggcggtggatctgaagtgcagctggtggaatctggcggaggacttgttcagccaggcggctctctgaagctgtcttgtgccgcctccggcttcacctttagcacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagaacaccgcctacctgcagatgaacagcctgaaaaccgaggacaccgccgtgtactactgcaccagacacggcaacttcggcaacagctatgtgtcttggtttgcctactggggccagggcacactggtcacagttagttctggcggcggaggttctcaggtgcagctgaaacagtctggccctggactggtgcagcctagccagtctctgagcatcacctgtaccgtgtccggcttctccctgaccaattacggcgtgcactgggttcgacaatccccaggcaagggactcgaatggctgggagtgatttggagcggcggcaacaccgactacaacaccccattcaccagcagactgtccatcaacaaggacaacagcaagtcccaggtgttcttcaagatgaactccctgcagagccaggataccgccatctattactgcgctcgggccctgacctactatgactacgagttcgcctattggggacagggaaccctcgtgacagtgtctgccgctagcacaaagggccctagcgttttcccactggctcccagcagcaagtctacatccggtggaacagccgctctgggctgcctggtcaaggattactttcccgagccagtgaccgtgtcctggaatagcggagcactgacatctggcgtgcacacatttccagccgtgctgcagtctagcggcctgtactctctgtccagcgttgtgacagtgcccagcagctctctgggcacccagacctacatctgcaatgtgaaccacaagcctagcaacaccaaggtggacaagaaggtggaacccaagagctgcgataagacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagttcaattggtacgtcgacggcgtggaagtgcacaatgccaagaccaagccttgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccggaaagagatgaccaagaatcaggtgtccctgacctgcctggtcaagggcttctacccttccgatatcgccgtggaatgggagagcaatggacagcccgagaacaactacaagacaacccctcctgtgctgaagtccgacggctcattcttcctgtacagcaagctgaccgtggacaagagcagatggcagcagggcaacgtgttcagctgcagcgtgatgcacgaggccctgcacaaccactacacccagaagtccctgtctctgagccccggc

SEQ ID NO: 143 - DNA, 2241 bp, synthetic construct
  source 1..2241: mol_type = other DNA; note = Polynucleotide encoding 1st polypeptide (with codon for C-terminal lysine) Complex-57
caaggacaatctggatccggctatctgtggggctgcgagtggaattgtggcggcatcacaacaggctctagcggcggaagcggaggatctggtggactgtctggcagatccgatgatcatggcggcggatcccagaccgtggtcacacaagagcctagcttctccgtgtctcctggcggcacagtgaccctgacatgcagatcttctacaggcgccgtgaccaccagcaactacgccaattgggtgcagcagacccctggacaggctcctagaggactgatcggcggcaccaacaaaagagcccctggcgtcccagatagattcagcggctctatcctgggcaacaaggccgcactgacaatcacaggcgcccaggccgatgacgagagcgattactattgcgccctgtggtacagcaacctgtgggttttcggcggaggcaccaagctgacagttcttggcggaggcggaagtggtggtggcggatctggtggcggtggatctgaagtgcagctggtggaatctggcggaggacttgttcagccaggcggctctctgaagctgtcttgtgccgcctccggcttcacctttagcacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagaacaccgcctacctgcagatgaacagcctgaaaaccgaggacaccgccgtgtactactgcaccagacacggcaacttcggcaacagctatgtgtcttggtttgcctactggggccagggcacactggtcacagttagttctggcggcggaggttctcaggtgcagctgaaacagtctggccctggactggtgcagcctagccagtctctgagcatcacctgtaccgtgtccggcttctccctgaccaattacggcgtgcactgggttcgacaatccccaggcaagggactcgaatggctgggagtgatttggagcggcggcaacaccgactacaacaccccattcaccagcagactgtccatcaacaaggacaacagcaagtcccaggtgttcttcaagatgaactccctgcagagccaggataccgccatctattactgcgctcgggccctgacctactatgactacgagttcgcctattggggacagggaaccctcgtgacagtgtctgccgctagcacaaagggccctagcgttttcccactggctcccagcagcaagtctacatccggtggaacagccgctctgggctgcctggtcaaggattactttcccgagccagtgaccgtgtcctggaatagcggagcactgacatctggcgtgcacacatttccagccgtgctgcagtctagcggcctgtactctctgtccagcgttgtgacagtgcccagcagctctctgggcacccagacctacatctgcaatgtgaaccacaagcctagcaacaccaaggtggacaagaaggtggaacccaagagctgcgataagacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagttcaattggtacgtcgacggcgtggaagtgcacaatgccaagaccaagccttgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccggaaagagatgaccaagaatcaggtgtccctgacctgcctggtcaagggcttctacccttccgatatcgccgtggaatgggagagcaatggacagcccgagaacaactacaagacaacccctcctgtgctgaagtccgacggctcattcttcctgtacagcaagctgaccgtggacaagagcagatggcagcagggcaacgtgttcagctgcagcgtgatgcacgaggccctgcacaaccactacacccagaagtccctgtctctgagccccggcaaa

SEQ ID NO: 144 - protein, 740 aa, synthetic construct
  source 1..740: note = Complex-67 1st polypeptide without the spacer and without a terminal lysine
VSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 145 - protein, 740 aa, synthetic construct
  source 1..740: note = Complex-57 1st Polypeptide without the spacer and without a terminal lysine
GYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 146 - protein, 7 aa, synthetic construct
  source 1..7: note = CM
ALAHGLF

SEQ ID NO: 147 - protein, 11 aa, synthetic construct
  source 1..11: note = CM
APRSALAHGLF

SEQ ID NO: 148 - protein, 12 aa, synthetic construct
  source 1..12: note = CM
ISSGLLSGRSNI

SEQ ID NO: 149 - protein, 7 aa, synthetic construct
  source 1..7: note = CM
LSGRSNI

(19) United States
(12) Patent Application Publication
(10) Pub. No.: US 2023/0183382 A1
(43) Pub. Date: Jun. 15, 2023

(54) ACTIVATABLE POLYPEPTIDE COMPLEX

(71) Applicants: CytomX Therapeutics, Inc., South San Francisco, CA (US); Amgen, Inc., Thousand Oaks, CA (US)

(72) Inventors: Leila M. BOUSTANY, Redwood City, CA (US); Madan M. PAIDHUNGAT, San Francisco, CA (US); Ellaine Anne Mariano FOX, San Francisco, CA (US); Sayantan MITRA, Mountain View, CA (US); W. Michael KAVANAUGH, South San Francisco, CA (US); Raffaella BRIANTE, South San Francisco, CA (US); Jennitte LeAnn STEVENS, Thousand Oaks, CA (US)

(73) Assignees: CytomX Therapeutics, Inc., South San Francisco, CA (US); Amgen, Inc., Thousand Oaks, CA (US)

(21) Appl. No.: 18/046,855

(22) Filed: Oct. 14, 2022

Related U.S. Application Data
(60) Provisional application No. 63/370,895, filed on Aug. 9, 2022.
(60) Provisional application No. 63/256,410, filed on Oct. 15, 2021.

(51) Int. Cl. C07K 16/46; C07K 16/28

(52) U.S. Cl. CPC C07K 16/468; C07K 16/2863; C07K 16/2809; C07K 2317/31; C07K 2317/52; C07K 2317/565; A61K 2039/505

(57) ABSTRACT

The present disclosure relates to activatable anti-EGFR, anti-CD3, heteromultimeric bispecific polypeptide complexes (HBPCs) and methods of making and using the same.

### CROSS-REFERENCE TO RELATED APPLICATIONS

This application claims the priority benefit of U.S. Provisional Application Nos. 63/256,410, filed Oct. 15, 2021, and 63/370,895, filed Aug. 9, 2022, which are incorporated herein by reference in their entireties.

### REFERENCE TO SEQUENCE LISTING SUBMITTED ELECTRONICALLY VIA EFS WEB

The content of the electronically submitted sequence listing (4681_001PC02_Seqlisting_ST26.xml; Size: 179,444 bytes; and Date of Creation: Oct. 13, 2022) submitted in this application is incorporated herein by reference in its entirety.

### FIELD

The present disclosure relates to activatable anti-EGFR, anti-CD3, heteromultimeric bispecific polypeptide complexes (HBPCs) and methods of making and using the same.

### BACKGROUND

The generation and activation of tumor antigen-specific T cells are involved in immune-mediated control of development and anti-tumor activity. This requires multiple T-cell co-stimulatory receptors and T-cell negative regulators, or co-inhibitory receptors, acting in concert to control T-cell activation, proliferation, and gain or loss of effector function. Tumor-specific T-cell responses are difficult to mount and sustain in cancer patients, due to the numerous immune escape mechanisms of tumor cells. However, attempts have been made to harness T cells for cancer therapies. Such approaches include using T cell engaging bispecific antibodies which bind both a surface target antigen on a cancer cell, and a T cell surface antigen, such as CD3, on T cells. Generally, by binding each target, T cell engaging bispecifics hold T cells in close physical proximity with a cancer cell and allow for T cell proteins and enzymes to attack tumor cells and cause apoptosis, thereby killing cancer cells.

Epidermal growth factor receptor (EGFR), a receptor and transmembrane glycoprotein that exhibits intrinsic tyrosine kinase activity regulates numerous cellular processes including, but not limited to, activation of signal transduction pathways that control cell proliferation, differentiation, cell survival, apoptosis, angiogenesis, mitogenesis, and metastasis (Atalay et al., Ann. Oncology 14:1346-1363 (2003); Tsao and Herbst, Signal 4:4-9 (2003); Herbst and Shin, Cancer 94:1593-1611 (2002); Modjtahedi et al., Br. J. Cancer 73:228-235 (1996)). Overexpression of EGFR is associated with numerous human cancers, including cancers of the bladder, brain, head and neck, pancreas, lung, breast, ovary, colon, prostate, and kidney. EGFR is also expressed in the cells of normal tissues at lower levels than expressed in malignant cells.

Bispecific antibodies that engage EGFR and CD3 suffer from drawbacks, including T cell mediated toxicity (i.e., cytokine release) and EGFR-related toxicities due to off-tumor binding. Additionally, manufacturing challenges arise due to the complex structure of bispecific antibodies and high levels of aggregation during manufacturing and scale-up. Accordingly, there is a need for immunotherapeutic options which have an improved safety profile, as well as improved manufacturability.

### BRIEF SUMMARY

The present disclosure provides an activatable anti-EGFR, anti-CD3 heteromultimeric bispecific polypeptide complex (HBPC) comprising: (a) a first polypeptide comprising (i) a single-chain variable fragment (scFv) comprising a first heavy chain variable domain (VH1) and a first light chain variable domain (VL1), wherein the VH1 and the VL1 together form a T-cell cluster of differentiation (CD3)-targeting domain that specifically binds a CD3 polypeptide, (ii) a first masking moiety (MM1), (iii) a first cleavable moiety (CM1), (iv) a second heavy chain variable domain (VH2), and (v) a first monomeric Fc domain (Fc1); (b) a second polypeptide comprising (i) a second light chain variable domain (VL2), wherein the VH2 and the VL2 together form an EGFR targeting domain that specifically binds EGFR, (ii) a second masking moiety (MM2), and (iii) a second cleavable moiety (CM2); and (c) a third polypeptide that (i) comprises a second monomeric Fc domain (Fc2), and (ii) does not comprise an immunoglobulin variable domain. In some aspects, the CD3 polypeptide is the epsilon chain of CD3.

In some aspects of the activatable anti-EGFR, anti-CD3 heteromultimeric bispecific polypeptide disclosed herein, the VH1 comprises: (i) a VH CDR1 comprising the amino acid sequence KYAMN (SEQ ID NO:3), (ii) a VH CDR2 comprising the amino acid sequence RIRSKYNNYATYYADSVKD (SEQ ID NO:4), and (iii) a VH CDR3 comprising the amino acid sequence HGNFGNSYISYWAY (SEQ ID NO:5); and wherein the VL1 comprises: (i) a VL CDR1 comprising the amino acid sequence GSSTGAVTSGNYPN (SEQ ID NO:6), (ii) a VL CDR2 comprising the amino acid sequence GTKFLAP (SEQ ID NO:7), and (iii) a VL CDR3 comprising the amino acid sequence VLWYSNRWV (SEQ ID NO:8).

In some aspects, the scFv comprises a VH1 that has an amino acid sequence that is at least 90% identical to SEQ ID NO:9 and/or a VL1 that has an amino acid sequence that is at least 90% identical to SEQ ID NO:10. In some aspects, the scFv comprises a VH1 that has an amino acid sequence of SEQ ID NO:9 and a VL1 that has the amino acid sequence of SEQ ID NO:10.

In some aspects of the activatable anti-EGFR, anti-CD3 heteromultimeric bispecific polypeptide disclosed herein the VH2 comprises: (i) a VH CDR1 comprising the amino acid sequence NYGVH (SEQ ID NO:15), (ii) a VHCDR2 comprising the amino acid sequence VIWSGGNTDYNTPFTS (SEQ ID NO:16), and (iii) a VH CDR3 comprising the amino acid sequence ALTYYDYEFAY (SEQ ID NO:17). In some aspects, the VH2 comprises an amino acid sequence that is at least 90% identical to SEQ ID NO:21. In some aspects, the VH2 comprises an amino acid sequence of SEQ ID NO:21.

In some aspects of the activatable anti-EGFR, anti-CD3 heteromultimeric bispecific polypeptide disclosed herein, the Fc1 comprises an amino acid sequence that is at least 90% identical to SEQ ID NO:23. In some aspects, the Fc1 comprises the amino acid sequence of SEQ ID NO:23.

In some aspects of the activatable anti-EGFR, anti-CD3 heteromultimeric bispecific polypeptide disclosed herein, the first polypeptide further comprises a heavy chain CH1 domain between the VH2 and the Fc1. In some aspects, the first polypeptide further comprises an immunoglobulin hinge region between the VH2 and the Fc1. In some aspects, the first polypeptide comprises a structural arrangement from amino-terminus to carboxy-terminus of: MM1-CM1-scFv-VH2-CH1-hinge region-Fc1, wherein each “-” is independently a direct or indirect linkage.

In some aspects of the activatable anti-EGFR, anti-CD3 heteromultimeric bispecific polypeptide disclosed herein, the first polypeptide comprises one or more linkers. In some aspects, the linker comprises from about 1 to about 20 amino acids.

In some aspects of the activatable anti-EGFR, anti-CD3 heteromultimeric bispecific polypeptide disclosed herein, the VL2 comprises (i) a VL CDR1 comprising the amino acid sequence RASQSIGTNIH (SEQ ID NO:18), (ii) a VL CDR2 comprising the amino acid sequence YASESIS (SEQ ID NO:19), and (iii) a VL CDR3 comprising the amino acid sequence QQNNNWPTT (SEQ ID NO:20). In some aspects, the VL2 comprises an amino acid sequence that is at least 90% identical to SEQ ID NO:22. In some aspects, the VL2 comprises the amino acid sequence of SEQ ID NO:22.

In some aspects of the activatable anti-EGFR, anti-CD3 heteromultimeric bispecific polypeptide disclosed herein, the second polypeptide comprises a structural arrangement from amino-terminus to carboxy-terminus of: MM2-CM2-VL2, wherein each “-” is independently a direct or indirect linkage. In some aspects, the second polypeptide comprises one or more linkers. In some aspects, the linker comprises between about 1 and about 20 amino acids.

In some aspects of the activatable anti-EGFR, anti-CD3 heteromultimeric bispecific polypeptide disclosed herein, the Fc2 binds to the Fc1. In some aspects, the Fc2 comprises an amino acid sequence that is at least 90% identical to SEQ ID NO:28. In some aspects, the Fc2 comprises the amino acid sequence of SEQ ID NO:28. In some aspects, the Fc2 comprises the amino acid sequence of SEQ ID NO:29.

In some aspects of the activatable anti-EGFR, anti-CD3 heteromultimeric bispecific polypeptide disclosed herein, at least one of the first polypeptide and the third polypeptide further comprises an immunoglobulin hinge region. In some aspects, the first polypeptide and the third polypeptide comprises an immunoglobulin hinge region. In some aspects, the immunoglobulin hinge region of the first polypeptide and immunoglobulin hinge region of the third polypeptide comprises the same amino acid sequence. In some aspects, the immunoglobulin hinge region of the first polypeptide and immunoglobulin hinge region of the third polypeptide comprise different amino acid sequences.

In some aspects of the activatable anti-EGFR, anti-CD3 heteromultimeric bispecific polypeptide disclosed herein, the third polypeptide comprises an immunoglobulin hinge region in a structural arrangement from amino-terminus to carboxy-terminus of: hinge region-Fc2.

In some aspects of the activatable anti-EGFR, anti-CD3 heteromultimeric bispecific polypeptide disclosed herein, the first, second, and/or third polypeptide comprise one or more linkers. In some aspects, MM1 is linked to CM1 via a linker L1. In some aspects, MM2 is linked to CM2 via a linker L2. In some aspects, the amino acid sequence of L1 and L2 are the same. In some aspects, the amino acid sequence of L1 and L2 are different.

In some aspects of the activatable anti-EGFR, anti-CD3 heteromultimeric bispecific polypeptide disclosed herein, the CM1 and the CM2 each comprise a substrate for a protease that is present in a tumor microenvironment of a subject having cancer. In some aspects, the CM1 and the CM2 each comprise a substrate for the same protease. In some aspects, the CM1 and the CM2 comprise substrates for different proteases. In some aspects, CM1 and CM2 each independently comprise a substrate for a protease selected from the group of proteases shown in Table 2. In some aspects, at least one of the CM1 and CM2 comprises a substrate for a serine protease or matrix metallopeptidase (MMP). In some aspects, CM1 comprises the amino acid sequence SEQ ID NO:2 and/or CM2 comprises the amino acid sequence SEQ ID NO:14. In some aspects, CM1 comprises the amino acid sequence of SEQ ID NO:2. In some aspects, CM2 comprises the amino acid sequence of SEQ ID NO:14.

In some aspects of the activatable anti-EGFR, anti-CD3 heteromultimeric bispecific polypeptide disclosed herein, the MM1 and/or the MM2 comprises between about 5 amino acids to about 40 amino acids. In some aspects, the MM1 is selected from the group consisting of: SEQ ID NO:1, SEQ ID NO:67, SEQ ID NO:68, SEQ ID NO:69, SEQ ID NO:70, SEQ ID NO:71, or SEQ ID NO:72. In some aspects, MM2 comprises the amino acid sequences of SEQ ID NO:13. In some aspects, wherein MM1 comprises the amino acid sequence of SEQ ID NO:1.

In some aspects of the activatable anti-EGFR, anti-CD3 heteromultimeric bispecific polypeptide disclosed herein, at least one of the one or more linkers is selected from the group consisting of: (i) a glycine-serine-based linker selected from the group consisting of (GS)n, wherein n is an integer of at least 1, (GGS)n, wherein n is an integer of at least 1 (e.g., an integer from about 1 to about 20, or from about 1 to about 10), (GGGS)n (SEQ ID NO:40), wherein n is an integer of at least 1 (e.g., an integer from about 1 to about 20, or from about 1 to about 10), (GGGGS)n (SEQ ID NO:126), where n is an integer of at least 1 (e.g., an integer from about 1 to about 20, or from about 1 to about 10), (GSGGS)n (SEQ ID NO:41), wherein n is an integer of at least 1 (e.g., an integer from about 1 to about 20, or from about 1 to about 10), GSSGGSGGSG (SEQ ID NO:12), GGSG (SEQ ID NO:42), GGSGG (SEQ ID NO:43), GSGSG (SEQ ID NO:44), GSGGG (SEQ ID NO:45), GGGSG (SEQ ID NO:46), and GSSSG (SEQ ID NO:47), GGGGSGGGGSGGGGSGS (SEQ ID NO:48), GGGGSGS (SEQ ID NO:49), GGGGSGGGGSGGGGS (SEQ ID NO:50), GGGGSGGGGSGGGGSGGGGS (SEQ ID NO:51), GGGGS (SEQ ID NO:52), GGGGSGGGGS (SEQ ID NO:53), GGGS (SEQ ID NO:54), GGGSGGGS (SEQ ID NO:55), GGGSGGGSGGGS (SEQ ID NO:56), GSSGGSGGSGG (SEQ ID NO:57), GGGSGGGGSGGGGSGGGGSGGGGS (SEQ ID NO:58), GGGSSGGS (SEQ ID NO:127) and GS; and (ii) a linker comprising glycine and serine, and at least one of lysine, threonine, or proline selected from the group consisting of GSTSGSGKPGSSEGST (SEQ ID NO:59), SKYGPPCPPCPAPEFLG (SEQ ID NO:60), GGSLDPKGGGGS (SEQ ID NO:61), PKSCDKTHTCPPCPAPELLG (SEQ ID NO:62), GKSSGSGSESKS (SEQ ID NO:63), GSTSGSGKSSEGKG (SEQ ID NO:64), GSTSGSGKSSEGSGSTKG (SEQ ID NO:65), and GSTSGSGKPGSGEGSTKG (SEQ ID NO:66).

In some aspects of the activatable anti-EGFR, anti-CD3 heteromultimeric bispecific polypeptide disclosed herein, (1) the first polypeptide comprises the amino acid sequence of SEQ ID NO:30, (2) the second polypeptide comprises the amino acid sequence of SEQ ID NO:31, and (3) the third polypeptide comprises the amino acid sequence of SEQ ID NO:32.

In some aspects of the activatable anti-EGFR, anti-CD3 heteromultimeric bispecific polypeptide disclosed herein, (1) the first polypeptide comprises the amino acid sequence of SEQ ID NO:120, (2) the second polypeptide comprises the amino acid sequence of SEQ ID NO:37, and (3) the third polypeptide comprises the amino acid sequence of SEQ ID NO:32.

Disclosed herein is a pharmaceutical composition comprising the activatable bispecific polypeptide complex of disclosed herein and a pharmaceutically acceptable carrier.

Also disclosed herein is a kit comprising the pharmaceutical composition comprising the activatable bispecific polypeptide complex of disclosed herein and a pharmaceutically acceptable carrier.

Also disclosed herein is a nucleic acid comprising nucleotide sequences that encode the first polypeptide, the second polypeptide, and the third polypeptide of the activatable bispecific polypeptide described herein. Further provided herein are vectors comprising the nucleic acid described and host cells comprising the vectors.

Also disclosed herein is a method of producing an activatable bispecific polypeptide complex comprising: (a) culturing a host cell in a liquid culture medium under conditions sufficient to produce the activatable bispecific polypeptide complex; and (b) recovering the activatable bispecific polypeptide complex.

Also disclosed herein is a method of treating a disease in a subject comprising administering a therapeutically effective amount of the activatable bispecific polypeptide complex described herein or the pharmaceutical composition described herein to the subject. In some aspects, the subject is a human. In some aspects, the disease is a cancer. In some aspects, the activatable bispecific polypeptide or the pharmaceutical composition is for use in inhibiting tumor growth in a subject in need thereof.

Also disclosed herein is the use of an activatable bispecific polypeptide complex described herein, or the pharmaceutical composition described herein, in the manufacture of a medicament for treating cancer.

### BRIEF DESCRIPTION OF THE FIGURES

FIG. 1 is a schematic of an activatable anti-EGFR, anti-CD3 heteromultimeric bispecific polypeptide complex (HBPC) described herein.

FIG. 2A shows binding to EGFR by CI106 (an activatable double-arm, divalent anti-CD3, anti-EGFR bispecific antibody control), Complex-57 (an activatable HBPC) and Complex-67 (an activatable HBPC), as well as activated CI106, activated Complex-57, and activated Complex-67.

FIG. 2B shows binding to CD3 by CI106 (control), Complex-57 (an activatable HBPC), Complex-67 (an activatable HBPC) and activated CI106, activated Complex-57, and activated Complex-67.

FIG. 3A shows cytotoxicity to HT29 cells following treatment with activated CI106 (control), Complex-57, and Complex-67, and CI106 (double-arm, divalent bispecific control construct) and Complex-57.

FIG. 3B shows cytotoxicity to HT29 cells following treatment with CI106 (control), Complex-67, activated CI106 (control) and activated Complex-67.

FIG. 4 shows tumor volume in a HT29-luc2 xenograft tumor model as a function of time following treatment with Vehicle, 1.0 mg/kg CI106 (control) and 0.2, 0.6, and 1.8 mg/kg Complex-67.

FIG. 5 shows tumor volume in a HCT116 xenograft tumor model as a function of time following treatment with Vehicle, 0.3 mg/kg and 1 mg/kg activated Complex-67 and Complex-67.

FIG. 6 shows percentage (%) monomer versus concentration for CI106 (control), Complex-57 and Complex-67.

FIGS. 7A-7C show the flow cytometry assessment of CI107 binding to EGFR and CD3 expressed on the surface of HT29 cells (A), HCT116 cells (B), or Jurkat cells (C). The apparent Kd was calculated from duplicate experiments in HT29 cells and triplicate experiments in Jurkat cells.

FIGS. 8A-8D show the percent cytotoxicity mediated by CI107 in HCT116-Luc2 cells (A, C) and HT29-Luc2 cells (B, D). After 48 hours of culture, HCT116-Luc2 or HT29-Luc2 cell viability and cytotoxicity were measured relative to untreated controls (A, B). After 16 hours of culture, CD69 expression was measured by flow cytometry. MFI, mean fluorescence intensity (C, D).

FIGS. 9A-9E show cytokine release following treatment with CI107, measured after 16 hours of culture. (A) IFN-γ, (B) IL-2, (C) IL-6, (D) MCP-1, and (E) TNF-α.

FIGS. 10A-10B shows the tumor volume after treatment with test TCBs in mice harboring HT29-Luc2 tumors and engrafted with human PBMCs. (A) Mice were treated once weekly for 3 weeks with vehicle (PBS) or 0.3 mg/kg CI020, CI011, CI040, or CI048 (n=8 per group). Tumor volume was measured twice weekly. (B) NSG mice harboring HT29-Luc2 tumors and engrafted with human PBMCs were treated with vehicle or 1 mg/kg of CI020, CI011, CI040, or CI048. Tumors were harvested 7 days after dosing, and immunohistochemistry for CD3 was performed. Dark staining indicates CD3+ cells.

FIGS. 11A-11B show tumor volumes following treatment with CI107 once weekly for 3 weeks in HT29 (A) and HCT116 (B) xenograft tumors. Tumor volume was measured twice weekly. * p<0.5; ** p<0.01; **** p<0.0001.

FIGS. 12A-12B show levels of IL-6 (A) and IFN-γ (B) measured 8 hours after dosing with CI107.

FIG. 12C shows levels of aspartate aminotransferase (AST) measured by serum chemistry analysis 48 hours after dosing with CI107 (C).

FIG. 12D shows plasma concentrations of Act-CI107 and CI107 measured by ELISA using anti-idiotype capture and anti-human Fc detection. CI107 lines represent data from 3 individual animals dosed with 2.0 mg/kg CI107; Act-TCB lines represent single animals dosed with 0.06 mg/kg or 0.18 mg/kg Act-TCB.

### DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.

### Definitions

As used herein, the term “heteromultimeric bispecific polypeptide complex” and “HBPC” are used interchangeably to refer to a set of polypeptides that together form a complex that has binding domains that are capable of binding to two different biological targets.

The term “activatable” when used in connection with the term “heteromultimeric bispecific polypeptide complex” or “HBPC” refers herein to an HBPC whose binding activity is impaired by the presence of masking moieties appended to the structure of the HBPC. The terms “activated” and “act-” can each be used to refer to an activated HBPC. The terms “activated” and “unmasked,” are used interchangeably herein.

As used herein, the term “EGFR” refers to a receptor and transmembrane glycoprotein and a member of the protein kinase superfamily. Human epidermal growth factor receptor is a 170 kDa transmembrane receptor encoded by the c-erb B-1 protooncogene, and exhibits intrinsic tyrosine kinase activity (Modjtahedi et al., Br. J. Cancer 73:228-235 (1996); Herbst and Shin, Cancer 94:1593-1611 (2002)). There are also known isoforms and variants of EGFR (e.g., alternative RNA transcripts, truncated versions, polymorphisms, etc.), which are contemplated for use herein. EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay et al., Ann. Oncology 14:1346-1363 (2003); Tsao and Herbst, Signal 4:4-9 (2003); Herbst and Shin, Cancer 94:1593-1611 (2002); Modjtahedi et al., Br. J. Cancer 73:228-235 (1996)). Overexpression of EGFR is associated with numerous human cancers, including cancers of the bladder, brain, head and neck, pancreas, lung, breast, ovary, colon, prostate, and kidney. EGFR is also expressed in the cells of normal tissues at lower levels than expressed in malignant cells. Exemplary anti-EGFR antigen-binding proteins include but are not limited to human wildtype EGFR (NCBI Accession No. NG_007726.E), human wildtype EGFR Transcript Variant 1 (NCBI Accession No. NP_005219.2), human wildtype EGFR Transcript Variant 2 (NCBI Accession No. NP_958439.1), human wildtype EGFR Transcript Variant 3 (NCBI Accession No. NP_958440.1), human wildtype EGFR Transcript Variant 4 (NCBI Accession No. NP_958441.1), human wildtype EGFR Transcript Variant 5 (NCBI Accession No. NP_001333826.1), human wildtype EGFR Transcript Variant 6 (NCBI Accession No. NP_001333827.1), human wildtype EGFR Transcript Variant 7 (NCBI Accession No. NP_001333828.1), human wildtype EGFR Transcript Variant 8 (NCBI Accession No. NM_001346941.2), human wildtype EGFR Transcript Variant EGFRvIII (NCBI Accession No. NP_001333870.1), and the like.

The term “CD3” or “cluster of differentiation 3” as used herein refers to a protein complex of six chains which are subunits of the T cell receptor complex. (Janeway et al., p. 166, 9^{th} ed.) The TCR α:β heterodimer associates with CD3 subunits to complete the TCR cell-surface antigen receptor. Two CD3ε chains, a CD3γ chain, and a CD3δ chain and a homodimer of CD3ζ chains complete the T cell receptor complex, which is involved in the recognition of peptides bound to the major histocompatibility complex class I and II and involves T cell activation. The CD3 antigen is expressed by mature T lymphocytes and by a subset of thymocytes. The CD3-targeting domain that specifically binds a CD3 polypeptide, disclosed herein, can be from any vertebrate source, including mammals such as primates (e.g. humans) and rodents (e.g., mice and rats). The term encompasses “full-length,” unprocessed CD3 (e.g., unprocessed or unmodified CD3ε or CD3γ) as well as any form of CD3 that results from processing in the cell. The term also encompasses naturally occurring variants of CD3, including, for example, splice variants or allelic variants. An anti-CD3 targeting domain described herein can specifically bind to human wildtype CD3E (NCBI Accession No. NM_000733.3).

The term “T cell,” as used herein is defined as a thymus-derived lymphocyte that participates in a variety of cell-mediated immune reactions. The term “regulatory T cell” as used herein refers to a CD4⁺CD25⁺FoxP3⁺ T cell. “Treg” is the abbreviation used herein for a regulatory T cell.

The term “helper T cell” as used herein refers to a CD4⁺ T cell; helper T cells recognize antigen bound to MHC Class II molecules. There are at least two types of helper T cells, Th1 and Th2, which produce different cytokines. Helper T cells become CD25⁺ when activated, but only transiently become FoxP3⁺.

The term “cytotoxic T cell” as used herein refers to a CD8⁺ T cell; cytotoxic T cells recognize antigen bound to MHC Class I molecules.

The term “variable region” or “variable domain” refers to the domain of an antigen binding protein (e.g., an antibody) heavy or light chain that is involved in binding the antigen binding protein (e.g., antibody) to antigen. The variable regions or domains of the heavy chain and light chain (VH and VL, respectively) of an antigen binding protein such as an antibody can be further subdivided into regions of hypervariability (or hypervariable regions, which may be hypervariable in sequence and/or form of structurally defined loops), such as hypervariable regions (HVRs) or complementarity-determining regions (CDRs), interspersed with regions that are more conserved, termed framework regions (FRs). In general, there are three HVRs (HVR-H1, HVR-H2, HVR-H3) or CDRs (CDR-H1, CDR-H2, CDR-H3) in each heavy chain variable region, and three HVRs (HVR-L1, HVR-L2, HVR-L3) or CDRs in (CDR-L1, CDR-L2, CDR-L3) in each light chain variable region. “Framework regions” and “FR” are known in the art to refer to the non-HVR or non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each VH and VL, three HVRs or CDRs and four FRs are typically arranged from amino-terminus to carboxy-terminus in the following order: FR1, HVR1, FR2, HVR2, FR3, HVR3, FR4 in the case of HVRs, or FR1, CDR1, FR2, CDR2, FR3, CDR3, FR4 in the case of CDRs (See also Chothia and Lesk J. Mot. Biol., 195, 901-917 (1987)). A single VH or VL domain can be sufficient to confer antigen-binding specificity. In addition, antibodies that bind a particular antigen can be isolated using a VH or VL domain from an antibody that binds the antigen to screen a library of complementary VL or VH domains, respectively. See, e.g., Portolano et al. J. Immunol. 150:880-887 (1993); Clarkson et al., Nature 352:624-628 (1991).

The term “heavy chain variable region” (VH) as used herein refers to a region comprising heavy chain HVR-H1, FR-H2, HVR-H2, FR-H3, and HVR-H3. For example, a heavy chain variable region may comprise heavy chain CDR-H1, FR-H2, CDR-H2, FR-H3, and CDR-H3. In some aspects, a heavy chain variable region also comprises at least a portion of an FR-H1 and/or at least a portion of an FR-H4.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ.

The term “light chain variable region” (VL) as used herein refers to a region comprising light chain HVR-L1, FR-L2, HVR-L2, FR-L3, and HVR-L3. In some aspects, the light chain variable region comprises light chain CDR-L1, FR-L2, CDR-L2, FR-L3, and CDR-L3. In some aspects, a light chain variable region also comprises an FR-L1 and/or an FR-L4.

The term “light chain constant region” as used herein refers to a region comprising a light chain constant domain, C_{L}. Nonlimiting exemplary light chain constant regions include λ and κ.

The term “light chain” (LC) as used herein refers to a polypeptide comprising at least a light chain variable region, with or without a leader sequence. In some aspects, a light chain comprises at least a portion of a light chain constant region. The term “full-length light chain” as used herein refers to a polypeptide comprising a light chain variable region and a light chain constant region, with or without a leader sequence.

The term “antibody” refers to an immunoglobulin molecule or an immunologically active portion of an immunoglobulin (Ig) molecule, i.e., a molecule that contains an antigen binding site that specifically binds (immunoreacts with) an antigen. An “antigen-binding portion” of an antibody or polypeptide (also called an “antigen-binding fragment”) refers to one or more portions of an antibody or polypeptide that bind specifically to the target antigen. Antibodies and antigen-binding portions include, but are not limited to, polyclonal, monoclonal, chimeric, domain antibody, single chain antibodies, Fab, and F(ab′)₂ fragments, scFvs, Fd fragments, Fv fragments, single domain antibody (sdAb) fragments, dual-affinity re-targeting antibodies (DARTs), dual variable domain immunoglobulins; isolated complementarity determining regions (CDRs), and a combination of two or more isolated CDRs, which can optionally be joined by a synthetic linker, and a Fab expression library. A nonhuman antibody, e.g., a camelid antibody, may be humanized by recombinant methods to reduce its immunogenicity in a human.

The CDR sequences specified herein are determined in accordance with the Kabat numbering system (i.e., the “Kabat CDRs”) as described in Abhinandan, K. R. and Martin, A. C. R. (2008) “Analysis and improvements to Kabat and structurally correct numbering of antibody variable domains”, Molecular Immunology, 45, 3832-3839, which is incorporated herein by reference in its entirety. The Kabat CDRs are defined as CDR-L1: residues L24-L34; CDR-L2: residues L50-L56; CDR-L3: residues L89-L97; CDR-H1: residues H31-H35; CDR-H2: residues H50-H65; and CDR-H3: residues H95-H102, where “L” refers to the light chain variable domain and “H” refers to the heavy chain variable domain.

“Specifically binds” or “immunospecifically binds” means that the targeting domain, antibody or antigen-binding fragment reacts with one or more antigenic determinants of the desired antigen and does not react with other polypeptides or binds at much lower affinity (Kd >10⁻⁶), wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (kₒₙ) and the “off rate constant” (k_{off}) can be determined by calculation of the concentrations and the actual rates of association and dissociation. (See Nature 361:186-87 (1993)). The ratio of k_{off}/kₒₙ enables the cancellation of all parameters not related to affinity, and is equal to the dissociation constant Kd. (See, generally, Davies et al. (1990) Annual Rev Biochem 59:439-473). In some aspects, the antigen-targeting domain, antibody, or antigen-binding fragment that specifically binds to its corresponding antigen exhibits a Kd of less than about 10 μM, and in some aspects, less than about 100 μM with respect to the target antigen.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

An “anti-antigen” antibody or polypeptide refers to an antibody or polypeptide that binds specifically to the antigen. For example, an anti-CD3 polypeptide binds specifically to CD3.

As used herein, the terms “MM” and “masking moiety” are used interchangeably to refer to a peptide that interferes with binding of the targeting domain to its corresponding antigen. For example, MM1 is a peptide that interferes with binding of the first targeting domain to the first target and MM2 is a peptide that interferes with binding of the second targeting domain to the second target. The extent to which a masking moiety interferes with the binding of the targeting domain to its corresponding target is quantified by its “masking efficiency.” The terms “masking efficiency” and “ME” are used interchangeably herein to refer to a ratio that is determined as follows:

$$ME = \frac{EC 50 , activatable HBPC ( i . e . , not cleaved by protease )}{EC 50 , activated HBPC}$$

As used herein, the terms “CM” and “cleavable moiety” are used interchangeably to refer to a peptide substrate that is susceptible to cleavage by a protease that is upregulated in tumor cells. Protease-mediated cleavage of the CM results in the release of the MM from the structure of the activatable HBPC, thereby generating an “activated” (i.e., unmasked) product, where each corresponding “activated” (i.e, unmasked) first and/or second targeting domain is free to bind its respective target.

The term “isolated polynucleotide” as used herein refers to a recombinant polynucleotide or polynucleotide of synthetic origin which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the disclosure include the nucleic acid molecules encoding the first, second, and third polypeptides.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

As discussed herein, minor variations in the amino acid sequences described herein (i.e., each reference sequence) are contemplated as being encompassed by the present disclosure, provided that the resulting analog sequence maintains at least 75%, more preferably at least 80%, 90%, 95%, and most preferably 99% sequence identity to the reference sequence. In particular, conservative amino acid replacements are contemplated. Conservative replacements are those that take place within a family of amino acids that are related with respect to the nature of their side chains. Amino acids may be divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, within the HBPC polypeptides and polypeptide complexes described herein, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a CDR or framework region. Whether an amino acid change results in a functional polypeptide complex can readily be determined by assaying the specific activity of the resulting molecule, i.e., the resulting analog sequence. Assays are described in detail herein. Preferred amino- and carboxy-termini of analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

A conservative amino acid substitution should not substantially change the structural characteristics of the reference sequence (e.g., a replacement amino acid should not tend to break a helix that occurs in the reference sequence, or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

Exemplary amino acid substitutions also include those which: (1) reduce susceptibility to proteolysis in regions of the activatable anti-EGFR, anti-CD3 heteromultimeric bispecific polypeptide other than in the cleavable linker comprising the CM, (2) reduce susceptibility to oxidation, (3) alter binding affinity for forming protein complexes, (4) alter binding affinities to antigen, and (4) confer or modify other physicochemical or functional properties of such analogs. Such amino acid substitutions may be identified using known mutagenesis methods and/or directed molecular evolution methods using the assays described herein. See, e.g., International Publication No. WO 2001/032712, U.S. Pat. No. 7,432,083, U.S. Pub. No. 2004/0180340, and U.S. Pat. No. 6,297,053, each of which is incorporated herein by reference. Analogs may be prepared by introducing one or more mutations in a reference sequence within an activatable HBPC. For example, single or multiple amino acid substitutions may be made in the reference sequence (preferably in the portion of the polypeptide outside the domain(s) forming intermolecular contacts).

As used herein, by “pharmaceutically acceptable” or “pharmacologically compatible” is meant a material that is not biologically or otherwise undesirable, e.g., the material may be incorporated into a pharmaceutical composition administered to an individual or subject without causing any significant undesirable biological effects or interacting in a deleterious manner with any of the other components of the composition in which it is contained. Pharmaceutically acceptable carriers or excipients have for example met the required standards of toxicological and manufacturing testing and/or are included on the Inactive Ingredient Guide prepared by the U.S. Food and Drug administration.

A “patient” as used herein includes any patient who is afflicted with a cancer. The terms “subject” and “patient” are used interchangeably herein.

The terms “cancer,” “cancerous,” or “malignant” refer to or describe the physiological condition in mammals that is typically characterized by unregulated cell growth. Examples of cancer include, for example, melanoma, such as unresectable or metastatic melanoma, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

The term “tumor” as used herein refers to any mass of tissue that results from excessive cell growth or proliferation, either benign (non-cancerous) or malignant (cancerous), including pre-cancerous lesions.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, in some aspects, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of a tumor. Effective treatment may refer to alleviation of at least one symptom associated with a cancer. Such effective treatment may, e.g., reduce patient pain, reduce the size and/or number of lesions, may reduce or prevent metastasis of a tumor, and/or may slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some aspects, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit, slow to some extent and may stop tumor metastasis; (v) inhibit tumor growth; (vi) prevent or delay occurrence and/or recurrence of tumor; and/or (vii) relieve to some extent one or more of the symptoms associated with the cancer.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver, spleen, and/or bone marrow (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the above-defined terms are more fully defined by reference to the specification in its entirety.

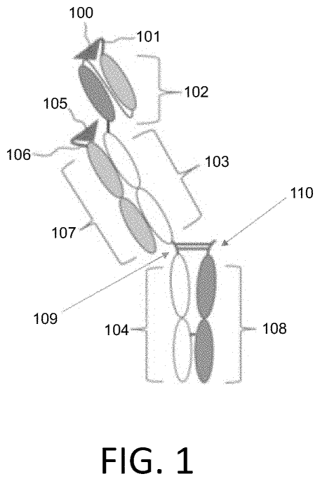

Schematic representations of activatable polypeptides of the present disclosure, e.g., FIG. 1, are not intended to be exclusive. Other sequence elements, such as linkers, spacers, and signal sequences, may be present before, after, or between the listed sequence elements in such schematic representations. It is also to be appreciated that a MM and a CM can be joined to a VH of an antibody or polypeptide instead of to a VL of an antibody or polypeptide, and vice versa.

Various aspects of the disclosure are described in further detail in the following subsections.

### Activatable Anti-EGFR, Anti-CD3 Heteromultimeric Bispecific Polypeptide Complex

The present disclosure provides an activatable anti-EGFR, anti-CD3 heteromultimeric bispecific polypeptide (HBPC) comprising: (a) a first polypeptide comprising (i) a single-chain variable fragment (scFv) comprising a first heavy chain variable domain (VH1) and a first light chain variable domain (VL1), wherein the VH1 and the VL1 together form a T-cell cluster of differentiation (CD3)-targeting domain that specifically binds a CD3 polypeptide, (ii) a first masking moiety (MM1), (iii) a first cleavable moiety (CM1), (iv) a second heavy chain variable domain (VH2), and (v) a first monomeric Fc domain (Fc1); (b) a second polypeptide comprising (i) a second light chain variable domain (VL2), wherein the VH2 and the VL2 together form an EGFR targeting domain that specifically binds EGFR, (ii) a second masking moiety (MM2), and (iii) a second cleavable moiety (CM2); and (c) a third polypeptide that (i) comprises a second monomeric Fc domain (Fc2), and (ii) does not comprise an immunoglobulin variable domain, and wherein the MM1 is a peptide that interferes with binding of the CD3-targeting domain to a CD3 polypeptide and MM2 is a peptide that interferes with binding of the EGFR-targeting domain to an EGFR. As demonstrated in the Examples herein, activatable HBPCs of the present disclosure provide advantages over activatable or masked molecules known in the art, including aggregation resistance, low levels of concentration dependent aggregation, which is particularly beneficial during purification where relatively high concentrations of activatable HBPC product may be generated, high potency when activated, and improved anti-tumor activity (when activated).

As described herein above, among the components present in the first polypeptide of the activatable anti-EGFR, anti-CD3 HBPC is a T-cell CD3-targeting domain comprising a single-chain variable fragment (scFv) that specifically binds a CD3 polypeptide. In some aspects, the CD3 polypeptide is the epsilon chain of CD3. In some aspects, the scFv (anti-CD3 scFv) employed herein comprises a heavy chain variable domain (VH1) and a light chain variable domain (VL1).

The VH1 comprises a variable heavy chain CDR1 (VH CDR1, also referred to herein as CDRH1), a variable heavy chain CDR2 (VH CDR2, also referred to herein as CDRH2), and a variable heavy chain CDR3 (VH CDR3, also referred to herein as CDRH3), the VL1 comprises a variable light chain CDR1 (VL CDR1, also referred to herein as CDRL1), a variable light chain CDR2 (VL CDR2, also referred to herein as CDRL2), and a variable light chain CDR3 (VL CDR3, also referred to herein as CDRL3).

The activatable anti-EGFR, anti-CD3 HBPC provided herein comprises a masking moiety (MM). As used herein, the terms “masking moiety” and “MM”, are used interchangeably to refer to a peptide that, when positioned proximal to a targeting domain, interferes with binding of the targeting domain to its target. In some aspects, the MM is an amino acid sequence that is coupled, or otherwise attached, to the activatable anti-EGFR, anti-CD3 HBPC and is attached to the activatable anti-EGFR, anti-CD3 HBPC such that each MM reduces the ability of the activatable anti-EGFR, anti-CD3 HBPC to specifically bind to its targets. In some aspects, MM1 prevents or decreases the ability of the activatable anti-EGFR, anti-CD3 HBPC from specifically binding to CD3. In some aspects, MM2 prevents or decreases the ability of the activatable anti-EGFR, anti-CD3 HBPC from specifically binding to EGFR. In some aspects, the MM binds specifically to the antigen targeting domain(s). Suitable MMs can be identified using any of a variety of known techniques.

For example, anti-EGFR masking moieties that are suitable for use in the practice of the present disclosure in connection with a variety of antibody binding domains include any that are known in the art, including those described in, for example, PCT Publication Nos. WO 2013/163631, WO 2015/013671, WO 2016/014974, WO 2019/075405, and WO 2019/213444, each of which are incorporated herein by reference in their entireties. Anti-CD3 masking moieties that are suitable for use in the practice of the present disclosure include any of those that are known in the art, including those described in, for example, WO2013/163631, WO 2015/013671, WO 2016/014974, WO 2019/075405, and WO 2019/213444, each of which is incorporated herein by reference in their entireties.

In some aspects of the activatable anti-EGFR, anti-CD3 HBPC provided herein, the MM1 and/or the MM2 comprises from 5 amino acids to about 40 amino acids, or any range therebetween, and including both 5 amino acids and 40 amino acids. As used herein, the term “MM1” indicates a masking moiety for the CD3 targeting domain. As used herein, the term “MM2” indicates a masking moiety on the EGFR targeting domain.

In some aspects of the activatable anti-EGFR, anti-CD3 HBPC provided herein, MMI is selected from the group consisting of SEQ ID NOs: 1, 67, 68, 69, 70, 71, and 72. In some aspects, MM1 comprises the amino acid sequence of SEQ ID NO:1. In some aspects, MM2 comprises the amino acid sequence of SEQ ID NO:13. In some aspects, MM1 comprises SEQ ID NO:1 and MM2 is SEQ ID NO:13. In some aspects, MM1 comprises SEQ ID NO:72 and MM2 comprises SEQ ID NO:13.

In some aspects of the present disclosure, the single-chain variable fragment comprise a heavy chain variable domain (VH1) comprising: (i) a VH CDR1 comprising the amino acid sequence KYAMN (SEQ ID NO:3), (ii) a VH CDR2 comprising the amino acid sequence RIRSKYNNYATYYADSVKD (SEQ ID NO:4), and (iii) a VH CDR3 comprising the amino acid sequence HGNFGNSYISYWAY (SEQ ID NO:5); and a light chain variable domain (VL1) comprising (i) a VL CDR1 comprising the amino acid sequence GSSTGAVTSGNYPN (SEQ ID NO:6), (ii) a VL CDR2 comprising the amino acid sequence GTKFLAP (SEQ ID NO:7), and (iii) a VL CDR3 comprising the amino acid sequence VLWYSNRWV (SEQ ID NO:8).

In some aspects of the present disclosure, a VH1 comprises the amino acid sequence of SEQ ID NO:9. In some aspects of the present disclosure, a VL1 comprises the amino acid sequence of SEQ ID NO:10. In a specific aspect of the present disclosure, the scFv comprises the amino acid sequence of SEQ ID NO:11 (which comprises SEQ ID NOs: 9 and 10).

In some aspects of the present disclosure, VH1 comprises an amino acid sequence that is at least 90% identical, at least 91%, at least 92%, at least 93%, at least 94%, at least 95%, at least 96%, at least 97%, at least 98%, or at least 99% identical to SEQ ID NO:9. In some aspects of the present disclosure, VL1 comprises an amino acid sequence that is at least 90% identical, at least 91%, at least 92%, at least 93%, at least 94%, at least 95%, at least 96%, at least 97%, at least 98%, or at least 99% identical to SEQ ID NO:10.

In some aspects of the present disclosure, the first polypeptide single-chain variable fragment comprises a heavy chain variable domain (VH1) comprising: (i) a VH CDR1 comprising the amino acid sequence KYAMN (SEQ ID NO:3), (ii) a VH CDR2 comprising the amino acid sequence RIRSKYNNYATYYADSVKD (SEQ ID NO:4), (iii) a VH CDR3 comprising the amino acid sequence HGNFGNSYISYWAY (SEQ ID NO:5), and comprises a heavy chain variable domain at least 90% identical, at least 91%, at least 92%, at least 93%, at least 94%, at least 95%, at least 96%, at least 97%, at least 98%, or at least 99% identical to SEQ ID NO:9.

In some aspects of the present disclosure, VL1 comprises an amino acid sequence that comprises (i) a VL CDR1 comprising the amino acid sequence GSSTGAVTSGNYPN (SEQ ID NO:6), (ii) a VL CDR2 comprising the amino acid sequence GTKFLAP (SEQ ID NO:7), (iii) a VL CDR3 comprising the amino acid sequence VLWYSNRWV (SEQ ID NO:8), wherein the amino acid sequence of VL1 is at least 90% identical, at least 91%, at least 92%, at least 93%, at least 94%, at least 95%, at least 96%, at least 97%, at least 98%, or at least 99% identical to SEQ ID NO:10.

In some aspects, when the VH1 comprises: (i) a VH CDR1 comprising the amino acid sequence KYAMN (SEQ ID NO:3), (ii) a VH CDR2 comprising the amino acid sequence RIRSKYNNYATYYADSVKD (SEQ ID NO:4), and (iii) a VH CDR3 comprising the amino acid sequence HGNFGNSYISYWAY (SEQ ID NO:5); and the VL1 comprises (i) a VL CDR1 comprising the amino acid sequence GSSTGAVTSGNYPN (SEQ ID NO:6), (ii) a VL CDR2 comprising the amino acid sequence GTKFLAP (SEQ ID NO:7), and (iii) a VL CDR3 comprising the amino acid sequence VLWYSNRWV (SEQ ID NO:8), the MM1 comprises the amino acid sequence of SEQ ID NO:1.

In an alternative aspect, the single-chain variable fragment comprises a heavy chain variable domain (VH1) comprising: (i) a VH CDR1 comprising the amino acid sequence TYAMN (SEQ ID NO:128), (ii) a VH CDR2 comprising the amino acid sequence RIRSKYNNYATYYADSVKD (SEQ ID NO:129) and (iii) a VH CDR3 comprising the amino acid sequence HGNFGNSYVSWFAY (SEQ ID NO:130); and a light chain variable domain (VL1) comprising (i) a VL CDR1 comprising the amino acid sequence RSSTGAVTTSNYAN (SEQ ID NO:131), (ii) a VL CDR2 comprising the amino acid sequence GTNKRAP (SEQ ID NO:132) (iii) a VL CDR3 comprising the amino acid sequence ALWYSNLWV (SEQ ID NO:133).

In some of these aspects of the present disclosure, VH1 comprises the amino acid sequence of SEQ ID NO:134. In certain aspects of the present disclosure, VL1 comprises the amino acid sequence of SEQ ID NO:135. In a specific aspect of the present disclosure, the scFv comprises the amino acid sequence of SEQ ID NO:122 (which comprises SEQ ID NOs: 134 and 135).

In some aspects of the present disclosure, VH1 comprises an amino acid sequence that is at least 90% identical, at least 91%, at least 92%, at least 93%, at least 94%, at least 95%, at least 96%, at least 97%, at least 98%, or at least 99% identical to SEQ ID NO:134. In some aspects of the present disclosure, VL1 comprises an amino acid sequence that is at least 90% identical, at least 91%, at least 92%, at least 93%, at least 94%, at least 95%, at least 96%, at least 97%, at least 98%, or at least 99% identical to SEQ ID NO:135.

In some aspects of the present disclosure, the first polypeptide single-chain variable fragment comprises a heavy chain variable domain (VH1) comprising: (i) a VH CDR1 comprising the amino acid sequence TYAMN (SEQ ID NO:128), (ii) a VH CDR2 comprising the amino acid sequence RIRSKYNNYATYYADSVKD (SEQ ID NO:129), (iii) a VH CDR3 comprising the amino acid sequence HGNFGNSYVSWFAY (SEQ ID NO:130), and comprises a heavy chain variable domain at least 90% identical, at least 91%, at least 92%, at least 93%, at least 94%, at least 95%, at least 96%, at least 97%, at least 98%, or at least 99% identical to SEQ ID NO:135.

In some aspects of the present disclosure, VL1 comprises an amino acid sequence that comprises (i) a VL CDR1 comprising the amino acid sequence RSSTGAVTTSNYAN (SEQ ID NO:131), (ii) a VL CDR2 comprising the amino acid sequence GTNKRAP (SEQ ID NO:132), (iii) a VL CDR3 comprising the amino acid sequence ALWYSNLWV (SEQ ID NO:133), wherein the amino acid sequence of VL1 is at least 90% identical, at least 91%, at least 92%, at least 93%, at least 94%, at least 95%, at least 96%, at least 97%, at least 98%, or at least 99% identical to SEQ ID NO:135.

In some of these aspects, when the VH1 comprises (i) a VH CDR1 comprising the amino acid sequence TYAMN (SEQ ID NO:128), (ii) a VH CDR2 comprising the amino acid sequence RIRSKYNNYATYYADSVKD (SEQ ID NO:129) and (iii) a VH CDR3 comprising the amino acid sequence HGNFGNSYVSWFAY (SEQ ID NO:130); and the VL1 comprises (i) a VL CDR1 comprising the amino acid sequence RSSTGAVTTSNYAN (SEQ ID NO:131), (ii) a VL CDR2 comprising the amino acid sequence GTNKRAP (SEQ ID NO:132), (iii) a VL CDR3 comprising the amino acid sequence ALWYSNLWV (SEQ ID NO:133), the MM1 comprises the amino acid sequence of SEQ ID NO:72.

The EGFR targeting domain comprises a VH2 (disposed within the 1^{st} polypeptide) and a VH1 (disposed within the second polypeptide). The VH2 comprises a variable heavy chain CDR1 (VH CDR1, also referred to herein as CDRH1), a variable heavy chain CDR2 (VH CDR2, also referred to herein as CDRH2), and a variable heavy chain CDR3 (VH CDR3, also referred to herein as CDRH3), the VL2 comprises a variable light chain CDR1 (VL CDR1, also referred to herein as CDRL1), a variable light chain CDR2 (VL CDR2, also referred to herein as CDRL2), and a variable light chain CDR3 (VL CDR3, also referred to herein as CDRL3).

In some aspects of the present disclosure, the EGFR-targeting heavy chain variable domain (VH2) comprises: (i) a VH CDR1 comprising the amino acid sequence NYGVH (SEQ ID NO:15), (ii) a VH CDR2 comprising the amino acid sequence VIWSGGNTDYNTPFTS (SEQ ID NO:16), and (iii) a VH CDR3 comprising the amino acid sequence ALTYYDYEFAY (SEQ ID NO:17).

In some aspects of the present disclosure, VH2 comprises: (i) a VH CDR1 comprising the amino acid sequence NYGVH (SEQ ID NO:15), (ii) a VH CDR2 comprising the amino acid sequence VIWSGGNTDYNTPFTS (SEQ ID NO:16), and (iii) a VH CDR3 comprising the amino acid sequence ALTYYDYEFAY (SEQ ID NO:17, wherein the amino acid sequence of VH2 that is at least 90% identical, at least 91%, at least 92%, at least 93%, at least 94%, at least 95%, at least 96%, at least 97%, at least 98%, or at least 99% identical to SEQ ID NO:21

In some aspects of the present disclosure, VH2 comprises the amino acid sequence of SEQ ID NO:21.

In certain specific aspects of the present disclosure, the activatable HBPC comprises a first polypeptide comprising an MM1 having the amino acid sequence of SEQ ID NO:1; a VH1 having a VH CDR1 comprising the amino acid sequence of SEQ ID NO:3, a VH CDR2 comprising the amino acid sequence of SEQ ID NO:4, and a VH CDR3 comprising the amino acid sequence of SEQ ID NO:5; a VL1 having a VL CDR 1 having the amino acid sequence of SEQ ID NO:6, a VL CDR2 comprising the amino acid sequence of SEQ ID NO:7, and a VL CDR3 having the amino acid sequence of SEQ ID NO:8; and a VH2 comprising a VH CDR1 comprising the amino acid sequence of SEQ ID NO:15, a VH CDR2 comprising the amino acid sequence of SEQ ID NO:16, a VH CDR3 comprising the amino acid sequence of SEQ ID NO:17. In some of these activatable HBPCs, the second polypeptide comprises an MM2 having the amino acid sequence of SEQ ID NO:13 and a VL2 comprising a VL CDR1 comprising the amino acid sequence of SEQ ID NO:18, a VL CDR2 comprising the amino acid sequence of SEQ ID NO:19, and a VL CDR3 comprising the amino acid sequence of SEQ ID NO:20.

In another specific aspect of the present disclosure, the activatable HBPC comprises a first polypeptide comprising an MM1 having the amino acid sequence of SEQ ID NO:72; a VH1 having a VH CDR1 comprising the amino acid sequence of SEQ ID NO:128, a VH CDR2 comprising the amino acid sequence of SEQ ID NO:129, and a VH CDR3 comprising the amino acid sequence of SEQ ID NO:130; a VL1 having a VL CDR 1 having the amino acid sequence of SEQ ID NO:131, a VL CDR2 comprising the amino acid sequence of SEQ ID NO:132, and a VL CDR3 having the amino acid sequence of SEQ ID NO:133; and a VH2 comprising a VH CDR1 comprising the amino acid sequence of SEQ ID NO:15, a VH CDR2 comprising the amino acid sequence of SEQ ID NO:16, a VH CDR3 comprising the amino acid sequence of SEQ ID NO:17. In some of these activatable HBPCs, the second polypeptide comprises an MM2 having the amino acid sequence of SEQ ID NO:13 and a VL2 comprising a VL CDR1 comprising the amino acid sequence of SEQ ID NO:18, a VL CDR2 comprising the amino acid sequence of SEQ ID NO:19, and a VL CDR3 comprising the amino acid sequence of SEQ ID NO:20.

As described above, the first polypeptide further comprises a monomeric Fc domain (Fc1). Fc domains that are known in the art are suitable for use in the activatable HBPCs of the present disclosure and are described herein below in more detail.

In some aspects of the activatable anti-EGFR, anti-CD3 heteromultimeric bispecific polypeptide described herein, the Fc1 comprises an amino acid sequence that is at least 90% identical, at least 91%, at least 92%, at least 93%, at least 94%, at least 95%, at least 96%, at least 97%, at least 98%, or at least 99% identical to SEQ ID NO:23. In some aspects, the Fc1 comprises the amino acid sequence of SEQ ID NO:23. In certain aspects, the Fc1 comprises the amino acid sequence of SEQ ID NO:24.

In some aspects of the activatable anti-EGFR, anti-CD3 HBPC described herein, the first polypeptide further comprises a heavy chain CH1 domain disposed between the VH2 and the Fc1.

In some aspects of the activatable anti-EGFR, anti-CD3 HBPC described herein, the first polypeptide further comprises an immunoglobulin hinge region disposed between the VH2 and the Fc1. In some aspects where a CH1 domain is present, the immunoglobulin hinge region is disposed between the CH1 domain and the Fc1 domain.

In some aspects of the activatable anti-EGFR, anti-CD3 HBPC described herein, the first polypeptide comprises a structural arrangement from amino-terminus to carboxy-terminus of: MM1-CM1-scFv-VH2-CH1-hinge region-Fc1, wherein each “-” is independently a direct or indirect (e.g., via a linker) linkage.

In some aspects of the activatable anti-EGFR, anti-CD3 HBPC described herein, the first polypeptide further comprises one or more optional linkers, which are described herein below in more detail.

In some aspects of the present disclosure, the activatable anti-EGFR, anti-CD3 HBPC comprises a first polypeptide comprising an Fc1 having the amino acid sequence set forth in SEQ ID NO:23 or SEQ ID NO:24. In some aspects of the present disclosure, an activatable anti-EGFR, anti-CD3 HBPC comprises a first polypeptide comprising a hinge region having the sequence of Hinge-1 (SEQ ID NO:34) or Hinge-2 (SEQ ID NO:35).

In some aspects of the present disclosure, an activatable anti-EGFR, anti-CD3 heteromultimeric bispecific polypeptide comprises a second polypeptide comprising an EGFR-targeting light chain variable domain (VL2) that comprises a VL CDR1, VL CDR2, and VL CDR3.

In some aspects, the present disclosure provides an activatable anti-EGFR, anti-CD3 HBPC comprising a second polypeptide comprising an EGFR-targeting light chain variable domain (VL2) comprising: (i) a CDR1 comprising the amino acid sequence RASQSIGTNIH (SEQ ID NO:18), (ii) a CDR2 comprising the amino acid sequence YASESIS (SEQ ID NO:19), and (iii) a CDR3 comprising the amino acid sequence QQNNNWPTT (SEQ ID NO:20).

In some aspects of the activatable anti-EGFR, anti-CD3 heteromultimeric bispecific polypeptide described herein, the second polypeptide comprises a VL2 having an amino acid sequence that is at least 90% identical, at least 91%, at least 92%, at least 93%, at least 94%, at least 95%, at least 96%, at least 97%, at least 98%, or at least 99% identical to the amino acid sequence of SEQ ID NO:22.

In some aspects of the present disclosure, the VL2 comprises the amino acid sequence set forth in SEQ ID NO:22.

In certain of the above-described aspects of the present disclosure, MM2 comprises the amino acid sequence of SEQ ID NO:13.

In some aspects of the activatable anti-EGFR, anti-CD3 heteromultimeric bispecific polypeptide described herein, the second polypeptide can comprise a structural arrangement from amino-terminus to carboxy-terminus of: MM2-CM2-VL2, wherein each “-” is independently a direct or indirect (e.g., via a linker) linkage.

In some aspects of the activatable anti-EGFR, anti-CD3 heteromultimeric bispecific polypeptide described herein, the second polypeptide comprises one or more linkers. In some aspects, MM2 is joined to CM2 via a linker.

In some aspects, the second polypeptide of the activatable anti-EGFR, anti-CD3 HBPC described herein further comprises a linker comprising between about 1 and about 20 amino acids. Linkers suitable for use in the present disclosure are discussed in more detail below.

In some aspects, the second polypeptide further comprises a constant light chain domain (CL). Exemplary CLs include any of those known in the art. In some aspects, the second polypeptide comprises a CL having the amino acid sequence of SEQ ID NO:25. In certain of these aspects, the second polypeptide comprises a structural arrangement from amino-terminus to carboxy-terminus of: MM2-CM2-VL2-CL, wherein each “-” is independently a direct or indirect (e.g., via a linker) linkage.

In some aspects, the third polypeptide of the activatable HBPC described herein comprises a monomeric Fc domain (Fc2) and does not comprise an immunoglobulin variable domain.

In some aspects, the activatable anti-EGFR, anti-CD3 HBPC disclosed herein comprises a third polypeptide comprising a structural arrangement from amino-terminus to carboxy-terminus of: hinge region-Fc2, wherein each “-” is independently a direct or indirect (e.g., via a linker) linkage. In some aspects, the third polypeptide comprises an Fc2 having an amino acid sequence comprising SEQ ID NO:28 (optionally, with a C-terminal lysine, i.e., SEQ ID NO:29). In one aspect, the third polypeptide comprises a hinge comprising the amino acid sequence of SEQ ID NO:35 and an Fc2 comprising the amino acid sequence of SEQ ID NO:28 (optionally, with a C-terminal lysine, i.e., SEQ ID NO:29). In certain aspects, the first polypeptide comprises a hinge comprising the amino acid sequence of SEQ ID NO:34 and an Fc1 comprising the amino acid sequence of SEQ ID NO:23 (optionally, with a C-terminal lysine, i.e. SEQ ID NO:24).

As provided above, in some aspects, the third polypeptide can comprise a linker, for example between a hinge region and a second Fc domain. The linker can comprise any of the linkers discussed herein.

The activatable anti-EGFR, anti-CD3 HBPCs of the disclosure are activated when the cleavable moiety is cleaved by a protease, thereby generating an activated (i.e., unmasked) anti-EGFR, anti-CD3 heteromultimeric bispecific polypeptide complex (HBPC) that is capable of binding to EGFR and CD3. By comparison, the activatable anti-EGFR, anti-CD3 HBPCs exhibit greatly reduced binding to EGFR and CD3 compared to the activated heteromultimeric bispecific polypeptide because the activatable HBPC remains masked until activated by proteases in the tumor environment. Without wishing to by bound by theory or mechanism, the typical protease activity levels in healthy tissues are likely reduced due to the presence of endogenous inhibitors and/or unfavorable protease pH conditions, while protease activity is generally up-regulated within the tumor environment through up-regulation of protease expression, activation of zymogen, down-modulation of inhibitor expression, or a combination of these effects (See Desnoyers et al., ScienceTranslationalMedicine.org, vol. 5, Issue 207 (October 2013), hereby incorporated by reference).

These activatable anti-EGFR, anti-CD3 HBPCs can therefore be useful in the treatment of a subject having cancer, where proteolytic activity in the tumor microenvironment is upregulated relative to normal tissue and controlled in normal tissues. The greatly reduced binding to EGFR and CD3 of the activatable HBPCs in normal tissue may allow for a reduction in the side effects associated with anti-EGFR and anti-CD3 engagement outside the tumor. In some aspects, the activatable anti-EGFR, anti-CD3 HBPC comprises a first CM and a second CM (CM1 and CM2, respectively).

In some aspects, the CM comprises a substrate for a protease that is upregulated in tumor cells. In some aspects of the HBPC disclosed herein, the CM may comprise a substrate for two or more proteases (i.e., a first protease, a second protease, a third protease, etc.) that are upregulated in tumor cells. There are reports in the literature of increased levels of proteases in a number of cancers, e.g., liquid tumors or solid tumors. See, e.g., La Rocca et al, (2004) British J. of Cancer 90(7): 1414-1421. Numerous studies have demonstrated the correlation of aberrant protease levels, e.g., uPA, legumain, MT-SP1, matrix metalloproteases (MMPs), in solid tumors. (See e.g., Murthy R V, et al. “Legumain expression in relation to clinicopathologic and biological variables in colorectal cancer,” Clin Cancer Res. 11 (2005): 2293-2299; Nielsen B S, et al. “Urokinase plasminogen activator is localized in stromal cells in ductal breast cancer,” Lab Invest 81 (2001): 1485-1501; Look O R, et al. “In situ localization of gelatinolytic activity in the extracellular matrix of metastases of colon cancer in rat liver using quenched fluorogenic DQ-gelatin,” J Histochem Cytochem. 51 (2003): 821-829). ACM may comprise a substrate for multiple proteases, e.g. a substrate for a serine protease and a second different protease, e.g. an MMP. In some aspects, a CM may comprise a substrate for more than one serine protease, e.g., a matriptase and/or uPA. In some aspects, a CM may comprise a substrate for more than one MMP, e.g., MMP9 and MMP14.

In certain embodiments, CM1 and CM2 each independently comprise an amino acid sequence that is a substrate for a protease set forth in Table 1, below.

TABLE 1
Exemplary Proteases

| | | |
|---|---|---|
| ADAMS, | Cysteine | Serine proteases, e.g., |
| ADAMTS, e.g. | proteinases, e.g., | activated protein C |
| ADAM8 | Cruzipain | Cathepsin A |
| ADAM9 | Legumain | Cathepsin G |
| ADAM10 | Otubain-2 | Chymase |
| ADAM12 | KLKs, e.g., | coagulation factor proteases |
| ADAM15 | KLK4 | (e.g., FVIIa, FIXa, FXa, FXIa, |
| ADAM17/TACE | KLK5 | FXIIa) |
| ADAMDEC1 | KLK6 | Elastase |
| ADAMTS1 | KLK7 | Granzyme B |
| ADAMTS4 | KLK8 | Guanidinobenzoatase |
| ADAMTS5 | KLK10 | HtrA1 |
| Aspartate | KLK11 | Human Neutrophil Elastase |
| proteases, e.g., | KLK13 | Lactoferrin |
| BACE | KLK14 | Marapsin |
| Renim | Metallo | NS3/4A |
| Aspartic | proteinases, e.g., | PACE4 |
| cathepsins, e.g., | Meprin | Plasmin |
| Cathepsin D | Neprilysin | PSA |
| Cathepsin E | PSMA | tPA |
| Caspases, e.g., | BMP-1 | Thrombin |
| Caspase 1 | MMPs, | Tryptase |
| Caspase 2 | e.g., | uPA |
| Caspase 3 | MMP1 | Type II Transmembrane |
| Caspase 4 | MMP2 | Serine Proteases |
| Caspase 5 | MMP3 | (TTSPs), e.g., |
| Caspase 6 | MMP7 | DESC1 |
| Caspase 7 | MMP8 | DPP-4 |
| Caspase 8 | MMP9 | FAP |
| Caspase 9 | MMP10 | Hepsin |
| Caspase 10 | MMP11 | Matriptase-2 |
| Caspase 14 | MMP12 | MT-SP1/Matriptase |
| Cysteine | MMP13 | TMPRSS2 |
| cathepsins, e.g., | MMP14 | TMPRSS3 |
| Cathepsin B | MMP15 | TMPRSS4 |
| Cathepsin C | MMP16 | |
| Cathepsin K | MMP17 | |
| Cathepsin L | MMP20 | |
| Cathepsin S | MMP23 | |
| Cathepsin V/L2 | MMP24 | |
| Cathepsin X/Z/P | MMP26 | |
| | MMP27 | |

In some aspects of an activatable anti-EGFR, anti-CD3 HBPC described herein, the CM1 and/or the CM2 includes about three amino acids to about 15 amino acids. In some aspects, the CM1 and/or CM2 may comprise two or more cleavage sites. In some aspects, the two or more cleavage sites on CM1 may comprise a substrate for one protease. In some aspects, the two or more cleavage sites on CM2 may comprise a substrate for two or more proteases. In some aspects, the first protease and the second protease are the same protease. In some aspects, CM1 and CM2 comprise different substrates for the same protease. In some aspects, the CM1 and CM2 comprise the same amino acid sequence. In some aspects, the CM1 and CM2 comprise different amino acid sequences. In some aspects, CM1 comprises the amino acid sequence of SEQ ID NO:73. In some aspects, CM1 comprises the amino acid sequence of SEQ ID NO:2. In some aspects, CM2 comprises the amino acid sequence of SEQ ID NO:14. In certain aspects, the activatable anti-EGFR, anti-CD3 HBPC described herein comprises a CM1 comprising the amino acid sequence of SEQ ID NO:2 and a CM2 comprising the amino acid sequence of SEQ ID NO:14. In some aspects, the activatable anti-EGFR, anti-CD3 heteromultimeric bispecific polypeptide complexes described herein comprise a CM1 that comprises the amino acid sequence of SEQ ID NO:73 and a CM2 the comprises the amino acid sequence of SEQ ID NO:14

Exemplary CMs that are suitable for use in the activatable anti-EGFR, anti-CD3 HBPC described herein include those which are known in the art. Exemplary CMs include but are not limited to those described in, for example, Table 2, and International Publication Nos.: WO 2009/025846, WO 2010/081173, WO 2015/013671, WO 2015/048329, WO 2015/116933, WO 2016/014974, and WO 2016/118629, each of which is incorporated herein by reference in its entirety.

In some aspects, CM1 and/or CM2 comprise an amino acid sequence set forth in Table 2 below. In certain aspects, CM1 and CM2 each independently comprise an amino acid sequence set forth in Table 2 below.

TABLE 2
Cleavable Moieties

| CM SEQ ID NO. | CM SEQUENCE |
|---|---|
| 2 | GLSGRSDDH |
| 14 | ISSGLLSGRSDQH |
| 73 | LSGRSDDH |
| 74 | ISSGLLSGRSDQH |
| 75 | LSGRSDNH |
| 76 | TSTSGRSANPRG |
| 77 | VHMPLGFLGP |
| 78 | AVGLLAPP |
| 79 | QNQALRMA |
| 80 | ISSGLLSS |
| 81 | ISSGLLSGRSDNH |
| 82 | LSGRSGNH |
| 83 | LSGRSDIH |
| 84 | LSGRSDQH |
| 85 | LSGRSDTH |
| 86 | LSGRSDYH |
| 87 | LSGRSDNP |
| 88 | LSGRSANP |
| 89 | LSGRSANI |
| 90 | LSGRSDNI |
| 91 | ISSGLLSGRSANPRG |
| 92 | AVGLLAPPTSGRSANPRG |
| 93 | AVGLLAPPSGRSANPRG |
| 94 | ISSGLLSGRSDDH |
| 95 | ISSGLLSGRSDIH |
| 96 | ISSGLLSGRSDTH |
| 97 | ISSGLLSGRSDYH |
| 98 | ISSGLLSGRSDNP |
| 99 | ISSGLLSGRSANP |
| 100 | ISSGLLSGRSANI |
| 101 | AVGLLAPPGGLSGRSDDH |
| 102 | AVGLLAPPGGLSGRSDIH |
| 103 | AVGLLAPPGGLSGRSDQH |
| 104 | AVGLLAPPGGLSGRSDTH |
| 105 | AVGLLAPPGGLSGRSDYH |
| 106 | AVGLLAPPGGLSGRSDNP |
| 107 | AVGLLAPPGGLSGRSANP |
| 108 | AVGLLAPPGGLSGRSANI |
| 109 | ISSGLLSGRSDNI |
| 110 | AVGLLAPPGGLSGRSDNI |
| ill | ISSGLLSGRSGNH |
| 146 | ALAHGLF |
| 147 | APRSALAHGLF |
| 148 | ISSGLLSGRSNI |
| 149 | LSGRSNI |

In some aspects of the present disclosure, when the activatable HBPC comprises (i) a heavy chain variable domain (VH1) comprising, a VH CDR1 comprising the amino acid sequence KYAMN (SEQ ID NO:3), a VH CDR2 comprising the amino acid sequence RIRSKYNNYATYYADSVKD (SEQ ID NO:4), and a VH CDR3 comprising the amino acid sequence HGNFGNSYISYWAY (SEQ ID NO:5); and a VL1 comprising a VL CDR1 comprising the amino acid sequence GSSTGAVTSGNYPN (SEQ ID NO:6), a VL CDR2 comprising the amino acid sequence GTKFLAP (SEQ ID NO:7), and a VL CDR3 comprising the amino acid sequence VLWYSNRWV (SEQ ID NO:8), the CM1 comprises the amino acid sequence of SEQ ID NO:2. In certain of these activatable HBPCs, MM1 comprises the amino acid sequence of SEQ ID NO:1.

In some aspects of the present disclosure, when the activatable HBPC comprises (i) a heavy chain variable domain (VH1) comprising, a VH CDR1 comprising the amino acid sequence KYAMN (SEQ ID NO:3), a VH CDR2 comprising the amino acid sequence RIRSKYNNYATYYADSVKD (SEQ ID NO:4), and a VH CDR3 comprising the amino acid sequence HGNFGNSYISYWAY (SEQ ID NO:5); and a VL1 comprising a VL CDR1 comprising the amino acid sequence GSSTGAVTSGNYPN (SEQ ID NO:6), a VL CDR2 comprising the amino acid sequence GTKFLAP (SEQ ID NO:7), and a VL CDR3 comprising the amino acid sequence VLWYSNRWV (SEQ ID NO:8), the CM1 comprises the amino acid sequence of SEQ ID NO:73. In certain of these activatable HBPCs, comprises the amino acid sequence of SEQ ID NO:1. In some of these activatable HBPCs, MINH comprises the amino acid sequence of SEQ ID NO:1 and CM1 comprises the amino acid sequence of SEQ ID NO:73.

In a specific aspect of the present disclosure, the activatable HBPC comprises:
(a) a first polypeptide comprising a first heavy chain variable domain (VH1), a first light chain variable domain (VL1), and a second heavy chain variable domain (VH2), a first masking moiety (MM1), a first cleavable moiety (CM1), and a first Fc domain (Fc1),
   wherein the VH1 comprises:
   (i) a VH CDR1 comprising the amino acid sequence KYAMN (SEQ ID NO:3),
   (ii) a VH CDR2 comprising the amino acid sequence RIRSKYNNYATYYADSVKD (SEQ ID NO:4), and
   (iii) a VH CDR3 comprising the amino acid sequence HGNFGNSYISYWAY (SEQ ID NO:5),
   wherein the VL1 comprises:
   (i) a VL CDR1 comprising the amino acid sequence GSSTGAVTSGNYPN (SEQ ID NO:6),
   (ii) a VL CDR2 comprising the amino acid sequence GTKFLAP (SEQ ID NO:7), and
   (iii) a VL CDR3 comprising the amino acid sequence VLWYSNRWV (SEQ ID NO:8);
   wherein the VH2 comprises:
   (i) a VH CDR1 comprising the amino acid sequence NYGVH (SEQ ID NO:15),
   (ii) a VH CDR2 comprising the amino acid sequence VIWSGGNTDYNTPFTS (SEQ ID NO:16), and
   (iii) a VH CDR3 comprising the amino acid sequence ALTYYDYEFAY (SEQ ID NO:17);
(b) a second polypeptide comprising a second light chain variable domain (VL2) and a second masking moiety (MM2) and a second cleavable moiety (CM2),
   wherein the VL2 comprises:
   (i) a VL CDR1 comprising RASQSIGTNIH (SEQ ID NO:18),
   (ii) a VL CDR2 comprising YASESIS (SEQ ID NO:19), and
   (iii) a VL CDR3 comprising QQNNNWPTT (SEQ ID NO:20); and
(c) a third polypeptide comprising a second Fc domain (Fc2),
   wherein the Fc1 binds to Fc2,
   wherein the VH1 and the VL1 together form a targeting domain that specifically binds a CD3 polypeptide,
   wherein the VH2 and the VL2 together form a targeting domain that specifically binds EGFR,
   wherein the third polypeptide does not comprise an immunoglobulin variable domain,
   wherein MM1 is a peptide that interferes with binding of the first targeting domain to the first target,
   wherein MM2 is a peptide that interferes with binding of the second targeting domain to the second target, and
   wherein CM1 and CM2 each independently comprise a substrate for a protease, and wherein the third polypeptide does not comprise an immunoglobulin variable domain. In certain aspects of the present disclosure the VH1 and the VL1 are disposed within an scFv. In some of these aspects, MM1 comprises SEQ ID NO:1, CM1 comprises SEQ ID NO:73, MM2 comprises SEQ ID NO:13, and CM2 comprises SEQ ID NO:14. In some of these aspects, the second polypeptide further comprises a constant light domain (CL) and the third polypeptide further comprises a hinge (HR).

In some aspects of the present disclosure, when the activatable HBPC comprises (i) a VH1 comprising a VH CDR1 comprising the amino acid sequence TYAMN (SEQ ID NO:128), a VH CDR2 comprising the amino acid sequence RIRSKYNNYATYYADSVKD (SEQ ID NO:129) and a VH CDR3 comprising the amino acid sequence HGNFGNSYVSWFAY (SEQ ID NO:130); and (ii) a VL1 comprising a VL CDR1 comprising the amino acid sequence RSSTGAVTTSNYAN (SEQ ID NO:131), a VL CDR2 comprising the amino acid sequence GTNKRAP (SEQ ID NO:132), a VL CDR3 comprising the amino acid sequence of ALWYSNLWV (SEQ ID NO:133), the CM1 comprises the amino acid sequence of SEQ ID NO:73. In some of these activatable HBPCs, MM1 comprises the amino acid sequence of SEQ ID NO:72.

In some of the above-described activatable HBPCs, the first polypeptide further comprises a VH2 having a VH CDR1 comprising the amino acid sequence of SEQ ID NO:15, a VH CDR2 comprising the amino acid sequence of SEQ ID NO:16, a VH CDR3 comprising the amino acid sequence of SEQ ID NO:17. In certain of these activatable HBPCs, the second polypeptide comprises a VL2 comprising a VL CDR1 comprising the amino acid sequence of SEQ ID NO:18, a VL CDR2 comprising the amino acid sequence of SEQ ID NO:19, and a VL CDR3 comprising the amino acid sequence of SEQ ID NO:20. In some of these HBPCs, the third polypeptide comprises the amino acid sequence of SEQ ID NO:28 (and no immunoglobulin variable domain).

In a specific aspect of the present disclosure, the activatable HBPC comprises:
(i) a first polypeptide comprising a first heavy chain variable domain (VH1), a first light chain variable domain (VL1), and a second heavy chain variable domain (VH2), a first masking moiety (MM1), a first cleavable moiety (CM1), and a first Fc domain (Fc1),
wherein the VH1 comprises:
(i) a VH CDR1 comprising the amino acid sequence TYAMN (SEQ ID NO:128),
(ii) a VH CDR2 comprising the amino acid sequence RIRSKYNNYATYYADSVKD (SEQ ID NO:129),
(iii) a VH CDR3 comprising the amino acid sequence HGNFGNSYVSWFAY (SEQ ID NO:130);
wherein the VL1 comprises
(i) a VL CDR1 comprising the amino acid sequence RSSTGAVTTSNYAN (SEQ ID NO:131),
(ii) a VL CDR2 comprising the amino acid sequence GTNKRAP (SEQ ID NO:132), and
(iii) a VL CDR3 comprising the amino acid sequence of ALWYSNLWV (SEQ ID NO:133
wherein the VH2 comprises:
(ii) a VH CDR1 comprising the amino acid sequence NYGVH (SEQ ID NO:15),
(ii) a VH CDR2 comprising the amino acid sequence VIWSGGNTDYNTPFTS (SEQ ID NO:16), and
(iii) a VH CDR3 comprising the amino acid sequence ALTYYDYEFAY (SEQ ID NO:17)
(b) a second polypeptide comprising a second light chain variable domain (VL2) and a second masking moiety (MM2) and a second cleavable moiety (CM2), wherein the VL2 comprises:
   (i) a VL CDR1 comprising RASQSIGTNIH (SEQ ID NO:18),
   (ii) a VL CDR2 comprising YASESIS (SEQ ID NO:19), and
   (iii) a VL CDR3 comprising QQNNNWPTT (SEQ ID NO:20); and
(c) a third polypeptide comprising a second Fc domain (Fc2),
   wherein the Fc1 binds to Fc2,
   wherein the VH1 and the VL1 together form a targeting domain that specifically binds a CD3 polypeptide,
   wherein the VH2 and the VL2 together form a targeting domain that specifically binds EGFR,
   wherein the third polypeptide does not comprise an immunoglobulin variable domain,
   wherein MM1 is a peptide that interferes with binding of the first targeting domain to the first target,
   wherein MM2 is a peptide that interferes with binding of the second targeting domain to the second target, and
   wherein CM1 and CM2 each independently comprise a substrate for a protease, and
   wherein the third polypeptide does not comprise an immunoglobulin variable domain. In certain aspects of the present disclosure the VH1 and the VL1 are disposed within an scFv. In some of these aspects, MM1 comprises SEQ ID NO:72, CM1 comprises SEQ ID NO:73, MM2 comprises SEQ ID NO:13, and CM2 comprises SEQ ID NO:22. In some of these aspects, the second polypeptide further comprises a constant light domain (CL) and the third polypeptide further comprises a hinge (HR).

In some aspects of the activatable anti-EGFR, anti-CD3 HBPCs of the present disclosure, the first polypeptide comprises one or more linkers between the MM and the CM. In some aspects, MM1 is joined to CM1 via a linker. In some aspects, the first polypeptide comprises a linker between the CM1 and the VH2. In certain aspects, the first polypeptide comprises a linker between the VH2 and the Fc1. In some aspects, the first polypeptide comprises at least one linker that is disposed between a pair of elements selected from the group consisting of the MM1 and the CM1; the CM1 and the scFv; the scFv and the VH2; and the VH2 and the Fc1. Linkers suitable for use in the activatable anti-EGFR, anti-CD3 HBPCs described herein are generally ones that provide flexibility of the activatable anti-EGFR, anti-CD3 HBPCs to facilitate the inhibition of the binding of the activatable polypeptide to the target. Such linkers are generally referred to as flexible linkers. Suitable linkers can be readily selected and can be of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, or 20 amino acids in length.

Exemplary flexible linkers include glycine polymers (G)n, glycine-serine polymers (including, for example, (GS)n, (GSGGS)n and (GGGS)n (SEQ ID NO:41 and SEQ ID NO:40 respectfully), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). The ordinarily skilled artisan will recognize that the polypeptides of the HBPC can be designed to include linkers that are all or partially flexible, such that the linker can include a flexible linker as well as one or more portions that confer less flexible structure to provide for a desired structure.

In some aspects, the activatable anti-EGFR, anti-CD3 HBPCs comprise one or more linker sequences disposed in the first, second, and/or third polypeptides. For example in the first polypeptide, a linker is disposed between the MM1 and the CM1, between a heavy chain variable domain and a CH1 domain, between a CH1 domain and a hinge region if both are present, and/or between a hinge region if present and the first Fc domain. In the second polypeptide, which is described elsewhere herein, a linker can be present, for example, between the MM2 and the CM2, between the CM2 and the light chain variable domain, and/or between the light chain variable domain and a CL. In the third polypeptide, which is described elsewhere herein, a linker can be present, for example between a CH1 domain and a second Fc domain, between a CH1 domain and a hinge region, and or between a hinge region and a second Fc domain.

In some aspects of the activatable anti-EGFR, anti-CD3 HBPCs described herein, MM1 is linked to CM1 via linker L1. In some aspects, MM2 is linked to CM2 via linker L2. In some aspects, the amino acid sequence of L1 and L2 are the same. In some aspects, the linker is selected from the group consisting of: (i) a glycine-serine-based linker selected from the group consisting of (GS)n, wherein n is an integer of at least 1, (GGS)n, wherein n is an integer of at least 1 (e.g., an integer from about 1 to about 20, or from about 1 to about 10), (GGGS)n (SEQ ID NO:40), wherein n is an integer of at least 1 (e.g., an integer from about 1 to about 20, or from about 1 to about 10), (GGGGS)n (SEQ ID NO:126), where n is an integer of at least 1 (e.g., an integer from about 1 to about 20, or from about 1 to about 10), (GSGGS)n (SEQ ID NO:41), wherein n is an integer of at least 1 (e.g., an integer from about 1 to about 20, or from about 1 to about 10), GSSGGSGGSG (SEQ ID NO:12), GGSG (SEQ ID NO:42), GGSGG (SEQ ID NO:43), GSGSG (SEQ ID NO:44), GSGGG (SEQ ID NO:45), GGGSG (SEQ ID NO:46), and GSSSG (SEQ ID NO:47), GGGGSGGGGSGGGGSGS (SEQ ID NO:48), GGGGSGS (SEQ ID NO:49), GGGGSGGGGSGGGGS (SEQ ID NO:50), GGGGSGGGGSGGGGSGGGGS (SEQ ID NO:51), GGGGS (SEQ ID NO:52), GGGGSGGGGS (SEQ ID NO:53), GGGS (SEQ ID NO:54), GGGSGGGS (SEQ ID NO:55), GGGSGGGSGGGS (SEQ ID NO:56), GSSGGSGGSGG (SEQ ID NO:57), GGGSGGGGSGGGGSGGGGSGGGGS (SEQ ID NO:58), GGGSSGGS (SEQ ID NO:127) and GS; and (ii) a linker comprising glycine and serine, and at least one of lysine, threonine, or proline selected from the group consisting of GSTSGSGKPGSSEGST (SEQ ID NO:59), SKYGPPCPPCPAPEFLG (SEQ ID NO:60), GGSLDPKGGGGS (SEQ ID NO:61), PKSCDKTHTCPPCPAPELLG (SEQ ID NO:62), GKSSGSGSESKS (SEQ ID NO:63), GSTSGSGKSSEGKG (SEQ ID NO:64), GSTSGSGKSSEGSGSTKG (SEQ ID NO:65), and GSTSGSGKPGSGEGSTKG (SEQ ID NO:66).

In some aspects of the present disclosure, an activatable anti-EGFR, anti-CD3 heteromultimeric bispecific polypeptide can comprise components in addition to those described above. Such components can include a spacer. The term “spacer” refers herein to an amino acid residue or a peptide incorporated at a free terminus of the first, second, and/or third polypeptide. Spacers that are suitable for use in the practice of the present disclosure include any single amino acid residue or any peptide. Suitable spacers include any of those described in, for example, International Publication Nos.: WO 2016/014974, WO 2019/075405, and WO 2019/213444, each of which is incorporated herein by reference in their entireties.

In some aspects, a spacer can comprise from about 1 amino acid to about 10 amino acids (e.g., about 1, 2, 3, 4, 5, 6, 7, 8, or 9 amino acids) or any number there between. In some aspects of an activatable anti-EGFR, anti-CD3 HBPC described herein, the spacer is N-terminally positioned relative to the MM1 and/or MM2. In some aspects, the spacer has a sequence of QGQSGS (SEQ ID NO:116). In some aspects, the spacer has a sequence of QGQSGQG (SEQ ID NO:117). In some aspects, the spacer has a sequence of QGQSGS (SEQ ID NO:118). In some aspects, the spacer has a sequence of QGQSGQG (SEQ ID NO:117).

In some aspects, the Fc domains employed as Fc1 and/or Fc2 are native Fc domains (e.g., a human IgG1 Fc domain or a human IgG4 Fc domain). In some aspects of the present disclosure, the Fc domains employed as Fc1 and/or Fc2 are mutated forms of a native Fc amino acid sequence. The mutations may confer a desired beneficial property to the activatable anti-EGFR, anti-CD3 heteromultimeric bispecific polypeptide (and commensurately, the activated HBPC). For example, certain mutations in the FcRn binding site are known to modulate effector function (see, e.g., Petkova et al., Intl. Immunol. 18:1759-1769, 2006; Deng et al., MAbs 4:101-109, 2012; and Olafson et al., Methods Mol. Biol. 907:537-556, 2012.) The inclusion of any known mutations in an Fc domain that can modulate effector function are suitable. For example, a N297A or N297G mutation in the Fc amino acid sequence may be employed to reduce IgG effector functions (e.g., ADCC and CDC) which may reduce target independent toxicities (see, e.g., Lund et al., Mol. Immunol. 29:35-39, 1992). The Fc domains suitable for use in the context of the present disclosure include any Fc domain known in the art, including but not limited to any known heterodimeric Fc, such as, for example, knob in holes, and the like.

In some aspects, the activatable anti-EGFR, anti-CD3 HBPC disclosed herein further comprises an immunoglobulin hinge region. Suitable hinge regions include any known hinge regions. For example, a hinge region from any the five major classes of immunoglobulins: IgA, IgD, IgE, IgG, and IgM, or subclasses (isotypes) thereof (e.g. IgG1, IgG2, IgG3, IgG4, IgA1 and IgA2) are suitable for use in the present disclosure. The different classes of immunoglobulins have different and well-known subunit structures and three-dimensional configurations.

In some aspects, the first polypeptide Fc1 and the third polypeptide Fc2 hinge regions comprise the same sequence. In some aspects, the first and second Fc domains (Fc1 and Fc2, respectively) of the activatable anti-EGFR, anti-CD3 HBPC described herein are IgG1 Fc domains or IgG4 Fc domains (e.g., a human IgG1 Fc domain or a human IgG4 Fc domain), or variants thereof. In some aspects, Fc1 and/or Fc2 are modified variants of a native (e.g., human) IgG1 Fc domain. In some aspects, Fc 1 and/or Fc2 are modified variants of a native (e.g., human) IgG4 Fc domain.

In some aspects of the activatable anti-EGFR, anti-CD3 HBPC described herein, the Fc1 comprises an amino acid sequence that is at least 90% identical, at least 91%, at least 92%, at least 93%, at least 94%, at least 95%, at least 96%, at least 97%, at least 98%, or at least 99% identical to SEQ ID NO:23. In some aspects, the Fc1 comprises the amino acid sequence of SEQ ID NO:23 (optionally with a C-terminal lysine (i.e., SEQ ID NO:24)).

In some aspects, the third polypeptide further comprises a monomeric Fc domain (Fc2) that binds to Fc1. In some aspects, Fc2 comprises an amino acid sequence that is at least 90%, at least 91%, at least 92%, at least 93%, at least 94%, at least 95%, at least 96%, at least 97%, at least 98%, or at least 99% identical to SEQ ID NO:28. In some aspects, the Fc2 comprises SEQ ID NO:28 (optionally with a terminal lysine (i.e., SEQ ID NO:29)).

In some aspects, the third polypeptide comprises a hinge region having an amino acid sequence selected from the group consisting of SEQ ID NOs: 34 and 35.

As provided elsewhere herein, the format or structure of an activatable anti-EGFR, anti-CD3 HBPC disclosed herein can include any number of optional additional components, including linkers and spacers. By way of example only, the structures set forth below are among the contemplated aspects. However, the aspects shown below are not meant to limit the disclosure in any way.

In some aspects, the activatable anti-EGFR, anti-CD3 heteromultimeric bispecific polypeptide comprises a first polypeptide having a structure (I).

First Polypeptide Structure (I):

(S1) - MM1 - (L1) - CM1 - L2 - VH1 - L3 - VL1 -
(L4) - VH2 - (L5) - (CH11) - (L6) -(Hinge1)-(L7)-
Fc1

wherein
(S1) is an optional spacer;
(L1), (L4), (L5), (L6), and (L7) are each independently an optional linker,
L2 and L3 are linkers,
(CH11) is an optional CH1 domain,
(Hinge1) is an optional hinge region,
the Fc1 is as described hereinabove.

In some aspects, the activatable anti-EGFR, anti-CD3 heteromultimeric bispecific polypeptide comprises a second polypeptide having a structure (II).

Second Polypeptide Structure (II):

(S2) - (L8) - MM2 - (L9) - CM2 -(L10) -VL2 -(CL)

wherein
(S2) is an optional spacer,
(L8), (L9), and (L10) are each independently an optional linker,
MM2 is an anti-EGFR masking moiety, and
VL2 is as described hereinabove; and
(CL) is an optional light chain constant domain.

In some aspects, the activatable anti-EGFR, anti-CD3 heteromultimeric bispecific polypeptide comprises a third polypeptide having a structure (III).

Third Polypeptide Structure (III):

(S3)-(CH12)-(L11)-(Hinge2)-(L12)-Fc2

wherein,
(S3) is an optional spacer,
(CH12) is an optional CH1 domain,
(L11) and (L12) are each independently an optional linker, and
Fc2 is as described hereinabove.

Linkers, spacers, MMs, CMs, Fc domains, CH1 (i.e., CH11 and CH12) domains, hinge regions, and CLs that are suitable for use in structures (I), (II), and (III) include any that are known in the art or that are described herein.

In some aspects of the present disclosure, an activatable anti-EGFR, anti-CD3 HBPC comprises first, second, and third polypeptides, wherein (1) the first polypeptide comprises the amino acid sequence of SEQ ID NO:30 (optionally with a C-terminal lysine and/or optionally without a spacer (e.g., SEQ ID NO:120 (with a terminal lysine and without a spacer)), (2) the second polypeptide comprises the amino acid sequence of SEQ ID NO:31 (or SEQ ID NO:37 (without a spacer)), and (3) the third polypeptide comprises the amino acid sequence of SEQ ID NO:32 (optionally with a C-terminal lysine (i.e., SEQ ID NO:36) (and does not comprise an immunoglobulin variable domain). In some aspects of the present disclosure, an activatable anti-EGFR, anti-CD3 heteromultimeric bispecific polypeptide comprises a first, a second, and a third polypeptide, wherein: (1) the first polypeptide comprises the amino acid sequence of SEQ ID NO:120, (2) the second polypeptide comprises the amino acid sequence of SEQ ID NO:37, and (3) the third polypeptide comprises the amino acid sequence of SEQ ID NO:32 (and does not comprise an immunoglobulin variable domain), as provided in the sequence below.

In some aspects of the present disclosure, an activatable anti-EGFR, anti-CD3 HBPC comprises first, second, and third polypeptides, wherein (1) the first polypeptide comprises the amino acid sequence of SEQ ID NO:30, (2) the second polypeptide comprises the amino acid sequence of SEQ ID NO:31, and (3) the third polypeptide consists of, or consists essentially of the amino acid sequence of SEQ ID NO:32. In some aspects of the present disclosure, an activatable anti-EGFR, anti-CD3 heteromultimeric bispecific polypeptide comprises a first, a second, and a third polypeptide, wherein: (1) the first polypeptide comprises the amino acid sequence of SEQ ID NO:120, (2) the second polypeptide comprises the amino acid sequence of SEQ ID NO:37, and (3) the third polypeptide consists of, or consists essentially of the amino acid sequence of SEQ ID NO:32, and does not comprise an immunoglobulin variable domain, as provided in the sequence below.

In some aspects of the present disclosure, an activatable anti-EGFR, anti-CD3 heteromultimeric bispecific polypeptide comprises a first, a second, and a third polypeptide, wherein: (1) the first polypeptide comprises the amino acid sequence of SEQ ID NO:144, (2) the second polypeptide comprises the amino acid sequence of SEQ ID NO:37, and (3) the third polypeptide consists of, or consists essentially of the amino acid sequence of SEQ ID NO:32, and does not comprise an immunoglobulin variable domain, as provided in the sequence below.

In the first polypeptide shown below, the spacer sequence is in brackets, the mask sequence is underlined, the linkers are bolded, (the linker within the scFv is also italicized and underlined) the substrate (i.e., cleavable moiety) is italicized, and the scFv (which binds a CD3 polypeptide) is italicized and underlined.

### First Polypeptide

(SEQ ID NO: 30)
[QGQSGS]VSTTCWWDPPCTPNTGSSGGSGGSGGLSGRSDDHGGGSEVQL
VESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNF
GNSYISYWAYWGQGTLVTVSS [symbol] [symbol] QTVVTQEPSLT
VSPGGTVTLTCGSSTGAVTSGNYPNWVQQKPGQAPRGLIGGTKFLAPGTP
ARFSGSLLGGKAALTLSGVQPEDEAEYYCVLWYSNRWVFGGGTKLTVLGG
GGSQVQLKQSGPGLVQPSQSLSITCTVSGFSLTNYGVHWVRQSPGKGLEW
LGVIWSGGNTDYNTPFTSRLSINKDNSKSQVFFKMNSLQSQDTAIYYCA
RALTYYDYEFAYWGQGTLVTVSAASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PCEEQYGSTYRCVSVLTVLHQDWLNGKEYKCKVSNKALPAPIEKTISKA
KGQPREPQVYTLPPSRKEMTKNQVSLTCLVKGFYPSDIAVEWESNGQPE
NNYKTTPPVLKSDGSFFLYSKLTVDKSRWQQGNVFSCSVMHEALHNHYT
QKSLSLSPG,

optionally with a C-terminal lysine and/or without spacer (e.g., SEQ ID NO:137). In some aspects, the first polypeptide has the amino acid sequence of SEQ ID NO:120 (without a spacer, but with a C-terminal lysine) or the amino acid sequence of SEQ ID NO:144 (without a spacer and without a C-terminal lysine).

In the second polypeptide depicted below, the spacer sequence is in brackets, the mask sequence is underlined, the linkers are bolded, and the substrate (i.e., cleavable moiety) is italicized.

### Second Polypeptide

(SEQ ID NO: 31)
[QGQSGQG]LSCEGWAMNREQCRAGGGSSGGSISSGLLSGRSDQHGGGSQ
ILLTQSPVILSVSPGERVSFSCRASQSIGTNIHWYQQRTNGSPRLLIKYA
SESISGIPSRFSGSGSGTDFTLSINSVESEDIADYYCQQNNNWPTTFGAG
TKLELKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC,

optionally without spacer (SEQ ID NO:37).

In the third polypeptide depicted below, the hinge region is bolded and underlined, and the remainder of the sequence is the Fc2.

### Third Polypeptide

(SEQ ID NO: 32)
DKTHTCPPCPAPELLGGPSVFLFPPKPKDTLMISRTPEVTCVVVDVSHED
PEVKFNWYVDGVEVHNAKTKPCEEQYGSTYRCVSVLTVLHQDWLNGKEYK
CKVSNKALPAPIEKTISKAKGQPREPQVYTLPPSREEMTKNQVSLTCLVK
GFYPSDIAVEWESNGQPENNYDTTPPVLDSDGSFFLYSDLTVDKSRWQQG
NVFSCSVMHEALHNHYTQKSLSLSPG,

optionally with a C-terminal lysine (SEQ ID NO:36).

### Kits

Provided herein are kits comprising one or more an activatable anti-EGFR, anti-CD3 HBPC described herein or an HBPC thereof, as, wherein the kits are diagnostic or for treatment. In certain aspects, provided herein is a pack or kit comprising one or more containers filled with one or more of the ingredients of the compositions described herein, such as one or more an activatable anti-EGFR, anti-CD3 HBPCs provided herein or an antigen-binding fragment thereof, optional an instructing for use. In some aspects, the kits contain a composition described herein and any diagnostic, prophylactic or therapeutic agent, such as those described herein.

### Therapeutic Uses and Methods

In some aspects, presented herein are methods for treating diseases, e.g., cancers, comprising administering to a subject in need thereof an activatable anti-EGFR, anti-CD3 HBPC, or an HBPC thereof, as described herein, or a pharmaceutical composition thereof as described herein. In some aspects, presented herein are methods of inhibiting tumor growth in a subject in need thereof comprising administering to a subject in need thereof an activatable anti-EGFR, anti-CD3 HBPC, or an HBPC thereof, as described herein, or a pharmaceutical composition thereof as described herein. In some aspects, the present disclosure relates to an activatable anti-EGFR, anti-CD3 HBPC, or an HBPC thereof, as or pharmaceutical composition provided herein for use as a medicament. Usually, the subject is a human, but non-human mammals including transgenic mammals can also be treated.

The amount of an activatable HBPC or HBPC thereof or composition thereof which will be effective in the treatment of a condition will depend on the nature of the disease. The precise dose to be employed in a composition will also depend on the route of administration, and the seriousness of the disease.

Non-limiting examples of disease include: cancers, rheumatoid arthritis, Crohn's disease, SLE, cardiovascular damage, ischemia, etc. For example, indications can include leukemias, including T-cell acute lymphoblastic leukemia (T-ALL), lymphoblastic diseases including multiple myeloma, and solid tumors, including lung, colorectal, prostate, pancreatic and breast, including triple negative breast cancer. For example, indications can include bone disease or metastasis in cancer, regardless of primary tumor origin; breast cancer, including by way of non-limiting example, ER/PR+ breast cancer, Her2+ breast cancer, triple-negative breast cancer; colorectal cancer; endometrial cancer; gastric cancer; glioblastoma; head and neck cancer, such as head and neck squamous cell cancer; esophageal cancer; lung cancer, such as by way of non-limiting example, non-small cell lung cancer; multiple myeloma ovarian cancer; pancreatic cancer; prostate cancer; sarcoma, such as osteosarcoma; renal cancer, such as by way of non-limiting example, renal cell carcinoma; and/or skin cancer, such as by way of non-limiting example, squamous cell cancer, basal cell carcinoma, or melanoma.

### Polynucleotides

In some aspects, provided herein are polynucleotides comprising a nucleotide sequence encoding the first, second, and/or third polypeptide of an activatable anti-EGFR, anti-CD3 HBPC of the present disclosure (correspondingly referred to herein as the “first polynucleotide” the “second polynucleotide”, and the “third polynucleotide”), respectively). Suitable polynucleotides include any that encode any of the first, second, and/or third polypeptides described herein, or portion thereof. An illustrative set of polynucleotide sequences encoding a first, second, and third polypeptide is provided herein below.

Polynucleotides of the present disclosure may be sequence optimized for optimal production from the host organism selected for expression, e.g., by codon/RNA optimization, replacement with heterologous signal sequences, and elimination of mRNA instability elements. Methods to generate optimized nucleic acids encoding an activatable anti-EGFR, anti-CD3 heteromultimeric bispecific polypeptide or antigen-binding fragment thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

### Nucleic Acids

Polynucleotide Encoding a First Polypeptide
(SEQ ID NO: 112)
CAAGGACAATCTGGCTCTGTGTCCACCACCTGTTGGTGGGACCCTCCATG
CACACCTAATACCGGCAGCTCTGGTGGCTCTGGCGGAAGCGGAGGACTGT
CTGGCAGATCCGATGATCACGGCGGAGGATCTGAGGTGCAGCTGGTTGAA
TCTGGTGGCGGACTGGTTCAGCCTGGCGGATCTCTGAAACTGAGCTGTGC
CGCCAGCGGCTTCACCTTCAACAAATACGCCATGAACTGGGTCCGACAGG
CCCCTGGCAAAGGCCTTGAATGGGTCGCCAGAATCAGAAGCAAGTACAAC
AACTATGCCACCTACTACGCCGACAGCGTGAAGGACAGATTCACCATCAG
CCGGGACGACAGCAAGAACACCGCCTACCTGCAGATGAACAACCTGAAAA
CCGAGGACACCGCCGTGTACTACTGTGTGCGGCACGGCAACTTCGGCAAC
AGCTACATCAGCTACTGGGCCTATTGGGGCCAGGGCACACTGGTCACAGT
TTCTAGTGGCGGAGGCGGATCTGGCGGCGGTGGAAGTGGCGGCGGAGGTT
CTCAAACAGTGGTCACCCAAGAGCCTAGCCTGACCGTTTCTCCTGGCGGA
ACCGTGACACTGACATGCGGATCTTCTACAGGCGCCGTGACCAGCGGCAA
CTACCCTAATTGGGTGCAGCAGAAGCCAGGCCAGGCTCCTAGAGGACTGA
TCGGCGGCACAAAGTTTCTGGCTCCCGGAACACCAGCCAGATTCAGCGGT
TCTCTGCTCGGAGGAAAGGCCGCTCTGACACTTTCTGGCGTGCAGCCTGA
GGATGAGGCCGAGTACTATTGCGTGCTGTGGTACAGCAACAGATGGGTGT
TCGGCGGAGGCACCAAGCTGACAGTTCTTGGAGGTGGCGGTAGCCAGGTC
CAGCTGAAACAATCTGGACCCGGACTCGTGCAGCCAAGCCAGAGCCTGTC
TATCACCTGTACCGTGTCCGGCTTCAGCCTGACCAATTACGGCGTGCACT
GGGTTCGACAATCTCCCGGCAAGGGACTCGAATGGCTGGGAGTGATTTGG
AGCGGCGGCAACACCGACTACAACACCCCATTCACCAGCAGACTGAGCAT
CAACAAGGACAACAGCAAGTCCCAGGTGTTCTTCAAGATGAACTCCCTGC
AGAGCCAGGATACCGCCATCTATTACTGCGCTCGGGCCCTGACCTACTAT
GACTACGAGTTTGCCTACTGGGGACAGGGAACCCTCGTGACAGTGTCTGC
TGCTAGCACAAAGGGCCCTAGCGTTTTCCCACTGGCTCCCAGCAGCAAGT
CTACATCCGGTGGAACAGCCGCTCTGGGCTGCCTGGTCAAGGATTACTTT
CCCGAGCCAGTGACCGTGTCCTGGAATAGCGGAGCACTGACATCTGGCGT
GCACACATTTCCAGCCGTGCTGCAGTCTAGCGGCCTGTACTCTCTGTCCA
GCGTTGTGACAGTGCCCAGCAGCTCTCTGGGCACCCAGACCTACATCTGC
AATGTGAACCACAAGCCTAGCAACACCAAGGTGGACAAGAAGGTGGAACC
CAAGAGCTGCGATAAGACACACACCTGTCCTCCATGTCCTGCTCCAGAGC
TGCTCGGAGGCCCTTCCGTGTTTCTGTTCCCTCCAAAGCCTAAGGACACC
CTGATGATCAGCAGAACCCCTGAAGTGACCTGCGTGGTGGTGGATGTGTC
CCACGAGGATCCCGAAGTGAAGTTCAATTGGTACGTCGACGGCGTGGAAG
TGCACAATGCCAAGACCAAGCCTTGCGAGGAACAGTACGGCAGCACCTAC
AGATGCGTGTCCGTGCTGACAGTGCTGCACCAGGATTGGCTGAACGGCAA
AGAGTACAAGTGCAAGGTGTCCAACAAGGCCCTGCCTGCTCCTATCGAGA
AAACCATCAGCAAGGCCAAGGGCCAGCCTAGAGAACCCCAGGTGTACACA
CTGCCTCCAAGCCGGAAAGAGATGACCAAGAATCAGGTGTCCCTGACCTG
CCTGGTCAAGGGCTTCTACCCTTCCGATATCGCCGTGGAATGGGAGAGCA
ATGGACAGCCCGAGAACAACTACAAGACAACCCCTCCTGTGCTGAAGTCC
GACGGCTCATTCTTCCTGTACAGCAAGCTGACCGTGGACAAGAGCAGATG
GCAGCAGGGCAACGTGTTCAGCTGCAGCGTGATGCACGAGGCCCTGCACA
ACCACTACACCCAGAAGTCCCTGTCTCTGAGCCCCGGCAAA

The terminal lysine may not be present in the purified protein regardless of being present or absent in the gene. In a variation of this illustrative polynucleotide, the codon encoding the C-terminal lysine may be absent (i.e., SEQ ID NO:139).

Polynucleotide Encoding a Second Polypeptide
(SEQ ID NO: 113)
CAAGGCCAGTCTGGCCAAGGTCTTAGTTGTGAAGGTTGGGCGATGAATAG
AGAACAATGTCGAGCCGGAGGTGGCTCGAGCGGCGGCTCTATCTCTTCCG
GACTGCTGTCCGGCAGATCCGACCAGCACGGCGGAGGATCCCAAATCCTG
CTGACACAGTCTCCTGTCATACTGAGTGTCTCCCCCGGCGAGAGAGTCTC
TTTCTCATGTCGGGCCAGTCAGTCTATTGGGACTAACATACACTGGTACC
AGCAACGCACCAACGGAAGCCCGCGCCTGCTGATTAAATATGCGAGCGAA
AGCATTAGCGGCATTCCGAGCCGCTTTAGCGGCAGCGGCAGCGGCACCGA
TTTTACCCTGAGCATTAACAGCGTGGAAAGCGAAGATATTGCGGATTATT
ATTGCCAGCAGAACAACAACTGGCCGACCACCTTTGGCGCGGGCACCAAA
CTGGAACTGAAACGTACGGTGGCTGCACCATCTGTCTTCATCTTCCCGCC
ATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGA
CAGCACCTACAGCCTCAGCAGCACCCTGACGCTGAGCAAAGCAGACTACG
AGAAACACAAAGTCTACGCCTGCGAAGTCACCCATCAGGGCCTGAGCTCG
CCCGTCACAAAGAGCTTCAACAGGGGAGAGTGT

The second polypeptide is also encoded by the polynucleotide having the sequence of SEQ ID NO:115.

Polynucleotide Encoding a Third Polypeptide
(SEQ ID NO: 114)
GATAAGACCCACACCTGTCCTCCATGTCCTGCTCCAGAACTGCTCGGCGG
ACCTTCCGTGTTCCTGTTTCCTCCAAAGCCTAAGGACACCCTGATGATCA
GCAGAACCCCTGAAGTGACCTGCGTGGTGGTGGATGTGTCCCACGAGGAT
CCCGAAGTGAAGTTCAATTGGTACGTGGACGGCGTGGAAGTGCACAACGC
CAAGACAAAGCCCTGCGAGGAACAGTACGGCAGCACCTACAGATGCGTGT
CCGTGCTGACAGTGCTGCACCAGGATTGGCTGAACGGCAAAGAGTACAAG
TGCAAGGTGTCCAACAAGGCCCTGCCTGCTCCTATCGAGAAAACCATCAG
CAAGGCCAAGGGCCAGCCTAGAGAACCCCAGGTGTACACACTGCCTCCAA
GCCGGGAAGAGATGACCAAGAACCAGGTGTCCCTGACCTGCCTGGTCAAG
GGCTTCTACCCTTCCGATATCGCCGTGGAATGGGAGAGCAATGGACAGCC
CGAGAACAACTACGACACCACACCTCCAGTGCTGGACAGCGACGGCTCAT
TCTTCCTGTACAGCGACCTGACCGTGGACAAGAGCAGATGGCAGCAGGGC
AACGTGTTCAGCTGCAGCGTGATGCACGAGGCCCTGCACAACCACTACAC
CCAGAAGTCCCTGAGCCTGTCTCCTGGCAAA

In a variation of this illustrative polynucleotide, the codon encoding the C-terminal lysine may be absent (i.e., SEQ ID NO:141).

A further illustrative activatable HBPC of the present disclosure is described in Example 1 that comprises a first polypeptide having the amino acid sequence of SEQ ID NO:30 (encoded by a polynucleotide sequence comprising the polynucleotide sequence of SEQ ID NO:139 or 112 (the terminal lysine is not present in the purified protein regardless of being present or absent in the gene); a second polypeptide having the amino acid sequence of SEQ ID NO:31 (encoded by the polynucleotide sequence of SEQ ID NO:113 or SEQ ID NO:115; and a third polypeptide having the amino acid sequence of SEQ ID NO:32 (encoded by the polynucleotide sequence of SEQ ID NO:114 (the terminal lysine is not present in the purified protein regardless of being present or absent in the gene) or SEQ ID NO:141.

In another illustrative activatable HBPC of the present disclosure, described in Example 1, the activatable HBPC comprises a first polypeptide having the amino acid sequence of SEQ ID NO:38 (encoded by a polynucleotide sequence comprising the polynucleotide sequence of SEQ ID NO:142 or 143 (the terminal lysine is not present in the purified protein regardless of being present or absent in the gene); a second polypeptide having the amino acid sequence of SEQ ID NO:31 (encoded by the polynucleotide sequence of SEQ ID NO:113 or SEQ ID NO:115; and a third polypeptide having the amino acid sequence of SEQ ID NO:32 (encoded by the polynucleotide sequence of SEQ ID NO:114 (the terminal lysine is not present in the purified protein regardless of being present or absent in the gene).or SEQ ID NO:141 (the terminal lysine is not present in the purified protein regardless of being present or absent in the gene).

A polynucleotide encoding a polypeptide or antigen-binding fragment thereof described herein or a domain thereof can be generated from nucleic acid from a suitable source (e.g., a hybridoma) using methods well known in the art (e.g., PCR and other molecular cloning methods), synthesized using techniques that are well known in the art, and the like. Polynucleotides encoding the first, second, and third polypeptides can be cloned into one or more vectors for expression in host cells and for further cloning, for example, to generate chimeric and humanized antibodies or antigen-binding fragments thereof.

Polynucleotides provided herein can be an RNA or a DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

In some aspects, the polynucleotides described herein encode an activatable anti-EGFR, anti-CD3 HBPCs or antigen-binding fragment thereof and comprises the heavy chain (VH) and light chain (VL) and CDRs provided herein.

### Vectors, Host Cells, and Methods of Production

Provided herein are one or more vectors comprising polynucleotides encoding the first, second, and/or third polypeptides of the present disclosure (corresponding to a first polynucleotide, a second polynucleotide, and a third polynucleotide, respectively). In some aspects, such vectors may be used to recombinantly produce the polypeptides of the HBPC from a host cell, as described in more detail hereinbelow. In some aspects, the vector comprises the first, the second, and/or the third polynucleotide operably linked to one or more promoter sequences. In certain aspects, the present disclosure provides a plurality of vectors that collectively comprise the polynucleotides encoding the first, second, and third polypeptides (i.e., the first, second, and third polynucleotides), where the plurality comprises at least one vector that comprises no more than two, or no more than one of the first, the second, and the third polynucleotides. In these aspects, the first, the second, and the third polynucleotide sequences in the plurality of vectors are usually operably linked to one or more promoter sequences.

Also provided herein are recombinant host cells comprising any of the above-described polynucleotides and/or vectors for recombinantly expressing the polynucleotides encoding the polypeptides of the activatable anti-EGFR, anti-CD3 HBPC of the present disclosure. A variety of host-expression vector systems can be utilized to express the polypeptides described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody or antigen-binding fragment thereof described herein in situ. Exemplary host cells that are suitable for use as a recombinant expression host for the above-described polynucleotides include mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20, BMT10 cells, and the like). Vectors employed in the construction of a recombinant mammalian host cell may comprise a promoter derived from the genome of a mammalian cell (e.g., metallothionein promoter) or from a mammalian virus (e.g., the adenovirus late promoter; the vaccinia virus 7.5K promoter). In some aspects, the recombinant host cell is a CHO cell or a NS0 cell.

In some aspects, recombinant expression of a polypeptide described herein, e.g., a first, second, and/or third polypeptide, involves construction of an expression vector containing a polynucleotide that encodes the activatable anti-EGFR, anti-CD3 HBPC. Vector(s) comprising polynucleotides encoding the HBPC can be readily generated by recombinant DNA technology using techniques well known in the art. Methods which are well known to those skilled in the art can be used to construct expression vectors containing one or more polynucleotides encoding that polypeptides described herein, e.g., a first, second, and/or third polypeptide, as well as appropriate transcriptional and translational control signals. These methods include, for example, in vitro recombinant DNA techniques, synthetic techniques, and in vivo genetic recombination. Also provided are replicable vectors comprising a nucleotide sequence operably linked to a promoter. Such vectors can, for example, include the nucleotide sequence encoding the constant region of a polypeptide described herein, e.g., a first, second, and/or third polypeptide (see, e.g., International Publication Nos. WO 86/05807 and WO 89/01036; and U.S. Pat. No. 5,122,464), and variable domains of the polypeptide can be cloned into such a vector for expression of the entire VH, the entire VL, or both the entire VH and VL.

An expression vector can be transferred to a cell (e.g., host cell) by conventional techniques and the resulting cells can then be cultured by conventional techniques to produce the HBPC described herein (e.g., the CDRs, the VH1, VH2, VL1, and VL2 of an activatable anti-EGFR, anti-CD3 heteromultimeric bispecific polypeptide provided herein). Thus, provided herein are host cells containing a polynucleotide encoding the HBPC described herein, operably linked to a promoter for expression of such sequences in the host cell. In some aspects, a host cell contains a vector comprising a polynucleotide encoding the HBPC described herein, or a domain thereof. In some aspects, a host cell contains three different vectors, a first vector comprising a first polynucleotide encoding a first polypeptide described herein, a second vector comprising a second polynucleotide encoding a second polypeptide described herein, and a third vector comprising a third polynucleotide encoding a third polypeptide described herein.

In some aspects, provided herein is a population of vectors that collectively comprise polynucleotides encoding the first, second, and third polypeptide, where each vector comprises only one or two of the polynucleotides encoding the first, second, or third polypeptides. In certain aspects, a single vector is provided herein that comprises the polynucleotides encoding the first, second, and third polypeptides (i.e., the first, second, and third polynucleotides, respectively).

In some aspects, the present disclosure provides methods of producing an activatable HBPC comprising: (a) culturing a host cell comprising one or more polynucleotides encoding the polypeptides of the present disclosure (e.g., a first polynucleotide, a second polynucleotide, and/or a third polynucleotide, as well as vector(s) comprising the aforementioned polynucleotides) in a liquid culture medium under conditions sufficient to produce the activatable HBPC; and (b) recovering the activatable HBPC.

In a particular aspect, provided herein are methods for producing an activatable anti-EGFR, anti-CD3 HBPC, comprising expressing such a polypeptide thereof in a host cell. More specifically, provided herein is a method of producing an activatable HBPC comprising: (a) culturing a host cell comprising one or more polynucleotides encoding the polypeptides of the present disclosure in a liquid culture medium under conditions sufficient to produce the HBPC; and (b) recovering the activatable HBPC.

In another aspect, the method further comprises purifying a bioharvest (cell-free expression product) of activatable HBPC or other in-process composition comprising subjecting an aqueous composition comprising activatable HBPC to a unit operation such as, for example, affinity chromatography, size exclusion chromatography, ion exchange chromatography, ceramic hydroxyapatite chromatography, and the like. In certain aspects, the unit operation is ceramic hydroxyapatite chromatography.

### Compositions

In some aspects, the activatable HBPCs of the present disclosure or HBPC thereof can be utilized in a pharmaceutical composition useful for any of the therapeutic applications disclosed herein. In certain aspects, the pharmaceutical composition comprises a therapeutically effective amount of one or more activatable HBPC, together with pharmaceutically acceptable diluent or carrier. In other aspect, the pharmaceutical composition comprises a therapeutically effective amount of one or more activatable HBPC, a pharmaceutically acceptable diluent, carrier, solubilizer, emulsifier, preservative, and/or adjuvant. Acceptable formulation materials are nontoxic to recipients at the dosages and concentrations employed. The pharmaceutical compositions can be formulated as liquid, frozen or lyophilized compositions.

In certain aspects, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids; antimicrobials; antioxidants; buffers; bulking agents; chelating agents; complexing agents; fillers; carbohydrates such as monosaccharides or disaccharides; proteins; coloring, flavoring and diluting agents; emulsifying agents; hydrophilic polymers; low molecular weight polypeptides; salt-forming counterions (such as sodium); preservatives; solvents (such as glycerin, propylene glycol or polyethylene glycol); sugar alcohols; suspending agents; surfactants or wetting agents; stability enhancing agents; tonicity enhancing agents; delivery vehicles; and/or pharmaceutical adjuvants. Additional details and options for suitable agents that can be incorporated into pharmaceutical compositions are provided in, for example, Remington's Pharmaceutical Sciences, 22^{nd} Edition, (Loyd V. Allen, ed.) Pharmaceutical Press (2013); Ansel et al., Pharmaceutical Dosage Forms and Drug Delivery Systems, 7^{th} ed., Lippencott Williams and Wilkins (2004); and Kibbe et al., Handbook of Pharmaceutical Excipients, 3^{rd} ed., Pharmaceutical Press (2000).

The components of the pharmaceutical composition are selected depending upon, for example, the intended route of administration, delivery format and desired dosage. See, for example, Remington's Pharmaceutical Sciences, 22^{nd} Edition, (Loyd V. Allen, ed.) Pharmaceutical Press (2013). The compositions are selected to influence the physical state, stability, rate of in vivo release and rate of in vivo clearance of the antigen binding proteins disclosed. The primary vehicle or carrier in a pharmaceutical composition can be either aqueous or non-aqueous in nature. For example, a suitable vehicle or carrier can be water for injection or physiological saline solution. In certain aspects, antigen binding protein compositions can be prepared for storage by mixing the selected composition having the desired degree of purity with optional formulation agents in the form of a lyophilized cake or an aqueous solution. Further, in certain aspects, the antigen binding protein can be formulated as a lyophilizate using appropriate excipients.

In certain formulations, the activatable HBPC concentration is at least 2 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml or 150 mg/ml. In other formulations, the activatable HBPC has a concentration of 10-20 mg/ml, 20-40 mg/ml, 40-60 mg/ml, 60-80 mg/ml, or 80-100 mg/ml.

Some compositions include a buffer or a pH adjusting agent. Representative buffers include, but are not limited to: organic acid salts (such as salts of citric acid, acetic acid, ascorbic acid, gluconic acid, carbonic acid, tartaric acid, succinic acid, or phthalic acid); Tris; phosphate buffers; and, in some instances, an amino acid as described below. In certain aspects, buffers are used to maintain the composition at physiological pH or at a slightly lower pH, typically within a pH range of from about 5 to about 8. Some compositions have a pH from about 5-6, 6-7, or 7-8. In other aspects, the pH is from 5.5-6.5, 6.5-7.5, or 7.5-8.5.

Free amino acids or proteins are used in some compositions as bulking agents, stabilizers, and/or antioxidants. As an example, lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant. Glutamine and asparagine are included in some aspects. An amino acid is included in some formulations because of its buffering capacity. Such amino acids include, for instance, alanine, glycine, arginine, betaine, histidine, glutamic acid, aspartic acid, cysteine, lysine, leucine, isoleucine, valine, methionine, phenylalanine, aspartame, and the like. Certain formulations also include a protein excipient such as serum albumin (e.g., human serum albumin (HSA) and recombinant human albumin (rHA)), gelatin, casein, and the like.

Some compositions include a polyol. Polyols include sugars (e.g., mannitol, sucrose, trehalose, and sorbitol) and polyhydric alcohols such as, for instance, glycerol and propylene glycol, and polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations.

Certain compositions include mannitol as a stabilizer. It is generally used with a lyoprotectant, e.g., sucrose. Sorbitol and sucrose are useful for adjusting tonicity and as stabilizers to protect against freeze-thaw stresses during transport or the preparation of bulk product during the manufacturing process. PEG is useful to stabilize proteins and as a cryoprotectant and can be used in the disclosure in this regard.

Sugars, including monosaccharides, di-, tri-, tetra-, and oligosaccharides; derivatized sugars such as alditols, aldonic acids, esterified sugars and the like; and polysaccharides or sugar polymers can be included in some formulations. For example, suitable carbohydrate excipients include, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like.

Surfactants can be included in certain formulations. Surfactants are typically used to prevent, minimize, or reduce protein adsorption to a surface and subsequent aggregation at air-liquid, solid-liquid, and liquid-liquid interfaces, and to control protein conformational stability. Suitable surfactants include, for example, polysorbate 20, polysorbate 80, other fatty acid esters of sorbitan esters, Triton surfactants, lechithin, tyloxapal, and poloxamer 188.

In some aspects, one or more antioxidants are included in the pharmaceutical composition. Antioxidant excipients can be used to prevent oxidative degradation of proteins. Reducing agents, oxygen/free-radical scavengers, and chelating agents are useful antioxidants in this regard. Antioxidants typically are water-soluble and maintain their activity throughout the shelf life of a product. EDTA is another useful antioxidant.

Certain formulations include metal ions that are protein co-factors and that are necessary to form protein coordination complexes. Metal ions also can inhibit some processes that degrade proteins. For example, magnesium ions (10-120 mM) can be used to inhibit isomerization of aspartic acid to isoaspartic acid.

A tonicity enhancing agent can also be included in certain formulations. Examples of such agents include alkali metal halides, preferably sodium or potassium chloride, mannitol, and sorbitol.

One or more preservatives can be included in certain formulations. Preservatives are necessary when developing multi-dose parenteral formulations that involve more than one extraction from the same container. Their primary function is to inhibit microbial growth and ensure product sterility throughout the shelf-life or term of use of the drug product. Suitable preservatives include phenol, m-cresol, p-cresol, o-cresol, chlorocresol, benzyl alcohol, phenylmercuric nitrite, phenoxyethanol, phenyl alcohol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate, thimerosal, benzoic acid, salicylic acid, chlorhexidine, or mixtures thereof in an aqueous diluent.

A pharmaceutical composition is formulated to be compatible with its intended route of administration. Examples of routes of administration are intravenous (IV), intradermal, inhalation, transdermal, topical, transmucosal, and rectal administration.

Formulation components suitable for parenteral administration (e.g., intravenous, subcutaneous, intraocular, intraperitoneal, intramuscular) include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and should be preserved against microorganisms. The carrier can be a solvent or dispersion medium containing, for example, water, ethanol, polyol (for example, glycerol, propylene glycol, and liquid polyethylene glycol), and suitable mixtures thereof.

Further guidance on appropriate formulations depending upon the form of delivery is provided, for example, in Remington's Pharmaceutical Sciences, 22^{nd} Edition, (Loyd V. Allen, ed.) Pharmaceutical Press (2013).

Pharmaceutical formulations can be sterile. Sterilization can be accomplished by any suitable method, e.g., filtration through sterile filtration membranes. Where the composition is lyophilized, filter sterilization can be conducted prior to or following lyophilization and reconstitution. As demonstrated in Examples 7 and 8, the activatable HBPCs described herein appear relatively aggregation-resistant even at relatively high concentrations. Thus, in another aspect, provided herein are compositions comprising any of the activatable HBPCs described herein, and water, wherein the activatable HBPC is present at a concentration of at least 1 mg/mL and wherein the composition comprises at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. As used herein, the term “monomeric activatable HBPC” refers to the activatable HBPC in non-aggregated form. In certain of these aspects the composition comprises at least about 2 mg/ml and at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. In some aspects, the composition comprises at least about 3 mg/ml and at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. In some aspects, the composition comprises at least about 4 mg/ml and at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. The percentage of monomeric activatable HBPC can be readily determined by, for example, size exclusion (SE)-HPLC, as illustrated in Example 7, where percent monomeric activatable HBPC is determined as the percentage peak area corresponding to monomeric activatable HBPC on the basis of total peak area.

### EXAMPLES

The examples in this Examples Section are offered by way of illustration, and not by way of limitation.

### Example 1

### Construction and Expression of Activatable Anti-EGFR, Anti-CD3 Heteromultimeric Bispecific Polypeptides

In this example, two illustrative activatable anti-EGFR, anti-CD3 HBPCs, Complex-57 and Complex-67, were prepared having the structure shown in FIG. 1. With reference to FIG. 1, each of the activatable anti-EGFR, anti-CD3 HBPCs were constructed in three polypeptides as described below:

(a) a first polypeptide including a CD3 masking moiety (MM1) 100, a first cleavable moiety (CM1) 101, an anti-CD3 scFv 102 (including VH1 and VL1 sequences connected via a linker), an anti-EGFR heavy chain variable domain (VH2) (top) and a CH1 domain (bottom), together indicated as 103, which is linked, via a hinge region 109, to a first Fc domain (Fc1) 104; and

(b) a second polypeptide including a EGFR masking moiety (MM2) 105, a second cleavable moiety (CM2) 106, and an anti-EGFR light chain variable domain (VL2) (top) and constant light domain (CL) (bottom), together indicated as 107; and

(c) a third polypeptide including a hinge region 110 and a second Fc domain (Fc2) 108. As seen in FIG. 1, the first and second Fc domains bind each other, and the anti-EGFR heavy and light chain variable domains form an EGFR targeting domain that binds specifically to EGFR. Complex-57 and Complex-67 included the same anti-EGFR targeting domain but included different anti-CD3 scFvs. The components of Complex-67 are listed in Table 4A-4C, and the components of Complex-57 are listed in Tables 5A-5C.

TABLE 4A
Complex-67 First Polypeptide Components

| | First Polypeptide | | | | |
|---|---|---|---|---|---|
| Name | MM1 | CM1 | scFv | VH2 | Fc1^{Δ} |
| First Polypeptide | ML15 | 0011 | i2C | C225v5 | SEQ |
| SEQ ID | SEQ ID | SEQ ID | SEQ ID | SEQ ID | ID |
| NO: 30*^{,++} | NO: 1 | NO: 73 | NO: 11 | NO: 21 | NO: 23 |

*Corresponding polynucleotide sequence is SEQ ID NO: 112 (the terminal lysine is not present in the purified protein regardless of being present or absent in the gene) or SEQ ID NO: 139.
⁺⁺Contains an N-terminal spacer, SEQ ID NO: 33.
^{Δ}Fc1 is located at the C-terminus of a CH1 (SEQ ID NO: 26)-Hinge (SEQ ID NO: 34) sequence.

TABLE 4B
Complex-67 Second Polypeptide Components

| | Second Polypeptide | | | |
|---|---|---|---|---|
| | | | | Constant Light |
| Name | MM2 | CM2 | VL2 | Chain (CL) |
| Second Polypeptide | CF41 | 2008 | C225v5 | CL |
| SEQ ID NO: 31*^{,++} | SEQ ID | SEQ ID | SEQ ID | SEQ ID |
| | NO: 13 | NO: 14 | NO: 22 | NO: 25 |

*Corresponding polynucleotide sequence is SEQ ID NO: 113 or alternatively SEQ ID NO: 115
⁺⁺Contains an N-terminal spacer, SEQ ID NO: 117.

TABLE 4C
Complex-67 Third Polypeptide Components

| | Third Polypeptide |
|---|---|
| Name | Fc2 |
| Third Polypeptide | SEQ ID NO: 28 |
| SEQID NO: 32*^{,++} | |

*Corresponding polynucleotide sequence is SEQ ID NO: 114 (the terminal lysine is not present in the purified protein regardless of being present or absent in the gene) or SEQ ID NO: 141.
⁺⁺Contains a hinge (SEQ ID NO: 35) located at the N-terminus of Fc2.

TABLE 5A
Complex-57 First Polypeptide

| | First Polypeptide | | | | |
|---|---|---|---|---|---|
| Name | MM1 | CM1 | scFv | VH2 | Fc1^{Δ} |
| First Polypeptide | H20GG | 0011 | v16 | C225v5 | SEQ |
| SEQ ID | SEQ ID | SEQ ID | SEQ ID | SEQ ID | ID |
| NO: 38*^{,++} | NO: 72 | NO: 2 | NO: 122 | NO: 21 | NO: 23 |

*Corresponding polynucleotide sequence is SEQ ID NO: 143 (the terminal lysine is not present in the purified protein regardless of being present or absent in the gene) or SEQ ID NO: 142.
⁺⁺Contains an N-terminal spacer (SEQ ID NO: 117).
^{Δ}Fc1 is located at the C-terminus of a CH1 (SEQ ID NO: 26)-Hinge (SEQ ID NO: 34) sequence.

TABLE 5B
Complex-57 Second Polypeptide

| | Second Polypeptide | | | |
|---|---|---|---|---|
| | | | | Constant Light |
| Name | MM2 | CM2 | VL2 | Chain (CL) |
| Second Polypeptide | CF41 | 2008 | C225v5 | CL |
| SEQ ID | SEQ. ID | SEQ ID | SEQ ID | SEQ ID |
| NO: 31*^{,++} | NO: 13 | NO: 14 | NO: 22 | NO: 25 |

* Corresponding polynucleotide sequence is SEQ ID NO: 113 ora ternatively SEQ ID NO: 115
⁺⁺Contains an N-terminal spacer, SEQ ID NO: 117.

TABLE 5C
Complex-57 Third Polypeptide Components

| | Third Polypeptide |
|---|---|
| Name | Fc2 |
| Third Polypeptide | SEQ ID NO: 28 |
| SEQ ID NO: 32*,⁺⁺ | |

*Corresponding polynucleotide sequence is SEQ ID NO: 114 (the terminal lysine is not present in the purified protein regardless of being present in the gene) or SEQ ID NO: 141.
⁺⁺Contains a hinge (SEQ ID NO: 35) located at the N-terminus of Fc2.

### Construction of a Control Activatable Anti-EGFR, Anti-CD3 Heteromultimeric Bispecific Polypeptide

A control activatable bispecific construct, referred to herein as “CI106,” was prepared as described in international patent application Pub. No. WO 2019/075405, which is incorporated herein by reference. CI106 is an activatable dual-armed divalent bispecific construct that is made up of four polypeptides corresponding to two identical heavy chains (two first polypeptides) and to identical light chains (two second polypeptides), where each heavy and light chain form an arm of the bispecific construct. CI106 is “divalent” in that it has two of each type of binding domain (i.e., two EGFR-binding domain and two CD3-binding domains). The amino acid sequence of the light chain is identical to the amino acid sequence of the second polypeptide of Complex-67 and Complex-57. The heavy chain of CI106 and the first polypeptide of Complex-67 have identical spacer, cleavable moieties, anti-EGFR VH, cleavable moiety components. The heavy chain of CI106 and the first polypeptide of Complex-57 have identical spacer, anti-CD3 MM/MM1, cleavable moiety, and anti-CD3 VL/VH (and identical anti-CD3 scFv), and anti-EGFR VH, components. For CI106, all four targeting domains (two anti-CD3 binding domains and two anti-EGFR binding domains) were masked. The components of CI106 are provided in Tables 6A-6B.

TABLE 6A
CI106 Heavy Chain Components

| | Heavy Chain | | | | |
|---|---|---|---|---|---|
| | Anti- | | | Anti-EGFR | |
| | CD3 | | Anti- | Variable | |
| | Masking | Cleavable | CD3 | Heavy | |
| Name | Moiety | Moiety | scFv | Domain | Fc ^{Δ} |
| CI106 | H20GG | 0011 | v16 | C225v5 | SEQ ID |
| SEQ ID | SEQ. ID | SEQ ID | SEQ ID | SEQ ID | NO: 124 |
| NO: 123*^{,++} | NO: 72 | NO: 2 | NO: 122 | NO: 21 | |

*Corresponding polynucleotide sequence is SEQ ID NO: 125 (the protein appears to lose the terminal lysine during expression/purification).
⁺⁺Contains an N-terminal spacer (SEQ ID NO: 116).
^{Δ} The Fc domain is located at the C-terminus of a CH1 (SEQ ID NO:26)-Hinge (SEQ ID NO: 34) sequence.

TABLE 6B
CI106 Light Chain Components

| | | | Light Chain | |
|---|---|---|---|---|
| | Anti-EGFR MM | | Anti-EGFR | Constant |
| Name | (MM2) | CM 2 | VL2 | Light Chain |
| CI106 LC | CF41 | 2008 | C225v5 | CL |
| SEQ ID | SEQ ID | SEQ ID | SEQ ID | SEQ ID |
| NO: 31*^{,++} | NO: 13 | NO: 14 | NO: 22 | NO: 25 |

*Corresponding polynucleotide sequence is SEQ ID NO: 113 or alternatively SEQ ID NO: 115.
⁺⁺Contains an N-terminal spacer, SEQ ID NO: 117.

### Example 2

### Binding of Activatable Anti-EGFR, Anti-CD3 Heteromultimeric Bispecific Polypeptide to EGFR⁺ HT-29 Cells and CD3ε⁺ Jurkat Cells

To assess whether the described anti-EGFR and anti-CD3 masking peptides could inhibit binding of an activatable heteromultimeric bispecific polypeptide to EGFR and CD3, a flow cytometry-based binding assay was performed.

HT-29-luc2 (Perkin Elmer, Inc., Waltham, Mass. (formally Caliper Life Sciences, Inc.) and Jurkat (Clone E6-1, ATCC, TIB-152) cells were cultured in RPMI-1640+glutamax (Life Technologies, Catalog 72400-047) supplemented with 10% Heat Inactivated-Fetal Bovine Serum (HI-FBS, Life Technologies, Catalog 10438-026). As indicated, “activated” (noted herein as “act”) molecules were produced as masked HBPCs and proteolytically cleaved to produce the activated forms. The activatable HBPCs were produced but not subjected to proteolytic cleavage prior to experimentation. The following polypeptide complexes were tested: act-CI106 (a divalent, double-arm bispecific construct), act-Complex-57 (HBPC), and act-Complex-67 (HBPC), and activatable (masked) HBPC Complex-57, (masked) HBPC Complex-67, and dual masked, divalent, double-arm bispecific construct, CI106. As noted in Example 1, one combination of CD3 binder (anti-CD3 scFv v16) and mask (MM H20GG) were utilized in CI106 and Complex-57 and a different combination of CD3 binder (anti-CD3 scFv I2C) and mask (ML15) were utilized in Complex-67.

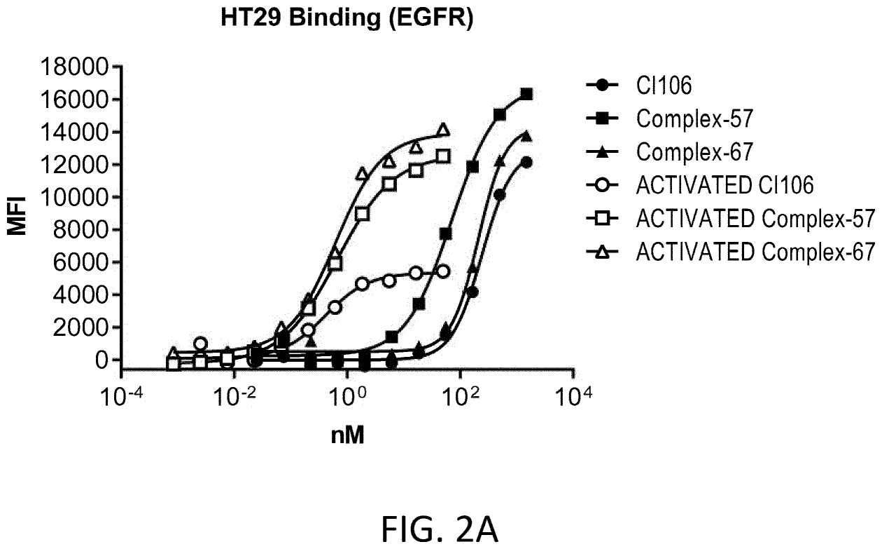

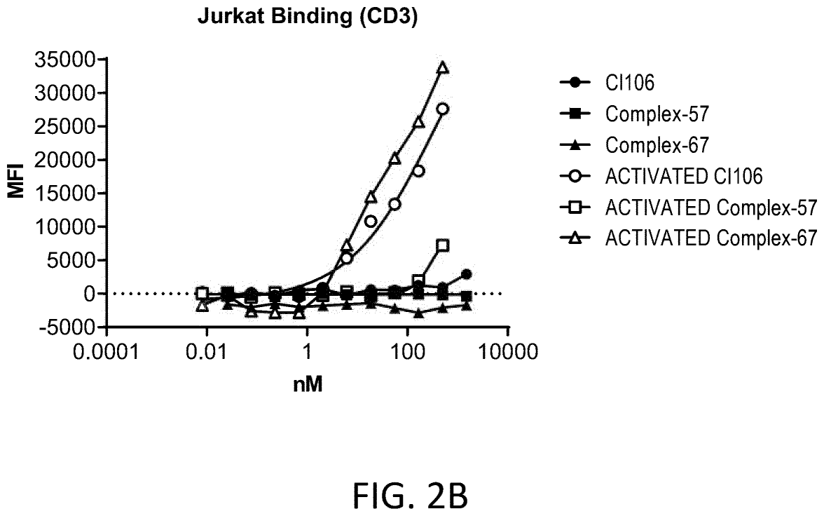

HT29-luc2 cells were detached with Versene™ (Life Technologies, Catalog 15040-066), washed, plated in 96 well plates at approximately 150,000 cells/well, and resuspended in 50 μL of activated or activatable (masked) HBPC. Jurkat cells were counted and plated as described for HT29-luc2 cells. Titrations of activated (unmasked) or activatable (masked) HBPC started at the concentrations indicated in FIGS. 2A and 2B followed by 3-fold serial dilutions in FACS Stain Buffer+2% FBS (BD Pharmingen, Catalog 554656). Cells were incubated at 4° C. with shaking for about 1 hour, harvested, and washed with 2×200 μL of FACS Stain Buffer. Cells were resuspended in 50 μL of Alexa Fluor 488 conjugated anti-Human IgG Fc (10 μg/ml, Jackson ImmunoResearch) and incubated at 4° C. with shaking for about 1 hour. Cells were harvested, washed, and resuspended in a final volume of 200 μL of FACS Stain Buffer containing 2.5 μg/mL 7-AAD (BD Biosciences, Catalog 559925). Cells stained with secondary antibody alone were used as a negative control. Data was acquired on an Attune NxT Flow Cytometer and the median fluorescence intensity (MFI) of viable cells was calculated using FlowJo® V10 (Treestar). Background subtracted MFI data was graphed in GraphPad Prism using curve fit analysis.

As shown in FIGS. 2A-2B, both of the activatable HBPCs, Complex-57 and Complex-67, as well as control CI106 exhibited a reduction in binding to both EGFR and CD3 targets, relative to activated (unmasked) Complex-57, activated Complex-67, and activated CI106. The reduction in binding is represented by a rightward shift of the binding curves. EGFR masking efficiency in this on cell binding experiment was 105 for Complex-57, 338 for Complex-67, and 594 for CI106.

### Example 3

### Biological Activity of Activatable and Activated HBPCs

The biological activity of activatable (masked) and activated (unmasked) HBPCs was assayed using cytotoxicity assays. Human PBMCs were purchased from Stemcell Technologies (Vancouver, Canada) and co-cultured with EGFR expressing cancer cell line HT29-luc2 (Perkin Elmer, Inc., Waltham, Mass. (formally Caliper Life Sciences, Inc.)) at an E (CD3+): T ratio of 5:1 in RPMI-1640+glutamax supplemented with 5% heat inactivated human serum (Sigma, Catalog H3667). Titrations of act-CI106, act-Complex-57 and act-Complex-67, and activatable (masked) CI106, Complex-57 and Complex-67 were tested. After 48 hours, cytotoxicity was evaluated using the ONE-Glo™ Luciferase Assay System (Promega, Madison, Wis. Catalog E6130). Luminescence was measured on the Infinite® M200 Pro (Tecan Trading AG, Switzerland). Percent cytotoxicity was calculated and plotted in GraphPad PRISM with curve fit analysis. Potency of the activated molecules was compared by calculating the EC50. Masking efficiency was calculated as the ratio of intact to activated EC50 for each molecule.

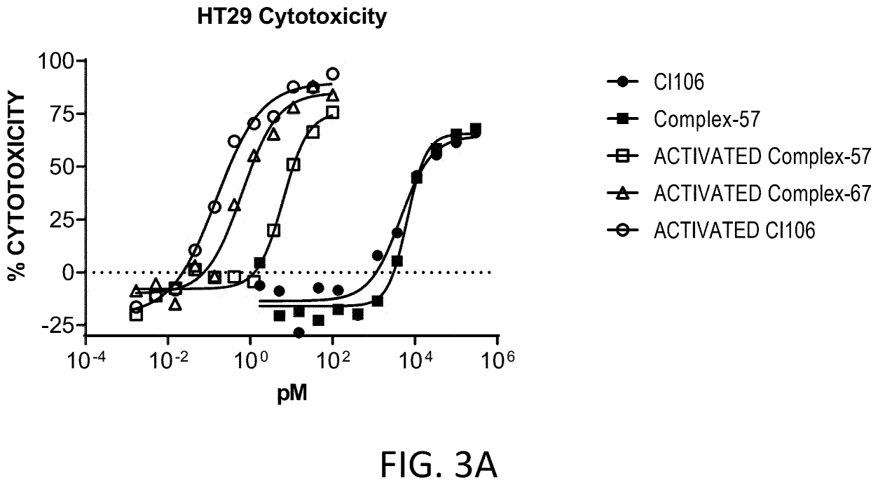

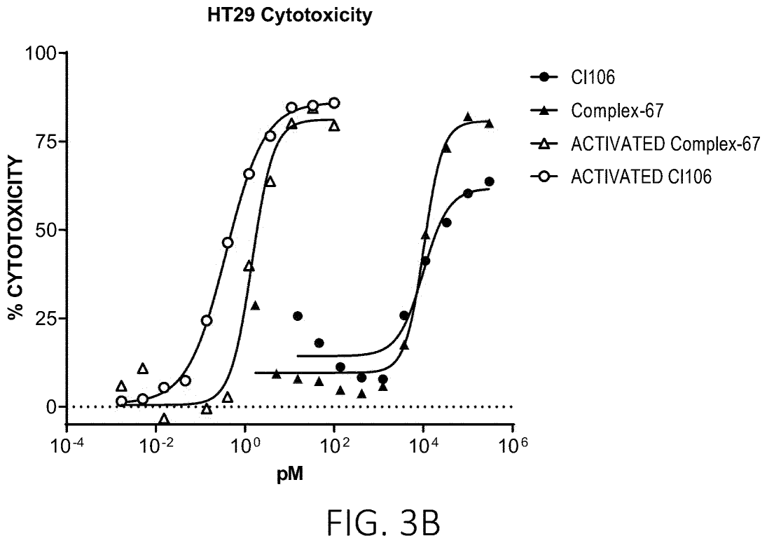

As shown in FIGS. 3A and 3B, the activatable (masked) HBPCs have a shifted dose response curve relative to the activated (unmasked) bispecific antibody. In this assay, the data in FIG. 3A indicates a masking efficiency of 29,650 for CI106 and a masking efficiency of 1,034 for Complex-57. The data in FIG. 3B indicates a masking efficiency of 26,537 for CI106 and a masking efficiency of 7,141 for Complex-67. Complex-57 generally exhibited 10-42 fold reduced potency compared to Complex-67 based on multiple experiments using this assay.

### Example 4

### HBPC Induced Regression of Established HT29 Tumors in Mice

In this example, activatable (masked) HBPC Complex-67 and control CI106, were analyzed for the ability to induce regression or reduce growth of established HT29 xenograft tumors in human PBMC engrafted NSG mice.

The human colon cancer cell line HT29-luc2 (Perkin Elmer, Inc., Waltham, Mass.)) was cultured according to established procedures. Purified, frozen human PBMCs were obtained from Hemacare, Inc. (Van Nuys, Calif.). NSG (NOD.Cg-Prkdcscid Il2rgtm1Wjl/SzJ) mice were obtained from The Jackson Laboratories (Bar Harbor, Me.).

On day 0, each mouse was inoculated subcutaneously at the right flank with 2×10⁶ HT29-luc2 cells in 100 μL RPMI+Glutamax, serum-free medium. Previously frozen PBMCs from a single donor were administered (i.p.) on day 3 at a CD3+ T cell to tumor cell ratio of 1:1. When tumor volumes reached 150-200 mm³ (approximately day 12), mice were randomized into treatment groups and dosed i.v. according to Table 7. Tumor volume and body weights were measured twice weekly. Dose levels of Complex-67 were adjusted to account for molecular weight differences between CI106 and Complex-67.

TABLE 7
Groups and Doses for HT29-luc2 Xenograft Study.

| Group | Count | Treatment | Dose (mg/kg) |
|---|---|---|---|
| 1 | 8 | Vehicle | N/A |
| 2 | 8 | CI106 Dual masked, divalent | 1.0 |
| | | double-armed bispecific | |
| | | construct | |
| 3 | 8 | Complex-67 Activatable | 0.2 |
| | | (masked) HBPC | |
| 4 | 8 | Complex-67 Activatable | 0.6 |
| | | (masked) HBPC | |
| 5 | 8 | Complex-67 Activatable | 1.8 |
| | | (masked) HBPC | |

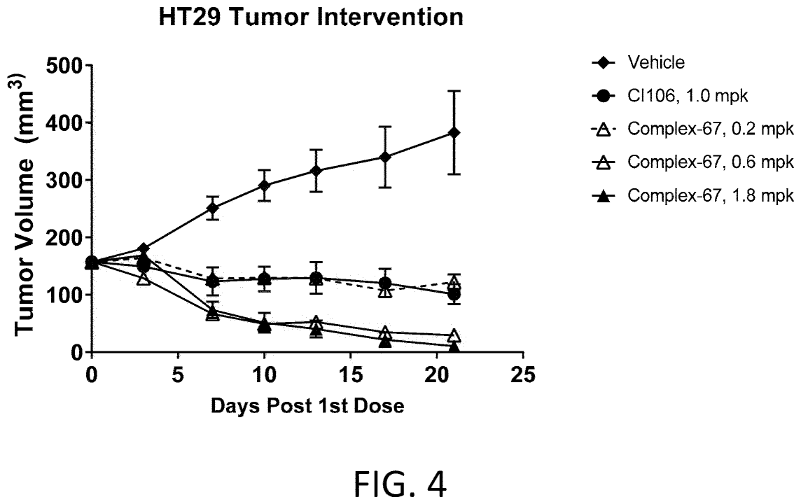

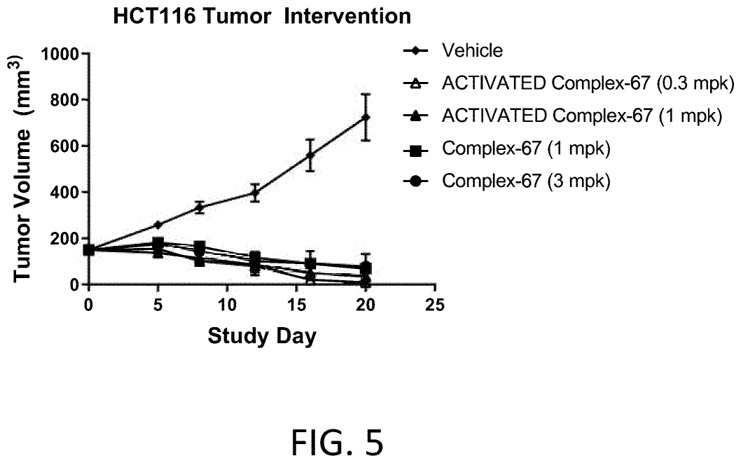

As shown in FIG. 4, which depicts a plot of tumor volume versus days post initial treatment dose (day 0), there is a dose-dependent effect of Complex-67 on the growth of HT29-luc2 xenograft tumors. Complex-67 demonstrated anti-tumor activity that was more potent than the control, CI106, at the equivalent dose (1 mg/kg of CI106 and 0.6 mg/kg of Complex-67); p=0.0099 RMANOVA with Dunnett's).

### Example 5

### Tumor Regression of Established HCT116 Tumors in Mice Following Treatment with Activatable HBPCs

Activated (unmasked) HBPC act-Complex-67, and activatable (masked) HBPC Complex-67 were analyzed for the ability to induce regression or reduce growth of established HCT116 xenograft tumors in human T-cell engrafted NSG mice. The human colon cancer cell line HCT116 (ATCC) was cultured in RPMI+Glutamax+10% FBS according to established procedures. The tumor model was carried out as described in Example 4. Mice were dosed according to Table 8.

TABLE 8
Groups and doses for HCT116 xenograft study.

| Group | Count | Treatment | Dose (mg/kg) |
|---|---|---|---|
| 1 | 8 | Vehicle | N/A |
| 2 | 8 | Complex-67 (Activated, | 0.3 |
| | | unmasked) | |
| 3 | 8 | Complex-67 (Activated, | 1.0 |
| | | unmasked) | |
| 4 | 8 | Complex-67 (Activatable, | 1.0 |
| | | masked) | |
| 5 | 8 | Complex-67 (Activatable, | 3.0 |
| | | masked) | |

### Example 6

### Evaluation of Percent Monomer After Purification with Ceramic Hydroxyapatite Chromatography (CHT)

The dual-masked CI106 control and activatable (masked) HBPC Complex-67 were purified using a ceramic hydroxyapatite chromatography column to compare the amount of dimerization at high concentrations during purification. This was assessed by analyzing the percentage of monomer at each step in the purification process.

Samples were loaded on a CHT Type I, 40 μm bead column (Biorad Cat: 157-0040 and #157-0041) loaded at 20 g/L resin. The column was washed with equilibration buffer 10 mM NaPO4, 100 mM Histidine buffer pH 6.5, then eluted in 2 mL fractions with 10 mM NaPO4, 100 mM Histidine 200 mM Lysine-HCl buffer at pH 6.5 for CI106 and 10 mM NaPO4, 100 mM Histidine 100 mM Lysine-HCl buffer at pH 6.5 for Complex-67. CI106 was collected in 2 mL fractions and then five fractions were pooled to form the eluate. Peak collection started around 25 mAU and stopped around 300 mAU for CI106. Complex-67 was collected in one tube, with peak collection starting at 100 mAU and stopping at 500 mAU. This was followed by a strip buffer step of 500 mM NaPO4 at pH 7.0. Protein concentration for each fraction was quantified by UV absorption at a wavelength of 280 nm. The percent monomer in each fraction was determined by SE-HPLC (Analytical scale size exclusion chromatography) on the basis of total peak area.

During the binding stage of chromatography, protein binds first to the top portion of the column and only moves down the column as the upper sites become full. This causes the molecules to be at a high concentration on the column. The multimeric forms of CI106 and Complex-67 bind with a stronger affinity to the column than the monomeric forms and therefore require a stronger buffer for complete removal from the column. Therefore, when the column is eluted with a weaker buffer and then stripped with a stronger buffer, the eluates have a lower percentage of dimer (higher percentage of monomer) than the strips. As shown in Table 10, the Complex-67 (activatable HBPC) run resulted in an increase in percent monomer of 7.6% in the eluate, leaving the high molecular weight material on the column until the strip step, which led to a 77% recovery in the eluate. This compares to the CI106 run which resulted in a decrease in percent monomer by 5.4% in the eluate to 65.0%, even though more dimeric material (only 30.6% monomer) stayed on the column until the strip, resulting in 81% recovery in the eluate.

TABLE 9
CHT Chromatography Results

| | % | % | % | % | % |
|---|---|---|---|---|---|
| | monomer | Monomer | Recovery | Monomer | Recovery |
| Molecule | in load | in eluate | in eluate | in strip | in strip |
| Complex-67 | 90.9 | 98.5 | 77 | No data | No data |
| CI106 control | 70.4 | 65.0 | 81 | 30.6 | 9 |

These results suggest that Complex-67 does not undergo additional dimerization when at a high concentration on the column, resulting in removal of almost all the high molecular species with 98.5% monomer in the eluate compared to only 65% for CI106. For CI106 there are more high molecular species in the eluate pool than the original load. The improved behavior of Complex-67 enables purification of high monomeric Complex-67 via CHT type 1 chromatography. CI106, however, could not be purified by this, or any bind/elute chromatography method evaluated, due to the dimerization that occurs when CI106 is subjected to high concentrations on the column.

### Example 7

### Assessment of Concentration Dependent Dimerization via Concentrating in a Centrifugal Concentrator

Protein A and SEC-purified preparations of Complex-67, Complex-57 and the dual-masked control CI106 were compared for percent monomer after centrifugal concentration and overnight incubations at the highest concentration.

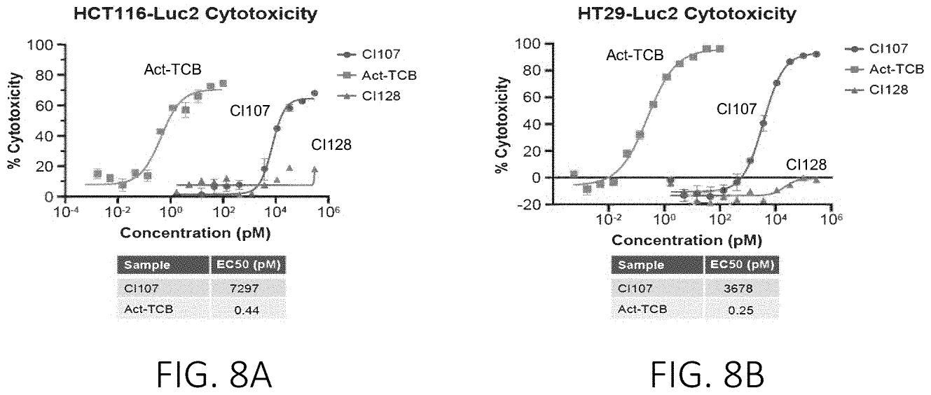

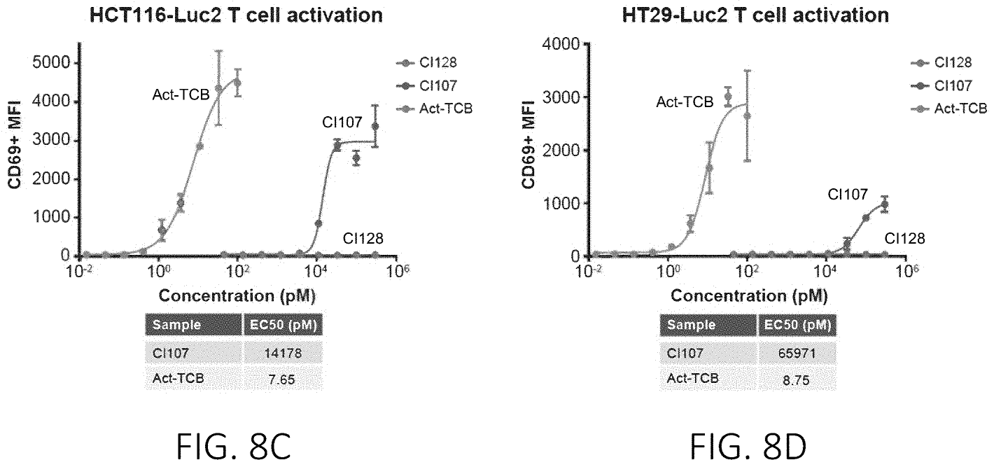

Complex-67, Complex-57 and CI106 were purified with protein A and SEC and then formulated in a low pH buffer (10 mM acetate, 100 mM lysine, pH 6). Samples were diluted 1:15 into PBS (753-45-01) and concentrated using Pierce™ Protein Concentrators PES 10K MWCO 0.5 ml (Thermo Fisher cat #88513) by centrifuging 14,000 RPM for 2 minutes at each concentration. The highest concentrated samples were stored overnight and assessed for percent monomer. The resulting concentrations and percent monomer amounts are shown in Table 10 and FIG. 8.

TABLE 10
Percent Monomeric Activatable HBPC vs. Total Protein Concentration

| Complex-67 | | Complex-57 | | CI106 in PBS | |
|---|---|---|---|---|---|
| Conc. | % | Conc. | % | Conc. | % |
| mg/ml | Monomer | mg/ml | Monomer | mg/ml | Monomer |
| 1.0 | 99.1 | 1.0 | 98.6 | 1.1 | 94.4 |
| 3.2 | 99.0 | 2.4 | 98.4 | 3.58 | 93.0 |
| 5.4 | 98.9 | 3.6 | 98.3 | 5.67 | 90.9 |
| 6.5 | 99.0 | 4.0 | 98.2 | 7.27 | 88.6 |
| Overnight | 98.9 | Overnight | 97.9 | | |

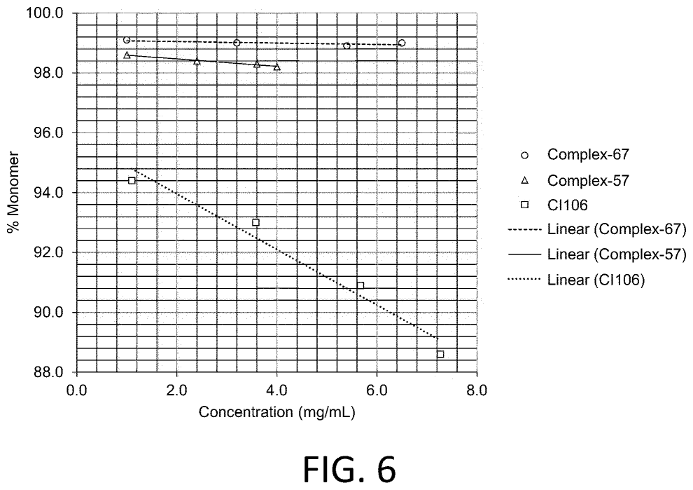

FIG. 6 and Table 10 show that Complex-67 is maintained at a high percentage of monomer (98%-99%) and very low aggregation in solution as concentration is increased. This is in comparison to CI106, which shows a marked concentration dependent dimerization as concentration is increased. Complex-57 showed very little concentration dependent dimerization, maintaining stable monomer percentage as concentration increases. Complex-67 also maintained monomer percentage during an overnight incubation at the highest concentration, demonstrating the stability of the monomer percentage at higher concentration.

### Example 8

### Safety and Efficacy of Activatable Anti-EGFR, Anti-CD3 TCB Construct CI107

In this study, the safety and efficacy of CI107, an anti-EGFR, anti-CD3 TCB construct having the same structural format of the CI106 control (described above), was evaluated in preclinical models to assess the therapeutic potential for the treatment of EGFR-expressing tumors. CI107 was prepared as described in international patent application Pub. No. WO 2019/075405, which is incorporated herein by reference. The CI107 TCB construct is alternatively referred to in this example as a “T cell-engaging bispecific antibody” or “TCB.”

### Methods

### Animal Studies

All animal studies were performed in accordance with the Institutional Animal Care and Use Committee regulations governing the facility that performed each study. Mouse xenograft studies were performed by CytomX Therapeutics, Inc (CytomX), and cynomolgus monkey studies were performed by Altasciences (Everett, Wash.). All animal studies followed regulations set forth by the USDA Animal Welfare Act and the Guide for the Care and Use of Laboratory Animals.

### Materials

All TCBs and other constructs described in this study, including CI107, CI128, CI020, CI011, CI040, CI048, and CI104, were generated by CytomX Therapeutics, Inc. (see, WO 2016/014974 and WO 2019/075405). CI107, CI128, CI020, CI011, CI040, and CI104 have the same structural format as CI106. CI048 corresponds to activated CI011. Activated TCBs were generated by in vitro treatment with urokinase-type plasminogen activator (uPA) followed by SEC purification (Desnoyers 2013). HT29-Luc2 cells were obtained from Caliper Life Sciences (Hopkinton, Mass.), and HCT116 and Jurkat cells were obtained from American Type Culture Collection (ATCC). Human peripheral blood mononuclear cells (PBMCs) were obtained as cryopreserved vials of cells from individual donors from HemaCare Corporation (Northridge, Calif.), AllCells (Alameda, Calif.), or STEMCELL Technologies (Seattle, Wash.). NOD.Cg-Prkcdscid Il2rg tm1Wjl/SzJ (NSG) mice were obtained from Jackson Laboratories (Sacramento, Calif.).

### Cell Binding Assays

HT29 and Jurkat cells were maintained in complete media. HT29 cells were harvested using Versene™ cell dissociation buffer. Cells were centrifuged at 250×g for 5-10 minutes and resuspended in FACS buffer containing 2% FBS (BD Pharminogen). Cells were plated at 150,000/well in V-bottom 96-well plates and treated with Complex-07 or in vitro protease-activated CI104 at various concentrations obtained by 3-fold serial dilutions in FACS buffer, starting at 1.5 μM CI107 for both HT29 and Jurkat cells, 0.05 μM activated CI104 for HT29 cells, and 0.5 μM activated CI104 for Jurkat cells. Cells were incubated for 1 hour at 4° C., washed twice with FACS buffer, and resuspended in 10 μg/ml Alexa Fluor 647 anti-human Fc secondary antibody. The cells were then incubated, protected from light, for 30-60 minutes at 4° C., washed twice with FACS buffer, resuspended in FACS buffer containing 7-AAD, and analyzed on a MACSQuant flow cytometer (Miltenyi Biotech). Mean fluorescence intensity data were corrected for secondary antibody background signal, graphed in Graphpad Prism, and EC50 values were calculated.

### Cytotoxicity Assays

HCT116-Luc2 or HT29-Luc2 were plated into a 96-well white, flat-bottom, tissue culture-treated plate (Costar #3917) at 10,000 cells/well in RPMI+5% human serum. Human PBMCs were freshly thawed and washed twice with RPMI+5% human serum, and 100,000 PBMCs were added in RPMI+5% human serum to the wells containing HCT116-Luc2 or HT29-Luc2. Protease-activated TCB or CI107 was then added to the wells at various concentrations obtained by 3-fold serial dilutions. Control wells contained untreated target+effector cells, target cells only, effector cells only, or media only. The plates were then incubated at 37° C. and 5% CO2 for approximately 48 hours. Cell viability was measured using the ONE-Glo Luciferase Assay System (Promega, #E6120) and a Tecan plate reader. The percent cytotoxicity was calculated as follows: (1-(RLU experimental/average RLU untreated))*100.

### In Vitro T Cell Activation and Cytokine Analysis

T cell activation was measured by induction of CD69 expression in PBMCs co-cultured with HT29-Luc2 or HCT116-Luc2 cells. HT29-Luc2 or HCT116-Luc2 cells were plated at 10,000 cells/well in a U-bottom non-adherent plate. Human PBMCs were freshly thawed and washed twice with RPMI containing serum, and 100,000 PBMCs/well were added to the plates containing tumor cells. Duplicate plates containing PBMCs only were seeded for flow cytometry compensation controls. Three-fold serial dilutions of CI107, activated CI107, or CI128 were prepared in media and added to the plated cells. Cells were incubated at 37° C. and 5% CO2 for 16 hours. To prepare for flow cytometry analysis, plates were centrifuged at 250×g for 10-15 minutes. The supernatant was removed for cytokine analysis, Fc block (Human TruStain FcX, BioLegend) was added to each well, and the plates were incubated for 10 minutes. Antibody cocktails containing anti-CD45-FITC (BioLegend), anti-CD3-Pacific blue (BioLegend), anti-CD8a-APC (BioLegend), and anti-CD69-PE-Cy7 (BioLegend), or appropriate compensation controls were added to the wells, and the plates were incubated with shaking at 4° C. protected from light for 30-60 minutes. The plates were then washed with FACS buffer and resuspended in FACS buffer containing 7-AAD. Fluorescence was measured using an Attune Flow Cytometer, and 15,000 events representing PBMCs were collected.

For cytokine analysis, Meso Scale Discovery U-PLEX plate assays (Meso Scale Diagnostics, Rockville, Mass.) were used. U-PLEX plates were prepared following the manufacturer's protocol to evaluate levels of MCP-1, TNF-α, IL-6, IL-2, and IFN-γ. Supernatant samples collected from HT29-Luc2 or HCT116-Luc2 co-cultured with PBMCs and treated with masked (activatable) CI107, activated (also referred to herein as “act-”) CI107, or CI128 were diluted, added to the plate, and processed following the manufacturer's instructions.

### In Vivo Efficacy Studies

For in vivo experiments, effects of TCBs on tumor growth were measured in mice harboring HT29-Luc2 or HCT116 tumors and engrafted with human T cells resulting from intraperitoneal (IP) injection of human PBMCs. Two million HT29-Luc2 or HCT116 cells were subcutaneously injected in 100 μl serum-free RPMI into the flank of female NSG mice on Day 0. Frozen PBMCs from a single donor were freshly thawed and administered via IP injection on Day 3 in 100-200 μL RPMI+Glutamax, serum-free medium. PBMCs were previously characterized for CD3+ T cell percentage, and the number of PBMCs to be used for in vivo administration was based on a CD3+ T cell to tumor cell ratio of 1:1. Tumor measurements on approximately Day 12 were used to randomize mice prior to intravenous (IV) dosing with TCB, control article, or vehicle. Animals were dosed weekly for 3 weeks with test articles, and tumor volumes and body weights were recorded twice weekly. Activated TCBCI104 was used for in vivo studies. The CI104 construct differs from CI107 only in the cleavable linker used to tether the CD3 mask to the scFv. Upon in vitro protease activation to fully remove the masks, activated CI104 is identical to activated CI107 and can be used to assess the activity of activated CI107, and subsequent in vitro cytotoxicity studies validated that the activity of activated CI104 is the same as that of activated CI107.

### Non-Human Primate Safety Studies

Male cynomolgus monkeys received slow IV bolus injection of test articles on Day 1 or once on Days 1 and 15, depending on the test article. Following test article administration, clinical observations were performed twice daily. Blood samples were collected at various time points post-dose for analysis of cytokine release, serum chemistry, hematology, and toxicokinetics. Cytokine analysis was performed on serum samples using the Life Technologies Monkey Magnetic 29-Plex Panel Kit (Thermo Fisher Scientific, Waltham, Mass.). For toxicokinetic analysis, samples were processed to plasma and stored at −60 to −86° C. prior to shipment for analysis by AIT Bioscience (Indianapolis Ind.) or CytomX. Plasma concentrations of test articles were measured by ELISA using an anti-idiotype capture antibody and an anti-human IgG (Fc) capture antibody. Toxicokinetic analysis was performed by Northwest PK Solutions using a noncompartmental analysis utilizing Phoenix WinNonlin v6.4 (Certara, Princeton, N.J.).

### Results

CI107 was designed as a dual-masked (activatable) dual-armed divalent bispecific molecule containing anti-EGFR and anti-CD3 domains. CI107 was generated using a cetuximab-derived antibody with an SP34-derived anti-CD3ε scFv fused to the N terminus of the heavy chain. CI107 has a human IgG1 Fc domain with mutations that silence Fc function. To generate CI107, a specific masking peptide for the anti-EGFR antibody component was fused to the N terminus of the light chain using a protease-cleavable substrate linker flanked by flexible Gly-Ser-rich peptide linkers, as previously described (Desnoyers 2013). A masking peptide specific for the anti-CD3 component was similarly added to the scFv using a protease-cleavable substrate linker. CI107 impaired Fc-effector function to minimize cross-linking to cells expressing FcγR. The design is intended to maximize target binding and activity in the protease-rich tumor microenvironment while minimizing binding and activity in normal tissues. All of the comparative TCBs used throughout this example contain EGFR and CD3 binding domains, masks, and linker peptides with varying degrees of cleavability. CI011 and CI040 are first generation versions of CI104 and CI107. The CI104 and CI107 molecules contain an optimized CD3 scFv, next generation cleavable linkers, and additional Fc silencing mutations. CI104 and CI107 have the same masks and EGFR and CD3 binding domains, but differ in the CD3 protease linker; however, after protease activation, the activated TCB is the same. CI128 was used as a non-targeted control in which the EGFR binder is replaced by an irrelevant antibody (anti-RSV).

### Masking Impairs Binding to EGFR on the Cell Surface.

To assess whether masking of the EGFR binding domain impairs binding to EGFR expressed on the cell surface, the binding of CI107 and comparative activated TCB constructs (i.e., act-TCBs) to EGFR-expressing HT29 and HCT116 cells was measured.

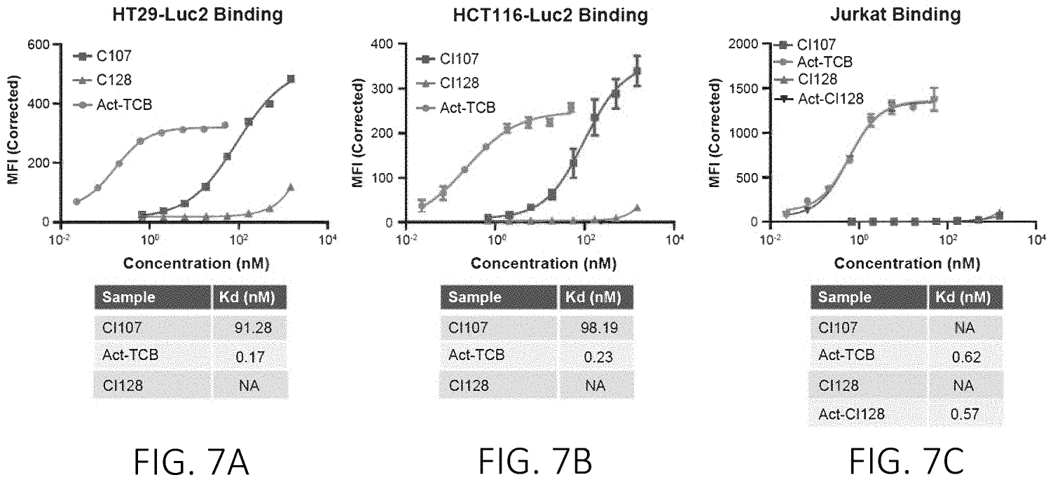

Target cells were incubated with increasing concentrations of CI107 or comparative activated constructs, and binding was evaluated by flow cytometry. As shown in FIGS. 7A and 7B, the presence of the EGFR mask in CI107 substantially attenuated binding to EGFR expressed on the cell surface compared with activated TCB CI107. Activated TCB constructs bound to HT29 cells with a calculated Kd of 0.17 nM, whereas the Kd for binding of CI107 was 91.28 nM, representing a greater than 500-fold decrease in binding compared to activated TCB. Similar results were obtained using HCT116 cells. Binding of CI128, an untargeted control TCB which contains the same anti-CD3 module as CI107 but lacks EGFR targeting was also evaluated. This control did not bind to HT29 or HCT116 cells (see FIGS. 7A and 7B).

### Masking Impairs Binding to CD3 on the Surface of Lymphocytes.

To determine whether masking of the anti-CD3 binding domain impairs binding of CI107 to CD3 on the surface of lymphocytes, CI107 and activated CI107 (i.e., activated TCB) binding to Jurkat cells was measured. As shown in FIG. 7C, activated TCB bound to Jurkat cells with a Kd of 0.62 nM. However, binding of CI107 was not detected, and a Kd could not be calculated. Activated control CI128 bound Jurkat cells with similar affinity as activated TCB.

Together, these data demonstrate that dual masking of anti-EGFR and anti-CD3 binding domains in CI107 attenuates binding to cells expressing EGFR or CD3.

### Masking Attenuates Cytotoxicity and T Cell Activation in PBMCs Co-Culture.

To address whether targeting EGFR with CI107 could lead to anti-tumor cell effects, in vitro cytotoxicity assays were performed. Luciferase-expressing HT29 or HCT116 cells were co-cultured with human PBMCs and incubated with increasing concentrations of CI107, activated TCB, or the untargeted control CI128. After 48 hours of culture, viability of the HCT116-Luc2 or HT29-Luc2 cells was measured via luciferase assay. As shown in FIG. 8A, treatment with the control CI128 resulted in minimal cytotoxicity to HCT116-Luc2 cells co-cultured with PBMCs, demonstrating that engagement of both EGFR and CD3 is required for cytotoxic activity. In contrast, both masked CI107 and activated CI107 (i.e., act-TCB) had cytotoxic effects on HCT116-Luc2 cells. However, activated TCB resulted in cytotoxicity at much lower concentrations compared with the masked form, with EC50 values of 0.44 pM and 7297 pM, respectively. Similar results were observed in HT29-Luc2 cells, with EC50 values of 0.25 pM for activated TCB vs. 3678 pM for CI107 (FIG. 8B). Therefore, dual masking of the anti-EGFR and anti-CD3 domains in CI107 resulted in an approximately 15,000-fold decrease in cytotoxic activity mediated by PBMCs in the absence of protease activation.

Treatment with CI107 Results in Induction of CD69 Expression, a Marker of T Cell Activation.

To determine whether CI107 results in T cell activation, CD69 levels in PBMCs co-cultured with HCT116-Luc2 or HT29-Luc2 cells were measured after treatment with masked CI107, activated CI107 (i.e., act-TCB), and control CI128. CD69 acts as a marker of T cell activation; after TCR/CD3 engagement, CD69 expression is rapidly induced on the surface of T lymphocytes and acts as costimulatory molecule for T cell activation and proliferation. Human PBMCs co-cultured with HCT116-Luc2 or HT29-Luc2 cells were treated with increasing concentrations of CI107, activated TCB (i.e., activated CI107), or control CI128 for 16 hours, and CD69 expression levels were measured by flow cytometry. As shown in FIG. 8C, CI107 resulted in induction of CD69 expression on CD8+ T cells cocultured with HCT116-Luc2 cells with an EC50 of 14178 pM. In contrast, treatment with activated CI107 resulted in CD69 induction with an EC50 of 7.65 pM, reflecting an approximately 18,000-fold shift in the T cell activation curve compared with CI107. T cell activation was not observed with the non-EGFR targeted control CI128, indicating that engagement of CD3 alone is not sufficient for T cell activation. Similarly, treatment of PBMCs from the same donor co-cultured with HT29-Luc2 cells resulted in CD69 induction with EC50 values of 65971 pM for masked CI107 vs. 8.75 pM for activated TCB, reflecting an approximately 7500-fold difference in CD69 induction capacity (FIG. 8D).

Treatment with CI107 Results in Cytokine Release.

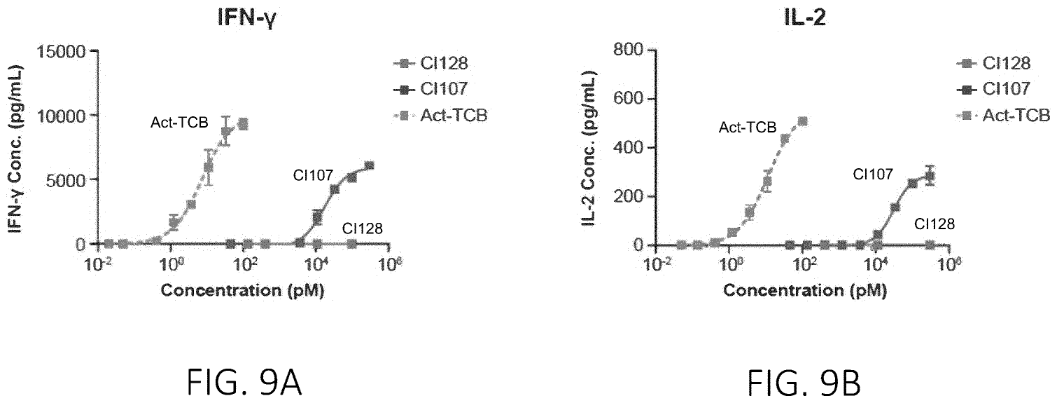

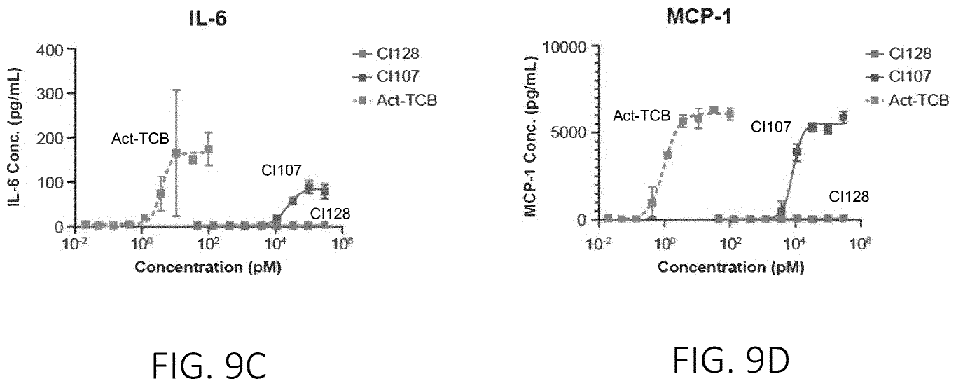

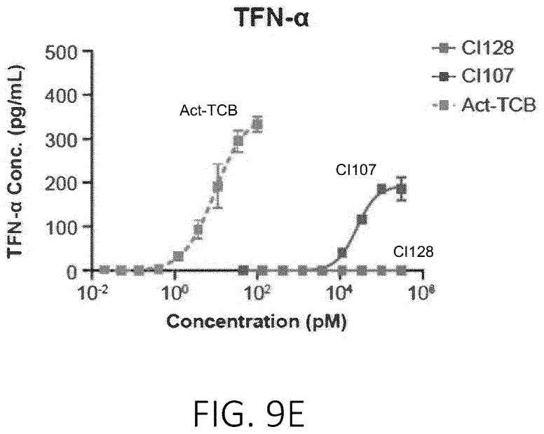

To further assess T cell activation in PBMCs co-cultured with EGFR-expressing cancer cells upon treatment with TCBs, cytokine release was evaluated after treatment with CI107, activated TCB (i.e., activated CI107), or control CI128. Levels of IFN-γ, IL-2, IL-6, MCP-1, and TNF-α were measured 16 hours after treatment with increasing concentrations of TCB. As shown in FIGS. 9A-9E, treatment with CI107 at concentrations in the 104 pM range resulted in release of each of the cytokines measured. In contrast, activated TCB resulted in cytokine release upon treatment with concentrations in the 1-100 pM range. These results were generally consistent between different PBMC donor cells and cancer cell lines (HCT116-Luc2 vs. HT29-Luc2).

Together, these data demonstrate that dual masking of the EGFR and CD3 binding domains in CI107 attenuates T cell activation in the absence of protease activation.

TCB Sensitivity to Protease Cleavage Correlates with In Vivo Anti-Tumor Efficacy and Intratumoral T Cells.

The anti-tumor efficacy of TCBs was evaluated in vivo. Immunocompromised mice harboring HT29-Luc2 tumors and engrafted with human PBMCs were treated once weekly for 3 weeks with vehicle (PBS) or 0.3 mg/kg of TCBs containing linkers with different protease sensitivities (CI011, CI040), a non-cleavable linker (CI020), or the unmasked bispecific therapeutic CI048. CI020 is expected to have minimal anti-tumor activity due to the non-cleavable linker, whereas unmasked CI048 is expected to have maximal efficacy. CI011 and CI040, which both contain EGFR and CD3 masks, have differing protease sensitivities due to different linker peptides; the protease sensitivity of CI040 is greater than that of CI011.

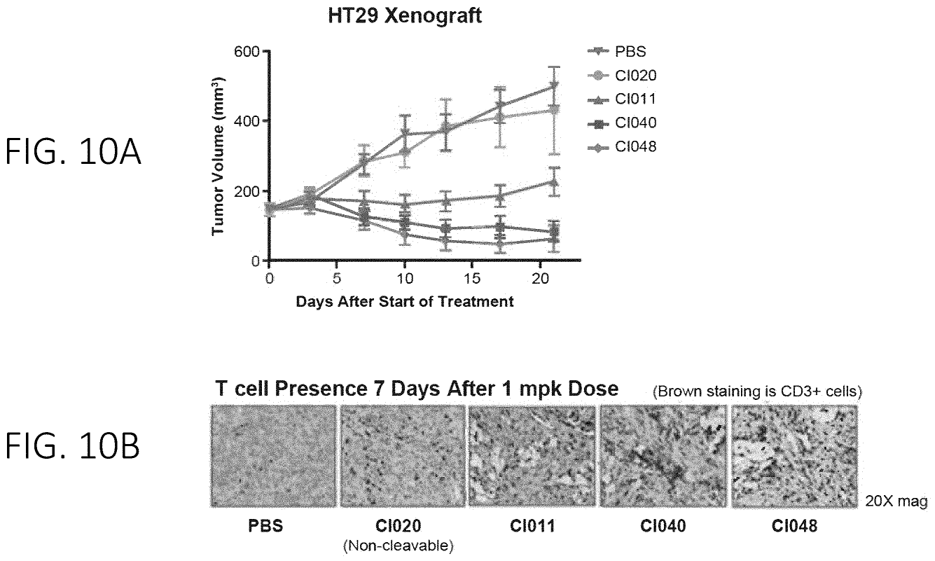

As shown in FIG. 10A, treatment with the unmasked TCB CI048 led to tumor regressions within one week after the start of treatment. Similarly, treatment with masked CI011 and CI040 also resulted in tumor regression or statis; the regression seen with CI040 correlates with the greater cleavability of the linkers in this molecule compared with CI011. In contrast, treatment with CI020, which contains non-cleavable linkers, did not affect tumor growth, indicating that protease cleavability is required for anti-tumor activity of the TCB in vivo.

To determine whether the anti-tumor efficacy mediated by the TCBs tested correlates with T cell presence in the tumors, tumors were harvested one week after animals received a 1 mg/kg dose of masked TCB or activated TCB, and immunohistochemistry for CD3 was performed. As shown in FIG. 10B, minimal numbers of T cells were observed in tumor tissue after treatment with vehicle or the non-cleavable CI020. In contrast, increased numbers of T cells were observed upon treatment with the TCB CI040 or the in vitro protease-activated TCB CI048. Again, the TCB with greater protease sensitivity (CI040) resulted in greater numbers of T cells in the tumor.

Together, these data suggest that TCBs can result in intratumoral T cells and anti-tumor efficacy in vivo that correlates with sensitivity to protease cleavage of the EGFR and CD3 binding domain masks.

Treatment with CI107 Induces Dose-Dependent Regressions of Established Xenograft Tumors.

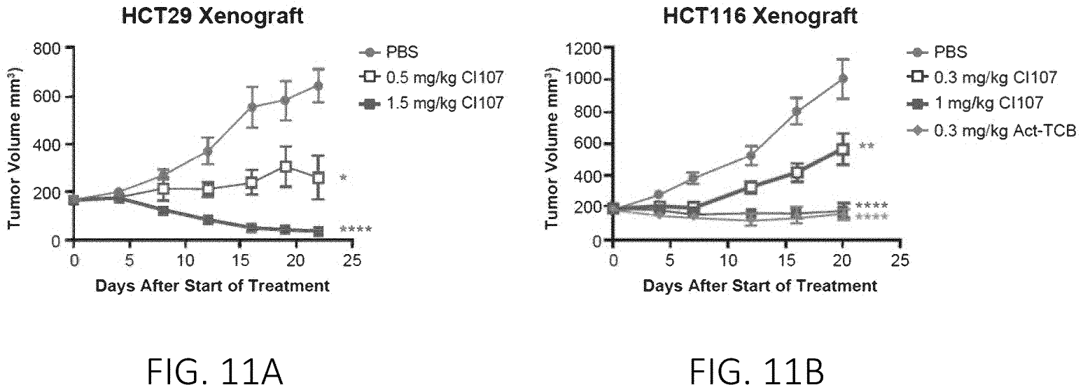

The effects of CI107 on in vivo tumor growth were evaluated. NSG mice were subcutaneously implanted with HT29 cells followed by IP injection of PBMCs, and PBMCs were allowed to engraft for approximately 11 days. Animals were then treated with vehicle, 0.5 mg/kg CI107, or 1.5 mg/kg CI107 once weekly for 3 weeks. As shown in FIG. 11A, treatment with 0.5 mg/kg CI107 resulted in tumor stasis and 1.5 mg/kg CI107 led to tumor regression starting approximately one week after treatment initiation.

The in vivo efficacy of CI107 was also evaluated in HCT116 tumors. After tumor and PBMC engraftment, animals were treated with vehicle, 0.3 mg/kg CI107, 1 mg/kg CI107, or 0.3 mg/kg activated TCB. As shown in FIG. 11B, 0.3 mg/kg CI107 delayed HCT116 tumor growth, whereas 1 mg/kg CI107 and 0.3 mg activated TCB resulted in similar levels of tumor regression and stasis for the duration of treatment.

These data demonstrate that CI107 induces dose-dependent inhibition of tumor growth and regression in HT29 and HCT116 xenograft tumors and that the anti-tumor activity of a 3-fold higher dose of CI107 is similar to that of activated TCB.

### Masked CI107 Provides Increased Safety Relative to Activated CI107 in Cynomolgus Monkeys.

The preclinical tolerability of CI107 was evaluated in cynomolgus monkey studies. Animals received a single administration of 0.06 mg/kg or 0.18 mg/kg activated CI107 (i.e., act-TCB) and 0.6 mg/kg, 2.0 mg/kg, 4.0 mg/kg, or 6.0 mg/kg CI107, and animals were followed for clinical observations. Animals treated with 0.18 mg/kg activated TCB experienced severe clinical effects, including emesis, inappetence, pale appearance, hunched posture, and thin appearance, with adverse effects noted as early as 2 hours and up to 10 days post-dose. Animals treated with 0.06 mg/kg activated TCB experienced moderate and transient clinical effects, including emesis and hunched posture on Day 1 post-dose; based on the rapid resolution of these effects, 0.06 mg/kg was defined as the maximum tolerated dose (MTD) for activated TCB. In contrast, animals treated with 2.0 mg/kg CI107 experienced only transient and mild clinical effects (emesis on Day 2), and animals treated with 0.6 mg/kg CI107 did not experience any adverse effects. Animals treated with 4.0 mg/kg CI107 experienced moderate clinical effects (including emesis at 4, 8, and 24 hours postdose and inappetence on Day 2). The animal treated with 6.0 mg/kg CI107 was found dead on Day 2. Clinical signs noted prior to death included hunched posture, pale appearance, emesis, and liquid feces post dose. Therefore, 4.0 mg/kg was considered the MTD for CI107. Overall, masked CI107 achieved a greater than 60-fold improvement in tolerability compared with activated TCB.

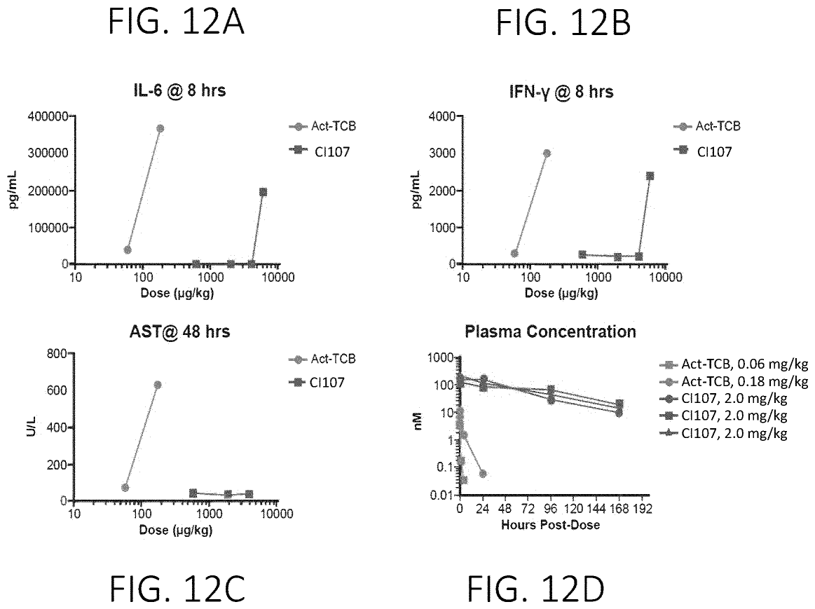

Cytokine levels were also examined after treatment with activated CI107 or masked CI107. As shown in FIG. 12, levels of IL-6 (12A) and IFN-γ (12B) were elevated in animals treated with activated TCB at 8 hours after dosing. In contrast, minimal changes in IL-6 or IFN-γ were observed after treatment with 0.6 mg/kg or 2.0 mg/kg CI107; elevated levels of these cytokines were seen only after treatment with 4.0 mg/kg CI107. Consistent with the clinical observations, CI107 shifts the cytokine release dose-response by more than 60-fold.

Analysis of serum chemistry also demonstrated differences between activated TCB and CI107. As shown in FIG. 12C, treatment with activated TCB led to dose-dependent increases in aspartate aminotransferase (AST), a marker of hepatocellular injury, at 48 hours post-dose. In contrast, no changes in AST were observed after treatment with CI107 at any of the tolerated dose levels, demonstrating improved tolerability with this masked TCB.

To address whether masking of the EGFR and CD3 binding domains affects the pharmacokinetics, the plasma concentrations of activated TCB (i.e., activated CI107) and masked CI107 after dosing were measured. As shown in FIG. 12D, activated TCB was rapidly cleared from circulation within 24 hours after dosing. In contrast, CI107 was maintained in the plasma for up to 7 days after dosing, suggesting that masking may increase exposure relative to the activated TCB. AUC(0-7) following single administration of activated TCB at 0.06 mg/kg was 0.04 day*nM (n=1), while AUC(0-7) following administration of CI107 at 2 mg/kg was 331.7 day*nM (average of n=3), demonstrating a greater than 8,000-fold increase in tolerated exposure.

This demonstrates that improvements in tolerability and pharmacokinetics observed with masked CI107 are consistent with the expected attenuation of binding to EGFR and CD3 in the normal tissue environment.

TABLE 11
Table of Sequences

| SEQ | | |
|---|---|---|
| ID | | |
| NO: | DESCRIPTION | SEQUENCE |
| 1 | MM1 - | VSTTCWWDPPCTPNT |
| | Complex-67 | |
| 2 | CM1 | GLSGRSDDH |
| 3 | VH CDR1 | KYAMN |
| | Complex-67 | |
| 4 | VH CDR2 | RIRSKYNNYATYYADSVKD |
| | Complex-67 | |
| 5 | VH CDR3 | HGNFGNSYISYWAY |
| | Complex-67 | |
| 6 | VL CDR1 | GSSTGAVTSGNYPN |
| | Complex-67 | |
| 7 | VL CDR2 | GTKFLAP |
| | Complex-67 | |
| 8 | VL CDR3 | VLWYSNRWV |
| | Complex-67 | |
| 9 | VH1 | EVQLVESGGGLVQPGGSLKLSCAASGFTFNKYAMNWVRQAPGKGLEW |
| | Complex 67 | VARIRSKYNNYATYYADSVKDRFTISRDDSKNTAYLQMNNLKTEDTAVY |
| | | YCVRHGNFGNSYISYWAYWGQGTLVTVSS |
| 10 | VL1 | QTVVTQEPSLTVSPGGTVTLTCGSSTGAVTSGNYPNWVQQKPGQAPR |
| | Complex-67 | GLIGGTKFLAPGTPARFSGSLLGGKAALTLSGVQPEDEAEYYCVLWYSN |
| | | RVWFGGGTKLTVL |
| 11 | scFv | EVQLVESGGGLVQPGGSLKLSCAASGFTFNKYAMNWVRQAPGKGLEW |
| | Complex-67 | VARIRSKYNNYATYYADSVKDRFTISRDDSKNTAYLQMNNLKTEDTAVY |
| | | YCVRHGNFGNSYISYWAYWGQGTLVTVSSGGGGSGGGGSGGGGSQT |
| | | VVTQEPSLTVSPGGTVTLTCGSSTGAVTSGNYPNWVQQKPGQAPRGLI |
| | | GGTKFLAPGTPARFSGSLLGGKAALTLSGVQPEDEAEYYCVLWYSNRW |
| | | VFGGGTKLTVL |
| 12 | Linker | GSSGGSGGSG |
| 13 | MM2 | LSCEGWAMNREQCRA |
| | Complex-67 | |
| | Complex-57 | |
| 14 | CM2 | ISSGLLSGRSDQH |
| 15 | VH2 CDR1 | NYGVH |
| | Complex-67 | |
| | Complex-57 | |
| 16 | VH2 CDR2 | VIWSGGNTDYNTPFTS |
| | Complex-67 | |
| | Complex-57 | |
| 17 | VH2 CDR3 | ALTYYDYEFAY |
| | Complex-67 | |
| | Complex-57 | |
| 18 | VL2 CDR1 | RASQSIGTNIH |
| | Complex-67 | |
| | Complex-57 | |
| 19 | VL2 CDR2 | YASESIS |
| | Complex-67 | |
| | Complex-57 | |
| 20 | VL2 CDR3 | QQNNNWPTT |
| | Complex-67 | |
| | Complex-57 | |
| 21 | VH2 Domain | QVQLKQSGPGLVQPSQSLSITCTVSGFSLTNYGVHWVRQSPGKGLEWL |
| | Complex-67 | GVIWSGGNTDYNTPFTSRLSINKDNSKSQVFFKMNSLQSQDTAIYYCAR |
| | Complex-57 | ALTYYDYEFAYWGQGTLVTVSA |
| | Cl106 (Control) | |
| 22 | VL2 Domain | QILLTQSPVILSVSPGERVSFSCRASQSIGTNIHWYQQRTNGSPRLLIKYA |
| | Complex-67 | SESISGIPSRFSGSGSGTDFTLSINSVESEDIADYYCQQNNNWPTTFGAG |
| | Complex-57 | TKLELK |
| | Cl106 (Control) | |
| 23 | Fc1 | PAPELLGGPSVFLFPPKPKDTLMISRTPEVTCVVVDVSHEDPEVKFNWY |
| | Complex-67 | VDGVEVHNAKTKPCEEQYGSTYRCVSVLTVLHQDWLNGKEYKCKVSNK |
| | Complex-57 | ALPAPIEKTISKAKGQPREPQVYTLPPSRKEMTKNQVSLTCLVKGFYPSD |
| | | IAVEWESNGQPENNYKTTPPVLKSDGSFFLYSKLTVDKSRWQQGNVFS |
| | | CSVMHEALHNHYTQKSLSLSPG |
| 24 | Fc1 with | PAPELLGGPSVFLFPPKPKDTLMISRTPEVTCVVVDVSHEDPEVKFNWY |
| | terminal lysine | VDGVEVHNAKTKPCEEQYGSTYRCVSVLTVLHQDWLNGKEYKCKVSNK |
| | | ALPAPIEKTISKAKGQPREPQVYTLPPSRKEMTKNQVSLTCLVKGFYPSD |
| | | IAVEWESNGQPENNYKTTPPVLKSDGSFFLYSKLTVDKSRWQQGNVFS |
| | | CSVMHEALHNHYTQKSLSLSPGK |
| 25 | CL Domain | RTVAAPSVFIFPPSDEQLKSGTASVVCLLNNFYPREAKVQWKVDNALQS |
| | Complex-67 | GNSQESVTEQDSKDSTYSLSSTLTLSKADYEKHKVYACEVTHQGLSSPV |
| | Complex-57 | TKSFNRGEC |
| | Cl106 (Control) | |
| 26 | CH1-Human | ASTKGPSVFPLAPSSKSTSGGTAALGCLVKDYFPEPVTVSWNSGALTSG |
| | IgG1 -Complex- | VHTFPAVLQSSGLYSLSSVVTVPSSSLGTQTYICNVNHKPSNTKVDKKV |
| | 67 | |
| | Complex-57 | |
| 27 | BLANK | |
| 28 | Fc2 w/o C- | PAPELLGGPSVFLFPPKPKDTLMISRTPEVTCVVVDVSHEDPEVKFNWY |
| | terminal lysine | VDGVEVHNAKTKPCEEQYGSTYRCVSVLTVLHQDWLNGKEYKCKVSNK |
| | Complex-67 | ALPAPIEKTISKAKGQPREPQVYTLPPSREEMTKNQVSLTCLVKGFYPSD |
| | Complex-57 | IAVEWESNGQPENNYDTTPPVLDSDGSFFLYSDLTVDKSRWQQGNVFS |
| | | CSVMHEALHNHYTQKSLSLSPG |
| 29 | Fc2 with C- | PAPELLGGPSVFLFPPKPKDTLMISRTPEVTCVVVDVSHEDPEVKFNWY |
| | terminal lysine | VDGVEVHNAKTKPCEEQYGSTYRCVSVLTVLHQDWLNGKEYKCKVSNK |
| | | ALPAPIEKTISKAKGQPREPQVYTLPPSREEMTKNQVSLTCLVKGFYPSD |
| | | IAVEWESNGQPENNYDTTPPVLDSDGSFFLYSDLTVDKSRWQQGNVFS |
| | | CSVMHEALHNHYTQKSLSLSPGK |
| 30 | First | [QGQSGS]VSTTCWWDPPCTPNTGSSGGSGGSGGLSGRSDDHGGGSE |
| | Polypeptide | VQLVESGGGLVQPGGSLKLSCAASGFTFNKYAMNWVRQAPGKGLEWV |
| | Complex -67 | ARIRSKYNNYATYYADSVKDRFTISRDDSKNTAYLQMNNLKTEDTAVYY |
| | | CVRHGNFGNSYISYWAYWGQGTLVTVSS [symbol] QTV |
| | | VTQEPSLTVSPGGTVTLTCGSSTGAVTSGNYPNWVQQKPGQAPRGLIG |
| | | GTKFLAPGTPARFSGSLLGGKAALTLSGVQPEDEAEYYCVLWYSNRWV |
| | | FGGGTKLTVLGGGGSQVQLKQSGPGLVQPSQSLSITCTVSGFSLTNYG |
| | | VHWVRQSPGKGLEWLGVIWSGGNTDYNTPFTSRLSINKDNSKSQVFFK |
| | | MNSLQSQDTAIYYCARALTYYDYEFAYWGQGTLVTVSAASTKGPSVFPL |
| | | APSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHT |
| | | CPPCPAPELLGGPSVFLFPPKPKDTLMISRTPEVTCVVVDVSHEDPEVK |
| | | FNWYVDGVEVHNAKTKPCEEQYGSTYRCVSVLTVLHQDWLNGKEYKC |
| | | KVSNKALPAPIEKTISKAKGQPREPQVYTLPPSRKEMTKNQVSLTCLVKG |
| | | FYPSDIAVEWESNGQPENNYKTTPPVLKSDGSFFLYSKLTVDKSRWQQ |
| | | GNVFSCSVMHEALHNHYTQKSLSLSPG |
| 31 | Second | [QGQSGQG]LSCEGWAMNREQCRAGGGSSGGSISSGLLSGRSDQHGG |
| | Polypeptide | GSQILLTQSPVILSVSPGERVSFSCRASQSIGTNIHWYQQRTNGSPRLLI |
| | Complex - 67 | KYASESISGIPSRFSGSGSGTDFTLSINSVESEDIADYYCQQNNNWPTTF |
| | Complex-57 | GAGTKLELKRTVAAPSVFIFPPSDEQLKSGTASVVCLLNNFYPREAKVQ |
| | Cl106 (control) | WKVDNALQSGNSQESVTEQDSKDSTYSLSSTLTLSKADYEKHKVYACE |
| | | VTHQGLSSPVTKSFNRGEC |
| 32 | Third | DKTHTCPPCPAPELLGGPSVFLFPPKPKDTLMISRTPEVTCVVVDVSHE |
| | Polypeptide | DPEVKFNWYVDGVEVHNAKTKPCEEQYGSTYRCVSVLTVLHQDWLNG |
| | Complex - 67 | KEYKCKVSNKALPAPIEKTISKAKGQPREPQVYTLPPSREEMTKNQVSLT |
| | Complex - 57 | CLVKGFYPSDIAVEWESNGQPENNYDTTPPVLDSDGSFFLYSDLTVDKS |
| | | RWQQGNVFSCSVMHEALHNHYTQKSLSLSPG |
| 33 | Spacer | QGQSGS |
| 34 | Hinge-1 | EPKSCDKTHTCPPC |
| | Complex-67 | |
| | Complex-57 | |
| | Cl106 (control) | |
| 35 | Hinge-2 | DKTHTCPPC |
| | Complex-67 | |
| | Complex-57 | |
| 36 | Third | DKTHTCPPCPAPELLGGPSVFLFPPKPKDTLMISRTPEVTCVVVDVSHE |
| | Polypeptide | DPEVKFNWYVDGVEVHNAKTKPCEEQYGSTYRCVSVLTVLHQDWLNG |
| | with terminal | KEYKCKVSNKALPAPIEKTISKAKGQPREPQVYTLPPSREEMTKNQVSLT |
| | lysine | CLVKGFYPSDIAVEWESNGQPENNYDTTPPVLDSDGSFFLYSDLTVDKS |
| | Complex-67 | RWQQGNVFSCSVMHEALHNHYTQKSLSLSPGK |
| | Complex-57 | |
| 37 | Complex-67; | LSCEGWAMNREQCRAGGGSSGGSISSGLLSGRSDQHGGGSQILLTQS |
| | Complex-57 | PVILSVSPGERVSFSCRASQSIGTNIHWYQQRTNGSPRLLIKYASESISGI |
| | 2^{nd} polypeptide | PSRFSGSGSGTDFTLSINSVESEDIADYYCQQNNNWPTTFGAGTKLELK |
| | without spacer | RTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPV |
| | | TKSFNRGEC |
| 38 | Complex-57 | QGQSGSGYLWGCEWNCGGITTGSSGGSGGSGGLSGRSDDHGGGSQ |
| | 1^{st} Polypeptide | TVVTQEPSFSVSPGGTVTLTCRSSTGAVTTSNYANWVQQTPGQAPRGL |
| | w/o terminal | IGGTNKRAPGVPDRFSGSILGNKAALTITGAQADDESDYYCALWYSNLW |
| | lysine. | VFGGGTKLTVLGGGGSGGGGSGGGGSEVQLVESGGGLVQPGGSLKLS |
| | | CAASGFTFSTYAMNWVRQASGKGLEWVGRIRSKYNNYATYYADSVKD |
| | | RFTISRDDSKNTAYLQMNSLKTEDTAVYYCTRHGNFGNSYVSWFAYWG |
| | | QGTLVTVSSGGGGSQVQLKQSGPGLVQPSQSLSITCTVSGFSLTNYGV |
| | | HWVRQSPGKGLEWLGVIWSGGNTDYNTPFTSRLSINKDNSKSQVFFKM |
| | | NSLQSQDTAIYYCARALTYYDYEFAYWGQGTLVTVSAASTKGPSVFPLA |
| | | PSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEVKF |
| | | NWYVDGVEVHNAKTKPCEEQYGSTYRCVSVLTVLHQDWLNGKEYKCK |
| | | VSNKALPAPIEKTISKAKGQPREPQVYTLPPSRKEMTKNQVSLTCLVKGF |
| | | YPSDIAVEWESNGQPENNYKTTPPVLKSDGSFFLYSKLTVDKSRWQQG |
| | | NVFSCSVMHEALHNHYTQKSLSLSPG |
| 39 | Linker | GGGS |
| 40 | Linker | (GGGS)ₙ |
| 41 | Linker | (GSGGS)n |
| 42 | Linker | GGSG |
| 43 | Linker | GGSGG |
| 44 | Linker | GSGSG |
| 45 | Linker | GSGGG |
| 46 | Linker | GGGSG |
| 47 | Linker | GSSSG |
| 48 | Linker | GGGGSGGGGSGGGGSGS |
| 49 | Linker | GGGGSGS |
| 50 | Linker | GGGGSGGGGSGGGGS |
| 51 | Linker | GGGGSGGGGSGGGGSGGGGS |
| 52 | Linker | GGGGS |
| 53 | Linker | GGGGSGGGGS |
| 54 | Linker | GGGS |
| 55 | Linker | GGGSGGGS |
| 56 | Linker | GGGSGGGSGGGS |
| 57 | Linker | GSSGGSGGSGG |
| 58 | Linker | GGGSGGGGSGGGGSGGGGSGGGGS |
| 59 | Linker | GSTSGSGKPGSSEGST |
| 60 | Linker | SKYGPPCPPCPAPEFLG |
| 61 | Linker | GGSLDPKGGGGS |
| 62 | Linker | PKSCDKTHTCPPCPAPELLG |
| 63 | Linker | GKSSGSGSESKS |
| 64 | Linker | GSTSGSGKSSEGKG |
| 65 | Linker | GSTSGSGKSSEGSGSTKG |
| 66 | Linker | GSTSGSGKPGSGEGSTKG |
| 67 | MM1 | MMYCGGNEVLCGPRV |
| 68 | MM1 | GYRWGCEWNCGGITT |
| 69 | MM1 | MMYCGGNEIFCEPRG |
| 70 | MM1 | GYGWGCEWNCGGSSP |
| 71 | MM1 | MMYCGGNEIFCGPRG |
| 72 | MM1 | GYLWGCEWNCGGITT |
| 73 | CM1 | LSGRSDDH |
| | Complex-67 | |
| | Complex-57 | |
| | Cl106 | |
| 74 | CM | ISSGLLSGRSDQH |
| 75 | CM | LSGRSDNH |
| 76 | CM | TSTSGRSANPRG |
| 77 | CM | VHMPLGFLGP |
| 78 | CM | AVGLLAPP |
| 79 | CM | QNQALRMA |
| 80 | CM | ISSGLLSS |
| 81 | CM | ISSGLLSGRSDNH |
| 82 | CM | LSGRSGNH |
| 83 | CM | LSGRSDIH |
| 84 | CM | LSGRSDQH |
| 85 | CM | LSGRSDTH |
| 86 | CM | LSGRSDYH |
| 87 | CM | LSGRSDNP |
| 88 | CM | LSGRSANP |
| 89 | CM | LSGRSANI |
| 90 | CM | LSGRSDNI |
| 91 | CM | ISSGLLSGRSANPRG |
| 92 | CM | AVGLLAPPTSGRSANPRG |
| 93 | CM | AVGLLAPPSGRSANPRG |
| 94 | CM | ISSGLLSGRSDDH |
| 95 | CM | ISSGLLSGRSDIH |
| 96 | CM | ISSGLLSGRSDTH |
| 97 | CM | ISSGLLSGRSDYH |
| 98 | CM | ISSGLLSGRSDNP |
| 99 | CM | ISSGLLSGRSANP |
| 100 | CM | ISSGLLSGRSANI |
| 101 | CM | AVGLLAPPGGLSGRSDDH |
| 102 | CM | AVGLLAPPGGLSGRSDIH |
| 103 | CM | AVGLLAPPGGLSGRSDQH |
| 104 | CM | AVGLLAPPGGLSGRSDTH |
| 105 | CM | AVGLLAPPGGLSGRSDYH |
| 106 | CM | AVGLLAPPGGLSGRSDNP |
| 107 | CM | AVGLLAPPGGLSGRSANP |
| 108 | CM | AVGLLAPPGGLSGRSANI |
| 109 | CM | ISSGLLSGRSDNI |
| 110 | CM | AVGLLAPPGGLSGRSDNI |
| 111 | CM | ISSGLLSGRSGNH |
| 112 | Complex-67 | CAAGGACAATCTGGCTCTGTGTCCACCACCTGTTGGTGGGACCCTCC |
| | Polynucleotide | ATGCACACCTAATACCGGCAGCTCTGGTGGCTCTGGCGGAAGCGGA |
| | Encoding a | GGACTGTCTGGCAGATCCGATGATCACGGCGGAGGATCTGAGGTGC |
| | First | AGCTGGTTGAATCTGGTGGCGGACTGGTTCAGCCTGGCGGATCTCT |
| | Polypeptide | GAAACTGAGCTGTGCCGCCAGCGGCTTCACCTTCAACAAATACGCCA |
| | | TGAACTGGGTCCGACAGGCCCCTGGCAAAGGCCTTGAATGGGTCGC |
| | | CAGAATCAGAAGCAAGTACAACAACTATGCCACCTACTACGCCGACA |
| | | GCGTGAAGGACAGATTCACCATCAGCCGGGACGACAGCAAGAACAC |
| | | CGCCTACCTGCAGATGAACAACCTGAAAACCGAGGACACCGCCGTG |
| | | TACTACTGTGTGCGGCACGGCAACTTCGGCAACAGCTACATCAGCTA |
| | | CTGGGCCTATTGGGGCCAGGGCACACTGGTCACAGTTTCTAGTGGC |
| | | GGAGGCGGATCTGGCGGCGGTGGAAGTGGCGGCGGAGGTTCTCAA |
| | | ACAGTGGTCACCCAAGAGCCTAGCCTGACCGTTTCTCCTGGCGGAA |
| | | CCGTGACACTGACATGCGGATCTTCTACAGGCGCCGTGACCAGCGG |
| | | CAACTACCCTAATTGGGTGCAGCAGAAGCCAGGCCAGGCTCCTAGA |
| | | GGACTGATCGGCGGCACAAAGTTTCTGGCTCCCGGAACACCAGCCA |
| | | GATTCAGCGGTTCTCTGCTCGGAGGAAAGGCCGCTCTGACACTTTCT |
| | | GGCGTGCAGCCTGAGGATGAGGCCGAGTACTATTGCGTGCTGTGGT |
| | | ACAGCAACAGATGGGTGTTCGGCGGAGGCACCAAGCTGACAGTTCT |
| | | TGGAGGTGGCGGTAGCCAGGTCCAGCTGAAACAATCTGGACCCGGA |
| | | CTCGTGCAGCCAAGCCAGAGCCTGTCTATCACCTGTACCGTGTCCG |
| | | GCTTCAGCCTGACCAATTACGGCGTGCACTGGGTTCGACAATCTCCC |
| | | GGCAAGGGACTCGAATGGCTGGGAGTGATTTGGAGCGGCGGCAACA |
| | | CCGACTACAACACCCCATTCACCAGCAGACTGAGCATCAACAAGGAC |
| | | AACAGCAAGTCCCAGGTGTTCTTCAAGATGAACTCCCTGCAGAGCCA |
| | | GGATACCGCCATCTATTACTGCGCTCGGGCCCTGACCTACTATGACT |
| | | ACGAGTTTGCCTACTGGGGACAGGGAACCCTCGTGACAGTGTCTGC |
| | | TGCTAGCACAAAGGGCCCTAGCGTTTTCCCACTGGCTCCCAGCAGC |
| | | AAGTCTACATCCGGTGGAACAGCCGCTCTGGGCTGCCTGGTCAAGG |
| | | ATTACTTTCCCGAGCCAGTGACCGTGTCCTGGAATAGCGGAGCACTG |
| | | ACATCTGGCGTGCACACATTTCCAGCCGTGCTGCAGTCTAGCGGCCT |
| | | GTACTCTCTGTCCAGCGTTGTGACAGTGCCCAGCAGCTCTCTGGGCA |
| | | CCCAGACCTACATCTGCAATGTGAACCACAAGCCTAGCAACACCAAG |
| | | GTGGACAAGAAGGTGGAACCCAAGAGCTGCGATAAGACACACACCT |
| | | GTCCTCCATGTCCTGCTCCAGAGCTGCTCGGAGGCCCTTCCGTGTTT |
| | | CTGTTCCCTCCAAAGCCTAAGGACACCCTGATGATCAGCAGAACCCC |
| | | TGAAGTGACCTGCGTGGTGGTGGATGTGTCCCACGAGGATCCCGAA |
| | | GTGAAGTTCAATTGGTACGTCGACGGCGTGGAAGTGCACAATGCCAA |
| | | GACCAAGCCTTGCGAGGAACAGTACGGCAGCACCTACAGATGCGTG |
| | | TCCGTGCTGACAGTGCTGCACCAGGATTGGCTGAACGGCAAAGAGT |
| | | ACAAGTGCAAGGTGTCCAACAAGGCCCTGCCTGCTCCTATCGAGAAA |
| | | ACCATCAGCAAGGCCAAGGGCCAGCCTAGAGAACCCCAGGTGTACA |
| | | CACTGCCTCCAAGCCGGAAAGAGATGACCAAGAATCAGGTGTCCCT |
| | | GACCTGCCTGGTCAAGGGCTTCTACCCTTCCGATATCGCCGTGGAAT |
| | | GGGAGAGCAATGGACAGCCCGAGAACAACTACAAGACAACCCCTCC |
| | | TGTGCTGAAGTCCGACGGCTCATTCTTCCTGTACAGCAAGCTGACCG |
| | | TGGACAAGAGCAGATGGCAGCAGGGCAACGTGTTCAGCTGCAGCGT |
| | | GATGCACGAGGCCCTGCACAACCACTACACCCAGAAGTCCCTGTCT |
| | | CTGAGCCCCGGCAAA |
| 113 | Polynucleotide | CAAGGCCAGTCTGGCCAAGGTCTTAGTTGTGAAGGTTGGGCGATGA |
| | Encoding a | ATAGAGAACAATGTCGAGCCGGAGGTGGCTCGAGCGGCGGCTCTAT |
| | Second | CTCTTCCGGACTGCTGTCCGGCAGATCCGACCAGCACGGCGGAGGA |
| | Polypeptide | TCCCAAATCCTGCTGACACAGTCTCCTGTCATACTGAGTGTCTCCCC |
| | Complex-57 | CGGCGAGAGAGTCTCTTTCTCATGTCGGGCCAGTCAGTCTATTGGGA |
| | Complex-67 | CTAACATACACTGGTACCAGCAACGCACCAACGGAAGCCCGCGCCT |
| | Cl106 | GCTGATTAAATATGCGAGCGAAAGCATTAGCGGCATTCCGAGCCGCT |
| | | TTAGCGGCAGCGGCAGCGGCACCGATTTTACCCTGAGCATTAACAG |
| | | CGTGGAAAGCGAAGATATTGCGGATTATTATTGCCAGCAGAACAACA |
| | | ACTGGCCGACCACCTTTGGCGCGGGCACCAAACTGGAACTGAAACG |
| | | TACGGTGGCTGCACCATCTGTCTTCATCTTCCCGCCATCTGATGAGC |
| | | AGTTGAAATCTGGAACTGCCTCTGTTGTGTGCCTGCTGAATAACTTCT |
| | | ATCCCAGAGAGGCCAAAGTACAGTGGAAGGTGGATAACGCCCTCCA |
| | | ATCGGGTAACTCCCAGGAGAGTGTCACAGAGCAGGACAGCAAGGAC |
| | | AGCACCTACAGCCTCAGCAGCACCCTGACGCTGAGCAAAGCAGACT |
| | | ACGAGAAACACAAAGTCTACGCCTGCGAAGTCACCCATCAGGGCCT |
| | | GAGCTCGCCCGTCACAAAGAGCTTCAACAGGGGAGAGTGT |
| 114 | Polynucleotide | GATAAGACCCACACCTGTCCTCCATGTCCTGCTCCAGAACTGCTCGG |
| | Encoding a | CGGACCTTCCGTGTTCCTGTTTCCTCCAAAGCCTAAGGACACCCTGA |
| | Third | TGATCAGCAGAACCCCTGAAGTGACCTGCGTGGTGGTGGATGTGTC |
| | Polypeptide | CCACGAGGATCCCGAAGTGAAGTTCAATTGGTACGTGGACGGCGTG |
| | Complex-57 | GAAGTGCACAACGCCAAGACAAAGCCCTGCGAGGAACAGTACGGCA |
| | Complex-67 | GCACCTACAGATGCGTGTCCGTGCTGACAGTGCTGCACCAGGATTG |
| | | GCTGAACGGCAAAGAGTACAAGTGCAAGGTGTCCAACAAGGCCCTG |
| | | CCTGCTCCTATCGAGAAAACCATCAGCAAGGCCAAGGGCCAGCCTA |
| | | GAGAACCCCAGGTGTACACACTGCCTCCAAGCCGGGAAGAGATGAC |
| | | CAAGAACCAGGTGTCCCTGACCTGCCTGGTCAAGGGCTTCTACCCTT |
| | | CCGATATCGCCGTGGAATGGGAGAGCAATGGACAGCCCGAGAACAA |
| | | CTACGACACCACACCTCCAGTGCTGGACAGCGACGGCTCATTCTTCC |
| | | TGTACAGCGACCTGACCGTGGACAAGAGCAGATGGCAGCAGGGCAA |
| | | CGTGTTCAGCTGCAGCGTGATGCACGAGGCCCTGCACAACCACTAC |
| | | ACCCAGAAGTCCCTGAGCCTGTCTCCTGGCAAA |
| 115 | Light | CAAGGACAATCTGGACAGGGCCTGAGCTGTGAAGGCTGGGCCATGA |
| | Chain/Second | ATAGAGAGCAGTGCAGAGCTGGCGGCGGATCTTCTGGCGGCTCTAT |
| | Polypeptide | CTCTTCTGGACTGCTGAGCGGCAGAAGCGATCAACACGGCGGAGGC |
| | Complex-57 | TCTCAGATCCTGCTGACACAGAGCCCCGTGATCCTGTCTGTGTCTCC |
| | Complex-67 | TGGCGAGAGAGTGTCCTTCAGCTGTAGAGCCAGCCAGTCCATCGGC |
| | Cl106 | ACCAACATCCACTGGTATCAGCAGCGGACCAACGGCAGCCCCAGAC |
| | | TGCTGATTAAGTACGCCAGCGAGAGCATCAGCGGCATCCCCAGCAG |
| | | ATTTTCTGGCAGCGGCTCTGGCACCGACTTCACCCTGAGCATCAACA |
| | | GCGTGGAAAGCGAGGATATCGCCGACTACTACTGCCAGCAGAACAA |
| | | CAACTGGCCCACCACCTTTGGAGCCGGCACCAAGCTGGAACTGAAG |
| | | AGAACAGTGGCCGCTCCTAGCGTGTTCATCTTCCCACCTTCCGACGA |
| | | GCAGCTGAAAAGCGGCACAGCCTCTGTCGTGTGCCTGCTGAACAAC |
| | | TTCTACCCCAGAGAAGCCAAGGTGCAGTGGAAGGTGGACAACGCCC |
| | | TGCAGAGCGGCAATAGCCAAGAGTCTGTGACCGAGCAGGACAGCAA |
| | | GGACTCCACCTACAGCCTGAGCAGCACCCTGACACTGAGCAAGGCC |
| | | GACTACGAGAAGCACAAAGTGTACGCCTGCGAAGTGACCCACCAGG |
| | | GCCTTTCTAGCCCTGTGACCAAGAGCTTCAACCGGGGCGAGTGT |
| 116 | spacer | QGQSGS |
| 117 | spacer | QGQSGQG |
| 118 | spacer | QGQSGS |
| 119 | spacer | QGQSGQG |
| 120 | First | VSTTCWWDPPCTPNTGSSGGSGGSGGLSGRSDDHGGGSEVQLVESG |
| | polypeptide | GGLVQPGGSLKLSCAASGFTFNKYAMNWVRQAPGKGLEWVARIRSKY |
| | without spacer | NNYATYYADSVKDRFTISRDDSKNTAYLQMNNLKTEDTAVYYCVRHGNF |
| | and with a | GNSYISYWAYWGQGTLVTVSS GGGGSGGGGSGGGGSQTVVTQEPSL |
| | terminal lysine | TVSPGGTVTLTCGSSTGAVTSGNYPNWVQQKPGQAPRGLIGGTKFLAP |
| | Complex-67 | GTPARFSGSLLGGKAALTLSGVQPEDEAEYYCVLWYSNRWVFGGGTKL |
| | | TVLGGGGSQVQLKQSGPGLVQPSQSLSITCTVSGFSLTNYGVHWVRQS |
| | | PGKGLEWLGVIWSGGNTDYNTPFTSRLSINKDNSKSQVFFKMNSLQSQ |
| | | DTAIYYCARALTYYDYEFAYWGQGTLVTVSAASTKGPSVFPLAPSSKST |
| | | SGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPE |
| | | LLGGPSVFLFPPKPKDTLMISRTPEVTCWVDVSHEDPEVKFNWYVDGV |
| | | EVHNAKTKPCEEQYGSTYRCVSVLTVLHQDWLNGKEYKCKVSNKALPA |
| | | PIEKTISKAKGQPREPQVYTLPPSRKEMTKNQVSLTCLVKGFYPSDIAVE |
| | | WESNGQPENNYKTTPPVLKSDGSFFLYSKLTVDKSRWQQGNVFSCSV |
| | | MHEALHNHYTQKSLSLSPGK |
| 121 | BLANK | |
| 122 | Anti-CD3 scFv | QTVVTQEPSFSVSPGGTVTLTCRSSTGAVTTSNYANWVQQTPGQAPR |
| | V16 | GLIGGTNKRAPGVPDRFSGSILGNKAALTITGAQADDESDYYCALWYSN |
| | Complex-57 | LWVFGGGTKLTVLGGGGSGGGGSGGGGSEVQLVESGGGLVQPGGSL |
| | Cl106 | KLSCAASGFTFSTYAMNWVRQASGKGLEWVGRIRSKYNNYATYYADSV |
| | | KDRFTISRDDSKNTAYLQMNSLKTEDTAVYYCTRHGNFGNSYVSWFAY |
| | | WGQGTLVTVSS |
| 123 | Cl106 - Heavy | QGQSGSGYLWGCEWNCGGITTGSSGGSGGSGGLSGRSDDHGGGSQ |
| | Chain CRF41- | TVVTQEPSFSVSPGGTVTLTCRSSTGAVTTSNYANWVQQTPGQAPRGL |
| | 2008- | IGGTNKRAPGVPDRFSGSILGNKAALTITGAQADDESDYYCALWYSNLW |
| | C225v5Fcmt4- | VFGGGTKLTVLGGGGSGGGGSGGGGSEVQLVESGGGLVQPGGSLKLS |
| | h20GG-0011- | CAASGFTFSTYAMNWVRQASGKGLEWVGRIRSKYNNYATYYADSVKD |
| | v16sc-H-N | RFTISRDDSKNTAYLQMNSLKTEDTAVYYCTRHGNFGNSYVSWFAYWG |
| | | QGTLVTVSSGGGGSQVQLKQSGPGLVQPSQSLSITCTVSGFSLTNYGV |
| | | HWVRQSPGKGLEWLGVIWSGGNTDYNTPFTSRLSINKDNSKSQVFFKM |
| | | NSLQSQDTAIYYCARALTYYDYEFAYWGQGTLVTVSAASTKGPSVFPLA |
| | | PSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTC |
| | | PPCPAPEFEGGPSVFLFPPKPKDTLMISRTPEVTCVVVDVSHEDPEVKF |
| | | NWYVDGVEVHNAKTKPREEQYQSTYRVVSVLTVLHQDWLNGKEYKCK |
| | | VSNKALPASIEKTISKAKGQPREPQVYTLPPSREEMTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| 124 | Fc Cl106 | PAPEFEGGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNK |
| | | ALPASIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| 125 | Cl106 Heavy | CAAGGCCAGTCTGGATCCGGTTATCTGTGGGGTTGCGAGTGGAATT |
| | Chain - CRF41- | GCGGAGGGATCACTACAGGCTCGAGCGGTGGCAGCGGTGGCTCTG |
| | 2008- | GTGGTCTGAGCGGCCGTTCCGATGATCATGGCGGCGGTTCTCAAAC |
| | C225v5Fcmt4- | TGTAGTAACTCAAGAACCAAGCTTCTCCGTCTCCCCTGGGGGAACAG |
| | h20GG-0011- | TCACACTTACCTGCCGAAGTAGTACAGGTGCTGTTACGACCAGTAAC |
| | v16sc-H-N | TATGCCAATTGGGTACAACAAACGCCTGGTCAGGCTCCGCGCGGATT |
| | | GATAGGAGGCACGAATAAACGGGCACCCGGTGTCCCGGACAGATTC |
| | | AGCGGAAGCATACTCGGTAATAAGGCAGCTCTTACTATCACTGGGGC |
| | | CCAAGCTGATGATGAAAGTGATTATTATTGTGCGCTCTGGTACAGCA |
| | | ACCTCTGGGTGTTTGGGGGTGGCACGAAACTTACTGTCTTGGGCGG |
| | | CGGCGGATCAGGGGGAGGTGGCTCTGGAGGAGGAGGCTCAGAAGT |
| | | CCAACTGGTCGAATCCGGGGGAGGGCTCGTACAGCCGGGTGGGTC |
| | | CCTCAAACTCTCTTGTGCGGCCTCAGGGTTTACCTTCAGTACATACG |
| | | CGATGAATTGGGTCCGGCAGGCCAGTGGGAAAGGGCTCGAATGGGT |
| | | AGGACGAATCCGATCAAAATACAACAACTACGCTACTTATTACGCTGA |
| | | TTCCGTGAAGGACAGATTCACAATATCCCGCGACGATAGCAAGAATA |
| | | CGGCATATCTTCAGATGAATTCTCTTAAAACTGAGGATACCGCTGTGT |
| | | ATTACTGCACAAGACATGGTAATTTTGGAAACTCATATGTCTCTTGGT |
| | | TCGCTTATTGGGGACAGGGCACGTTGGTTACCGTGTCTAGCGGAGG |
| | | TGGTGGATCCCAGGTGCAGCTGAAACAGAGCGGCCCGGGCCTGGT |
| | | GCAGCCGAGCCAGAGCCTGAGCATTACCTGCACCGTGAGCGGCTTT |
| | | AGCCTGACCAACTATGGCGTGCATTGGGTGCGCCAGAGCCCGGGCA |
| | | AAGGCCTGGAATGGCTGGGCGTGATTTGGAGCGGCGGCAACACCGA |
| | | TTATAACACCCCGTTTACCAGCCGCCTGAGCATTAACAAAGATAACA |
| | | GCAAAAGCCAGGTGTTTTTTAAAATGAACAGCCTGCAAAGCCAGGAT |
| | | ACCGCGATTTATTATTGCGCGCGCGCGCTGACCTATTATGATTATGA |
| | | ATTTGCGTATTGGGGCCAGGGCACCCTGGTGACCGTGAGCGCGGCT |
| | | AGCACCAAGGGCCCATCGGTCTTCCCCCTGGCACCCTCCTCCAAGA |
| | | GCACCTCTGGGGGCACAGCGGCCCTGGGCTGCCTGGTCAAGGACT |
| | | ACTTCCCCGAACCGGTGACGGTGTCGTGGAACTCAGGCGCCCTGAC |
| | | CAGCGGCGTGCACACCTTCCCGGCTGTCCTACAGTCCTCAGGACTC |
| | | TACTCCCTCAGCAGCGTGGTGACCGTGCCCTCCAGCAGCTTGGGCA |
| | | CCCAGACCTACATCTGCAACGTGAATCACAAGCCCAGCAACACCAAG |
| | | GTGGACAAGAAAGTTGAGCCCAAATCTTGTGACAAAACTCACACATG |
| | | CCCACCGTGCCCAGCACCTGAATTTGAAGGGGGACCGTCAGTCTTC |
| | | CTCTTCCCCCCAAAACCCAAGGACACCCTCATGATCTCCCGGACCCC |
| | | TGAGGTCACATGCGTGGTGGTGGACGTGAGCCACGAAGACCCTGAG |
| | | GTCAAGTTCAACTGGTACGTGGACGGCGTGGAGGTGCATAATGCCA |
| | | AGACAAAGCCGCGGGAGGAGCAGTACCAGAGCACGTACCGTGTGGT |
| | | CAGCGTCCTCACCGTCCTGCACCAGGACTGGCTGAATGGCAAGGAG |
| | | TACAAGTGCAAGGTCTCCAACAAAGCCCTCCCAGCCTCAATCGAGAA |
| | | AACCATCTCCAAAGCCAAAGGGCAGCCCCGAGAACCACAGGTGTAC |
| | | ACCCTGCCCCCATCCCGGGAGGAGATGACCAAGAACCAGGTCAGCC |
| | | TGACCTGCCTGGTCAAAGGCTTCTATCCCAGCGACATCGCCGTGGA |
| | | GTGGGAGAGCAATGGGCAGCCGGAGAACAACTACAAGACCACGCCT |
| | | CCCGTGCTGGACTCCGACGGCTCCTTCTTCCTCTACAGCAAGCTCAC |
| | | CGTGGACAAGAGCAGGTGGCAGCAGGGGAACGTCTTCTCATGCTCC |
| | | GTGATGCATGAGGCTCTGCACAACCACTACACGCAGAAGAGCCTCTC |
| | | CCTGTCTCCGGGTAAA |
| 126 | Linker | (GGGGS)n |
| 127 | Linker | GGGSSGGS |
| 128 | VH1 CDR1 | TYAMN |
| | Complex-57 | |
| | Cl106 (control) | |
| 129 | VH1 CDR2 | RIRSKYNNYATYYADSVKD |
| | Complex-57 | |
| | Cl106 (control) | |
| 130 | VH1 CDR3 | HGNFGNSYVSWFAY |
| | Complex-57 | |
| | Cl106 (control) | |
| 131 | VL1 CDR1 | RSSTGAVTTSNYAN |
| | Complex-57 | |
| | Cl106 (control) | |
| 132 | VL1 CDR2 | GTNKRAP |
| | Complex-57 | |
| | Cl106 (control) | |
| 133 | VL1 CDR3 | ALWYSNLWV |
| | Complex-57 | |
| | Cl106 (control) | |
| 134 | VH1 Domain | EVQLVESGGGLVQPGGSLKLSCAASGFTFSTYAMNWVRQASGKGLEW |
| | Complex-57 | VGRIRSKYNNYATYYADSVKDRFTISRDDSKNTAYLQMNSLKTEDTAVY |
| | Cl106 (control) | YCTRHGNFGNSYVSWFAYWGQGTLVTVSS |
| 135 | VL1 Domain | QTVVTQEPSFSVSPGGTVTLTCRSSTGAVTTSNYANWVQQTPGQAPR |
| | Complex-57 | GLIGGTNKRAPGVPDRFSGSILGNKAALTITGAQADDESDYYCALWYSN |
| | Cl106 (control) | LWVFGGGTKLTVL |
| 136 | Complex-57 | QGQSGSGYLWGCEWNCGGITTGSSGGSGGSGGLSGRSDDHGGGSQ |
| | First | TVVTQEPSFSVSPGGTVTLTCRSSTGAVTTSNYANWVQQTPGQAPRGL |
| | Polypeptide | IGGTNKRAPGVPDRFSGSILGNKAALTITGAQADDESDYYCALWYSNLW |
| | with terminal | VFGGGTKLTVLGGGGSGGGGSGGGGSEVQLVESGGGLVQPGGSLKLS |
| | lysine | CAASGFTFSTYAMNWVRQASGKGLEWVGRIRSKYNNYATYYADSVKD |
| | | RFTISRDDSKNTAYLQMNSLKTEDTAVYYCTRHGNFGNSYVSWFAYWG |
| | | QGTLVTVSSGGGGSQVQLKQSGPGLVQPSQSLSITCTVSGFSLTNYGV |
| | | HWVRQSPGKGLEWLGVIWSGGNTDYNTPFTSRLSINKDNSKSQVFFKM |
| | | NSLQSQDTAIYYCARALTYYDYEFAYWGQGTLVTVSAASTKGPSVFPLA |
| | | PSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEVKF |
| | | NWYVDGVEVHNAKTKPCEEQYGSTYRCVSVLTVLHQDWLNGKEYKCK |
| | | VSNKALPAPIEKTISKAKGQPREPQVYTLPPSRKEMTKNQVSLTCLVKGF |
| | | YPSDIAVEWESNGQPENNYKTTPPVLKSDGSFFLYSKLTVDKSRWQQG |
| | | NVFSCSVMHEALHNHYTQKSLSLSPGK |
| 137 | First | [QGQSGS]VSTTCWWDPPCTPNTGSSGGSGGSGGLSGRSDDHGGGSE |
| | Polypeptide | VQLVESGGGLVQPGGSLKLSCAASGFTFNKYAMNWVRQAPGKGLEWV |
| | Complex-67 | ARIRSKYNNYATYYADSVKDRFTISRDDSKNTAYLQMNNLKTEDTAVYY |
| | w/terminal | CVRHGNFGNSYISYWAYWGQGTLVTVSS [symbol] QTV |
| | lysine | VTQEPSLTVSPGGTVTLTCGSSTGAVTSGNYPNWVQQKPGQAPRGLIG |
| | | GTKFLAPGTPARFSGSLLGGKAALTLSGVQPEDEAEYYCVLWYSNRWV |
| | | FGGGTKLTVLGGGGSQVQLKQSGPGLVQPSQSLSITCTVSGFSLTNYG |
| | | VHWVRQSPGKGLEWLGVIWSGGNTDYNTPFTSRLSINKDNSKSQVFFK |
| | | MNSLQSQDTAIYYCARALTYYDYEFAYWGQGTLVTVSAASTKGPSVFPL |
| | | APSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHT |
| | | CPPCPAPELLGGPSVFLFPPKPKDTLMISRTPEVTCVVVDVSHEDPEVK |
| | | FNWYVDGVEVHNAKTKPCEEQYGSTYRCVSVLTVLHQDWLNGKEYKC |
| | | KVSNKALPAPIEKTISKAKGQPREPQVYTLPPSRKEMTKNQVSLTCLVKG |
| | | FYPSDIAVEWESNGQPENNYKTTPPVLKSDGSFFLYSKLTVDKSRWQQ |
| | | GNVFSCSVMHEALHNHYTQKSLSLSPGK |
| 138 | BLANK | |
| 139 | Polynucleotide | CAAGGACAATCTGGCTCTGTGTCCACCACCTGTTGGTGGGACCCTCC |
| | encoding 1^{st} | ATGCACACCTAATACCGGCAGCTCTGGTGGCTCTGGCGGAAGCGGA |
| | polypeptide w/o | GGACTGTCTGGCAGATCCGATGATCACGGCGGAGGATCTGAGGTGC |
| | terminal lysine | AGCTGGTTGAATCTGGTGGCGGACTGGTTCAGCCTGGCGGATCTCT |
| | Complex-67 | GAAACTGAGCTGTGCCGCCAGCGGCTTCACCTTCAACAAATACGCCA |
| | | TGAACTGGGTCCGACAGGCCCCTGGCAAAGGCCTTGAATGGGTCGC |
| | | CAGAATCAGAAGCAAGTACAACAACTATGCCACCTACTACGCCGACA |
| | | GCGTGAAGGACAGATTCACCATCAGCCGGGACGACAGCAAGAACAC |
| | | CGCCTACCTGCAGATGAACAACCTGAAAACCGAGGACACCGCCGTG |
| | | TACTACTGTGTGCGGCACGGCAACTTCGGCAACAGCTACATCAGCTA |
| | | CTGGGCCTATTGGGGCCAGGGCACACTGGTCACAGTTTCTAGTGGC |
| | | GGAGGCGGATCTGGCGGCGGTGGAAGTGGCGGCGGAGGTTCTCAA |
| | | ACAGTGGTCACCCAAGAGCCTAGCCTGACCGTTTCTCCTGGCGGAA |
| | | CCGTGACACTGACATGCGGATCTTCTACAGGCGCCGTGACCAGCGG |
| | | CAACTACCCTAATTGGGTGCAGCAGAAGCCAGGCCAGGCTCCTAGA |
| | | GGACTGATCGGCGGCACAAAGTTTCTGGCTCCCGGAACACCAGCCA |
| | | GATTCAGCGGTTCTCTGCTCGGAGGAAAGGCCGCTCTGACACTTTCT |
| | | GGCGTGCAGCCTGAGGATGAGGCCGAGTACTATTGCGTGCTGTGGT |
| | | ACAGCAACAGATGGGTGTTCGGCGGAGGCACCAAGCTGACAGTTCT |
| | | TGGAGGTGGCGGTAGCCAGGTCCAGCTGAAACAATCTGGACCCGGA |
| | | CTCGTGCAGCCAAGCCAGAGCCTGTCTATCACCTGTACCGTGTCCG |
| | | GCTTCAGCCTGACCAATTACGGCGTGCACTGGGTTCGACAATCTCCC |
| | | GGCAAGGGACTCGAATGGCTGGGAGTGATTTGGAGCGGCGGCAACA |
| | | CCGACTACAACACCCCATTCACCAGCAGACTGAGCATCAACAAGGAC |
| | | AACAGCAAGTCCCAGGTGTTCTTCAAGATGAACTCCCTGCAGAGCCA |
| | | GGATACCGCCATCTATTACTGCGCTCGGGCCCTGACCTACTATGACT |
| | | ACGAGTTTGCCTACTGGGGACAGGGAACCCTCGTGACAGTGTCTGC |
| | | TGCTAGCACAAAGGGCCCTAGCGTTTTCCCACTGGCTCCCAGCAGC |
| | | AAGTCTACATCCGGTGGAACAGCCGCTCTGGGCTGCCTGGTCAAGG |
| | | ATTACTTTCCCGAGCCAGTGACCGTGTCCTGGAATAGCGGAGCACTG |
| | | ACATCTGGCGTGCACACATTTCCAGCCGTGCTGCAGTCTAGCGGCCT |
| | | GTACTCTCTGTCCAGCGTTGTGACAGTGCCCAGCAGCTCTCTGGGCA |
| | | CCCAGACCTACATCTGCAATGTGAACCACAAGCCTAGCAACACCAAG |
| | | GTGGACAAGAAGGTGGAACCCAAGAGCTGCGATAAGACACACACCT |
| | | GTCCTCCATGTCCTGCTCCAGAGCTGCTCGGAGGCCCTTCCGTGTTT |
| | | CTGTTCCCTCCAAAGCCTAAGGACACCCTGATGATCAGCAGAACCCC |
| | | TGAAGTGACCTGCGTGGTGGTGGATGTGTCCCACGAGGATCCCGAA |
| | | GTGAAGTTCAATTGGTACGTCGACGGCGTGGAAGTGCACAATGCCAA |
| | | GACCAAGCCTTGCGAGGAACAGTACGGCAGCACCTACAGATGCGTG |
| | | TCCGTGCTGACAGTGCTGCACCAGGATTGGCTGAACGGCAAAGAGT |
| | | ACAAGTGCAAGGTGTCCAACAAGGCCCTGCCTGCTCCTATCGAGAAA |
| | | ACCATCAGCAAGGCCAAGGGCCAGCCTAGAGAACCCCAGGTGTACA |
| | | CACTGCCTCCAAGCCGGAAAGAGATGACCAAGAATCAGGTGTCCCT |
| | | GACCTGCCTGGTCAAGGGCTTCTACCCTTCCGATATCGCCGTGGAAT |
| | | GGGAGAGCAATGGACAGCCCGAGAACAACTACAAGACAACCCCTCC |
| | | TGTGCTGAAGTCCGACGGCTCATTCTTCCTGTACAGCAAGCTGACCG |
| | | TGGACAAGAGCAGATGGCAGCAGGGCAACGTGTTCAGCTGCAGCGT |
| | | GATGCACGAGGCCCTGCACAACCACTACACCCAGAAGTCCCTGTCT |
| | | CTGAGCCCCGGC |
| 140 | 3^{rd} Polypeptide | DKTHTCPPCPAPELLGGPSVFLFPPKPKDTLMISRTPEVTCVVVDVSHE |
| | w/C-terminal | DPEVKFNWYVDGVEVHNAKTKPCEEQYGSTYRCVSVLTVLHQDWLNG |
| | lysine | KEYKCKVSNKALPAPIEKTISKAKGQPREPQVYTLPPSREEMTKNQVSLT |
| | Complex-67 | CLVKGFYPSDIAVEWESNGQPENNYDTTPPVLDSDGSFFLYSDLTVDKS |
| | Complex-57 | RWQQGNVFSCSVMHEALHNHYTQKSLSLSPGK |
| 141 | Polynucleotide | GATAAGACCCACACCTGTCCTCCATGTCCTGCTCCAGAACTGCTCGG |
| | encoding 3^{rd} | CGGACCTTCCGTGTTCCTGTTTCCTCCAAAGCCTAAGGACACCCTGA |
| | Polypeptide | TGATCAGCAGAACCCCTGAAGTGACCTGCGTGGTGGTGGATGTGTC |
| | without codon | CCACGAGGATCCCGAAGTGAAGTTCAATTGGTACGTGGACGGCGTG |
| | encoding C- | GAAGTGCACAACGCCAAGACAAAGCCCTGCGAGGAACAGTACGGCA |
| | terminal lysine | GCACCTACAGATGCGTGTCCGTGCTGACAGTGCTGCACCAGGATTG |
| | Complex-67 | GCTGAACGGCAAAGAGTACAAGTGCAAGGTGTCCAACAAGGCCCTG |
| | Complex-57 | CCTGCTCCTATCGAGAAAACCATCAGCAAGGCCAAGGGCCAGCCTA |
| | | GAGAACCCCAGGTGTACACACTGCCTCCAAGCCGGGAAGAGATGAC |
| | | CAAGAACCAGGTGTCCCTGACCTGCCTGGTCAAGGGCTTCTACCCTT |
| | | CCGATATCGCCGTGGAATGGGAGAGCAATGGACAGCCCGAGAACAA |
| | | CTACGACACCACACCTCCAGTGCTGGACAGCGACGGCTCATTCTTCC |
| | | TGTACAGCGACCTGACCGTGGACAAGAGCAGATGGCAGCAGGGCAA |
| | | CGTGTTCAGCTGCAGCGTGATGCACGAGGCCCTGCACAACCACTAC |
| | | ACCCAGAAGTCCCTGAGCCTGTCTCCTGGC |
| 142 | Polynucleotide | CAAGGACAATCTGGATCCGGCTATCTGTGGGGCTGCGAGTGGAATT |
| | encoding 1^{st} | GTGGCGGCATCACAACAGGCTCTAGCGGCGGAAGCGGAGGATCTG |
| | polypeptide | GTGGACTGTCTGGCAGATCCGATGATCATGGCGGCGGATCCCAGAC |
| | (without codon | CGTGGTCACACAAGAGCCTAGCTTCTCCGTGTCTCCTGGCGGCACA |
| | for C-terminal | GTGACCCTGACATGCAGATCTTCTACAGGCGCCGTGACCACCAGCA |
| | lysine) | ACTACGCCAATTGGGTGCAGCAGACCCCTGGACAGGCTCCTAGAGG |
| | Complex-57 | ACTGATCGGCGGCACCAACAAAAGAGCCCCTGGCGTCCCAGATAGA |
| | | TTCAGCGGCTCTATCCTGGGCAACAAGGCCGCACTGACAATCACAG |
| | | GCGCCCAGGCCGATGACGAGAGCGATTACTATTGCGCCCTGTGGTA |
| | | CAGCAACCTGTGGGTTTTCGGCGGAGGCACCAAGCTGACAGTTCTT |
| | | GGCGGAGGCGGAAGTGGTGGTGGCGGATCTGGTGGCGGTGGATCT |
| | | GAAGTGCAGCTGGTGGAATCTGGCGGAGGACTTGTTCAGCCAGGCG |
| | | GCTCTCTGAAGCTGTCTTGTGCCGCCTCCGGCTTCACCTTTAGCACC |
| | | TACGCCATGAACTGGGTCCGACAGGCCTCTGGCAAAGGCCTGGAAT |
| | | GGGTCGGACGGATCAGAAGCAAGTACAACAATTACGCCACCTACTAC |
| | | GCCGACAGCGTGAAGGACAGATTCACCATCAGCCGGGACGACAGCA |
| | | AGAACACCGCCTACCTGCAGATGAACAGCCTGAAAACCGAGGACAC |
| | | CGCCGTGTACTACTGCACCAGACACGGCAACTTCGGCAACAGCTAT |
| | | GTGTCTTGGTTTGCCTACTGGGGCCAGGGCACACTGGTCACAGTTA |
| | | GTTCTGGCGGCGGAGGTTCTCAGGTGCAGCTGAAACAGTCTGGCCC |
| | | TGGACTGGTGCAGCCTAGCCAGTCTCTGAGCATCACCTGTACCGTGT |
| | | CCGGCTTCTCCCTGACCAATTACGGCGTGCACTGGGTTCGACAATCC |
| | | CCAGGCAAGGGACTCGAATGGCTGGGAGTGATTTGGAGCGGCGGC |
| | | AACACCGACTACAACACCCCATTCACCAGCAGACTGTCCATCAACAA |
| | | GGACAACAGCAAGTCCCAGGTGTTCTTCAAGATGAACTCCCTGCAGA |
| | | GCCAGGATACCGCCATCTATTACTGCGCTCGGGCCCTGACCTACTAT |
| | | GACTACGAGTTCGCCTATTGGGGACAGGGAACCCTCGTGACAGTGT |
| | | CTGCCGCTAGCACAAAGGGCCCTAGCGTTTTCCCACTGGCTCCCAG |
| | | CAGCAAGTCTACATCCGGTGGAACAGCCGCTCTGGGCTGCCTGGTC |
| | | AAGGATTACTTTCCCGAGCCAGTGACCGTGTCCTGGAATAGCGGAG |
| | | CACTGACATCTGGCGTGCACACATTTCCAGCCGTGCTGCAGTCTAGC |
| | | GGCCTGTACTCTCTGTCCAGCGTTGTGACAGTGCCCAGCAGCTCTCT |
| | | GGGCACCCAGACCTACATCTGCAATGTGAACCACAAGCCTAGCAACA |
| | | CCAAGGTGGACAAGAAGGTGGAACCCAAGAGCTGCGATAAGACACA |
| | | CACCTGTCCTCCATGTCCTGCTCCAGAGCTGCTCGGAGGCCCTTCC |
| | | GTGTTTCTGTTCCCTCCAAAGCCTAAGGACACCCTGATGATCAGCAG |
| | | AACCCCTGAAGTGACCTGCGTGGTGGTGGATGTGTCCCACGAGGAT |
| | | CCCGAAGTGAAGTTCAATTGGTACGTCGACGGCGTGGAAGTGCACA |
| | | ATGCCAAGACCAAGCCTTGCGAGGAACAGTACGGCAGCACCTACAG |
| | | ATGCGTGTCCGTGCTGACAGTGCTGCACCAGGATTGGCTGAACGGC |
| | | AAAGAGTACAAGTGCAAGGTGTCCAACAAGGCCCTGCCTGCTCCTAT |
| | | CGAGAAAACCATCAGCAAGGCCAAGGGCCAGCCTAGAGAACCCCAG |
| | | GTGTACACACTGCCTCCAAGCCGGAAAGAGATGACCAAGAATCAGGT |
| | | GTCCCTGACCTGCCTGGTCAAGGGCTTCTACCCTTCCGATATCGCCG |
| | | TGGAATGGGAGAGCAATGGACAGCCCGAGAACAACTACAAGACAAC |
| | | CCCTCCTGTGCTGAAGTCCGACGGCTCATTCTTCCTGTACAGCAAGC |
| | | TGACCGTGGACAAGAGCAGATGGCAGCAGGGCAACGTGTTCAGCTG |
| | | CAGCGTGATGCACGAGGCCCTGCACAACCACTACACCCAGAAGTCC |
| | | CTGTCTCTGAGCCCCGGC |
| 143 | Polynucleotide | CAAGGACAATCTGGATCCGGCTATCTGTGGGGCTGCGAGTGGAATT |
| | encoding 1^{st} | GTGGCGGCATCACAACAGGCTCTAGCGGCGGAAGCGGAGGATCTG |
| | polypeptide | GTGGACTGTCTGGCAGATCCGATGATCATGGCGGCGGATCCCAGAC |
| | (with codon for | CGTGGTCACACAAGAGCCTAGCTTCTCCGTGTCTCCTGGCGGCACA |
| | C-terminal | GTGACCCTGACATGCAGATCTTCTACAGGCGCCGTGACCACCAGCA |
| | lysine) | ACTACGCCAATTGGGTGCAGCAGACCCCTGGACAGGCTCCTAGAGG |
| | Complex-57 | ACTGATCGGCGGCACCAACAAAAGAGCCCCTGGCGTCCCAGATAGA |
| | | TTCAGCGGCTCTATCCTGGGCAACAAGGCCGCACTGACAATCACAG |
| | | GCGCCCAGGCCGATGACGAGAGCGATTACTATTGCGCCCTGTGGTA |
| | | CAGCAACCTGTGGGTTTTCGGCGGAGGCACCAAGCTGACAGTTCTT |
| | | GGCGGAGGCGGAAGTGGTGGTGGCGGATCTGGTGGCGGTGGATCT |
| | | GAAGTGCAGCTGGTGGAATCTGGCGGAGGACTTGTTCAGCCAGGCG |
| | | GCTCTCTGAAGCTGTCTTGTGCCGCCTCCGGCTTCACCTTTAGCACC |
| | | TACGCCATGAACTGGGTCCGACAGGCCTCTGGCAAAGGCCTGGAAT |
| | | GGGTCGGACGGATCAGAAGCAAGTACAACAATTACGCCACCTACTAC |
| | | GCCGACAGCGTGAAGGACAGATTCACCATCAGCCGGGACGACAGCA |
| | | AGAACACCGCCTACCTGCAGATGAACAGCCTGAAAACCGAGGACAC |
| | | CGCCGTGTACTACTGCACCAGACACGGCAACTTCGGCAACAGCTAT |
| | | GTGTCTTGGTTTGCCTACTGGGGCCAGGGCACACTGGTCACAGTTA |
| | | GTTCTGGCGGCGGAGGTTCTCAGGTGCAGCTGAAACAGTCTGGCCC |
| | | TGGACTGGTGCAGCCTAGCCAGTCTCTGAGCATCACCTGTACCGTGT |
| | | CCGGCTTCTCCCTGACCAATTACGGCGTGCACTGGGTTCGACAATCC |
| | | CCAGGCAAGGGACTCGAATGGCTGGGAGTGATTTGGAGCGGCGGC |
| | | AACACCGACTACAACACCCCATTCACCAGCAGACTGTCCATCAACAA |
| | | GGACAACAGCAAGTCCCAGGTGTTCTTCAAGATGAACTCCCTGCAGA |
| | | GCCAGGATACCGCCATCTATTACTGCGCTCGGGCCCTGACCTACTAT |
| | | GACTACGAGTTCGCCTATTGGGGACAGGGAACCCTCGTGACAGTGT |
| | | CTGCCGCTAGCACAAAGGGCCCTAGCGTTTTCCCACTGGCTCCCAG |
| | | CAGCAAGTCTACATCCGGTGGAACAGCCGCTCTGGGCTGCCTGGTC |
| | | AAGGATTACTTTCCCGAGCCAGTGACCGTGTCCTGGAATAGCGGAG |
| | | CACTGACATCTGGCGTGCACACATTTCCAGCCGTGCTGCAGTCTAGC |
| | | GGCCTGTACTCTCTGTCCAGCGTTGTGACAGTGCCCAGCAGCTCTCT |
| | | GGGCACCCAGACCTACATCTGCAATGTGAACCACAAGCCTAGCAACA |
| | | CCAAGGTGGACAAGAAGGTGGAACCCAAGAGCTGCGATAAGACACA |
| | | CACCTGTCCTCCATGTCCTGCTCCAGAGCTGCTCGGAGGCCCTTCC |
| | | GTGTTTCTGTTCCCTCCAAAGCCTAAGGACACCCTGATGATCAGCAG |
| | | AACCCCTGAAGTGACCTGCGTGGTGGTGGATGTGTCCCACGAGGAT |
| | | CCCGAAGTGAAGTTCAATTGGTACGTCGACGGCGTGGAAGTGCACA |
| | | ATGCCAAGACCAAGCCTTGCGAGGAACAGTACGGCAGCACCTACAG |
| | | ATGCGTGTCCGTGCTGACAGTGCTGCACCAGGATTGGCTGAACGGC |
| | | AAAGAGTACAAGTGCAAGGTGTCCAACAAGGCCCTGCCTGCTCCTAT |
| | | CGAGAAAACCATCAGCAAGGCCAAGGGCCAGCCTAGAGAACCCCAG |
| | | GTGTACACACTGCCTCCAAGCCGGAAAGAGATGACCAAGAATCAGGT |
| | | GTCCCTGACCTGCCTGGTCAAGGGCTTCTACCCTTCCGATATCGCCG |
| | | TGGAATGGGAGAGCAATGGACAGCCCGAGAACAACTACAAGACAAC |
| | | CCCTCCTGTGCTGAAGTCCGACGGCTCATTCTTCCTGTACAGCAAGC |
| | | TGACCGTGGACAAGAGCAGATGGCAGCAGGGCAACGTGTTCAGCTG |
| | | CAGCGTGATGCACGAGGCCCTGCACAACCACTACACCCAGAAGTCC |
| | | CTGTCTCTGAGCCCCGGCAAA |
| 144 | Complex-67 1^{st} | VSTTCWWDPPCTPNTGSSGGSGGSGGLSGRSDDHGGGSEVQLVESG |
| | polypeptide | GGLVQPGGSLKLSCAASGFTFNKYAMNWVRQAPGKGLEWVARIRSKY |
| | without the | NNYATYYADSVKDRFTISRDDSKNTAYLQMNNLKTEDTAVYYCVRHGNF |
| | spacer and | GNSYISYWAYWGQGTLVTVSSGGGGSGGGGSGGGGSQTVVTQEPSL |
| | without a | TVSPGGTVTLTCGSSTGAVTSGNYPNWVQQKPGQAPRGLIGGTKFLAP |
| | terminal lysine | GTPARFSGSLLGGKAALTLSGVQPEDEAEYYCVLWYSNRWVFGGGTKL |
| | | TVLGGGGSQVQLKQSGPGLVQPSQSLSITCTVSGFSLTNYGVHWVRQS |
| | | PGKGLEWLGVIWSGGNTDYNTPFTSRLSINKDNSKSQVFFKMNSLQSQ |
| | | DTAIYYCARALTYYDYEFAYWGQGTLVTVSAASTKGPSVFPLAPSSKST |
| | | SGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVDGV |
| | | EVHNAKTKPCEEQYGSTYRCVSVLTVLHQDWLNGKEYKCKVSNKALPA |
| | | PIEKTISKAKGQPREPQVYTLPPSRKEMTKNQVSLTCLVKGFYPSDIAVE |
| | | WESNGQPENNYKTTPPVLKSDGSFFLYSKLTVDKSRWQQGNVFSCSV |
| | | MHEALHNHYTQKSLSLSPG |
| 145 | Complex-57 | GYLWGCEWNCGGITTGSSGGSGGSGGLSGRSDDHGGGSQTVVTQEP |
| | 1^{st} Polypeptide | SFSVSPGGTVTLTCRSSTGAVTTSNYANWVQQTPGQAPRGLIGGTNKR |
| | without the | APGVPDRFSGSILGNKAALTITGAQADDESDYYCALWYSNLWVFGGGT |
| | spacer and | KLTVLGGGGSGGGGSGGGGSEVQLVESGGGLVQPGGSLKLSCAASGF |
| | without a | TFSTYAMNWVRQASGKGLEWVGRIRSKYNNYATYYADSVKDRFTISRD |
| | terminal lysine | DSKNTAYLQMNSLKTEDTAVYYCTRHGNFGNSYVSWFAYWGQGTLVT |
| | | VSSGGGGSQVQLKQSGPGLVQPSQSLSITCTVSGFSLTNYGVHWVRQ |
| | | SPGKGLEWLGVIWSGGNTDYNTPFTSRLSINKDNSKSQVFFKMNSLQS |
| | | QDTAIYYCARALTYYDYEFAYWGQGTLVTVSAASTKGPSVFPLAPSSKS |
| | | TSGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYVDG |
| | | VEVHNAKTKPCEEQYGSTYRCVSVLTVLHQDWLNGKEYKCKVSNKALP |
| | | APIEKTISKAKGQPREPQVYTLPPSRKEMTKNQVSLTCLVKGFYPSDIAV |
| | | EWESNGQPENNYKTTPPVLKSDGSFFLYSKLTVDKSRWQQGNVFSCS |
| | | VMHEALHNHYTQKSLSLSPG |
| 146 | CM | ALAHGLF |
| 147 | CM | APRSALAHGLF |
| 148 | CM | ISSGLLSGRSNI |
| 149 | CM | LSGRSNI |

The disclosure is not to be limited in scope by the aspects described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Some aspects are within the following claims.

What is claimed is:

1. An activatable anti-EGFR, anti-CD3 heteromultimeric bispecific polypeptide complex (HBPC) comprising:
(a) a first polypeptide comprising (i) a single-chain variable fragment (scFv) comprising a first heavy chain variable domain (VH1) and a first light chain variable domain (VL1), wherein the VH1 and the VL1 together form a T-cell cluster of differentiation (CD3)-targeting domain that specifically binds a CD3 polypeptide, (ii) a first masking moiety (MM1), (iii) a first cleavable moiety (CM1), (iv) a second heavy chain variable domain (VH2), and (v) a first monomeric Fc domain (Fc1);
(b) a second polypeptide comprising (i) a second light chain variable domain (VL2), wherein the VH2 and the VL2 together form an EGFR targeting domain that specifically binds EGFR, (ii) a second masking moiety (MM2), and (iii) a second cleavable moiety (CM2); and
(c) a third polypeptide that (i) comprises a second monomeric Fc domain (Fc2), and (ii) does not comprise an immunoglobulin variable domain.

2. The activatable bispecific polypeptide complex of claim 1, wherein the CD3 polypeptide is the epsilon chain of CD3.

3. The activatable bispecific polypeptide complex of claim 1, wherein the VH1 comprises:
(i) a VH CDR1 comprising the amino acid sequence KYAMN (SEQ ID NO:3),
(ii) a VH CDR2 comprising the amino acid sequence RIRSKYNNYATYYADSVKD (SEQ ID NO:4), and
(iii) a VH CDR3 comprising the amino acid sequence HGNFGNSYISYWAY (SEQ ID NO:5); and wherein the VL1 comprises:
(i) a VL CDR1 comprising the amino acid sequence GSSTGAVTSGNYPN (SEQ ID NO:6),
(ii) a VL CDR2 comprising the amino acid sequence GTKFLAP (SEQ ID NO:7), and
(iii) a VL CDR3 comprising the amino acid sequence VLWYSNRWV (SEQ ID NO:8).

4. The activatable bispecific polypeptide complex of claim 3, wherein the scFv comprises a VH1 that has an amino acid sequence that is at least 90% identical to SEQ ID NO:9 and/or a VL1 that has an amino acid sequence that is at least 90% identical to SEQ ID NO:10.

5. The activatable bispecific polypeptide complex of claim 4, wherein the scFv comprises a VH1 that has an amino acid sequence of SEQ ID NO:9 and a VL1 that has the amino acid sequence of SEQ ID NO:10.

6. The activatable bispecific polypeptide complex of any one of claims 1-5, wherein the VH2 comprises:
(i) a VH CDR1 comprising the amino acid sequence NYGVH (SEQ ID NO:15),
(ii) a VH CDR2 comprising the amino acid sequence VIWSGGNTDYNTPFTS (SEQ ID NO:16), and
(iii) a VH CDR3 comprising the amino acid sequence ALTYYDYEFAY (SEQ ID NO:17).

7. The activatable bispecific polypeptide complex of any one of claims 1-6, wherein the VL2 comprises:
(i) a VL CDR1 comprising RASQSIGTNIH (SEQ ID NO:18),
(ii) a VL CDR2 comprising YASESIS (SEQ ID NO:19), and
(iii) a VL CDR3 comprising QQNNNWPTT (SEQ ID NO:20).

8. The activatable bispecific polypeptide complex of claim 6, wherein the VH2 comprises an amino acid sequence that is at least 90% identical to SEQ ID NO:21.

9. The activatable bispecific polypeptide complex of claim 6, wherein the VH2 comprises an amino acid sequence of SEQ ID NO:21.

10. The activatable bispecific polypeptide complex of any one of claims 1-9, wherein the Fc1 comprises an amino acid sequence that is at least 90% identical to SEQ ID NO:23.

11. The activatable bispecific polypeptide complex of claim 10, wherein Fc1 comprises the amino acid sequence of SEQ ID NO:23.

12. The activatable bispecific polypeptide complex of any one of claims 1-11, wherein the first polypeptide further comprises a heavy chain CH1 domain between the VH2 and the Fc1.

13. The activatable bispecific polypeptide complex of any one of claims 1-12, wherein the first polypeptide further comprises an immunoglobulin hinge region between the VH2 and the Fc1.

14. The activatable bispecific polypeptide complex of any one of claims 1-13, wherein the first polypeptide comprises a structural arrangement from amino-terminus to carboxy-terminus of: MM1-CM1-scFv-VH2-CH1-hinge region-Fc1, wherein each “-” is independently a direct or indirect linkage.

15. The activatable bispecific polypeptide complex of any one of claims 1-14, wherein the first polypeptide comprises one or more linkers.

16. The activatable bispecific polypeptide complex of claim 15, wherein the linker comprises from about 1 to about 20 amino acids.

17. The activatable bispecific polypeptide complex of any one of claims 1-16, wherein the VL2 comprising:
(i) a VL CDR1 comprising the amino acid sequence RASQSIGTNIH (SEQ ID NO:18),
(ii) a VL CDR2 comprising the amino acid sequence YASESIS (SEQ ID NO:19), and
(iii) a VL CDR3 comprising the amino acid sequence QQNNNWPTT (SEQ ID NO:20).

18. The activatable bispecific polypeptide complex of claim 17, wherein the VL2 comprises an amino acid sequence that is at least 90% identical to SEQ ID NO:22.

19. The activatable bispecific polypeptide complex of claim 18, wherein the VL2 comprises the amino acid sequence of SEQ ID NO:22.

20. The activatable bispecific polypeptide complex of any one of claims 1-19, wherein the second polypeptide comprises a structural arrangement from amino-terminus to carboxy-terminus of: MM2-CM2-VL2, wherein each “-” is independently a direct or indirect linkage.

21. The activatable bispecific polypeptide complex of any one of claims 1-20, wherein the second polypeptide comprises one or more linkers.

22. The activatable bispecific polypeptide complex of claim 21, wherein the linker comprises between about 1 and about 20 amino acids.

23. The activatable bispecific polypeptide complex of any one of claims 1-22, wherein the Fc2 binds to the Fc1.

24. The activatable bispecific polypeptide complex of any one of claims 1-23, wherein the Fc2 comprises an amino acid sequence that is at least 90% identical to SEQ ID NO:28.

25. The activatable bispecific polypeptide complex of claim 24, wherein the Fc2 comprises the amino acid sequence of SEQ ID NO:28.

26. The activatable bispecific polypeptide complex of any one of claims 1-25, wherein at least one of the first polypeptide and the third polypeptide further comprises an immunoglobulin hinge region.

27. The activatable bispecific polypeptide complex of claim 26, wherein each of the first polypeptide and the third polypeptide comprises an immunoglobulin hinge region.

28. The activatable bispecific polypeptide complex of claim 27, wherein the immunoglobulin hinge region of the first polypeptide and immunoglobulin hinge region of the third polypeptide comprises the same amino acid sequence.

29. The activatable bispecific polypeptide complex of claim 27, wherein the immunoglobulin hinge region of the first polypeptide and immunoglobulin hinge region of the third polypeptide comprise different amino acid sequences.

30. The activatable bispecific polypeptide complex of any one of claims 1-29, wherein the third polypeptide comprises an immunoglobulin hinge region in a structural arrangement from amino-terminus to carboxy-terminus of: hinge region-Fc2.

31. The activatable bispecific polypeptide complex of any one of claims 1-29, wherein the first, second, and/or third polypeptide comprise one or more linkers.

32. The activatable bispecific polypeptide complex of any one of claims 1-31, wherein MM1 is linked to CM1 via a linker L1.

33. The activatable bispecific polypeptide complex of any one of claims 1-32, wherein MM2 is linked to CM2 via a linker L2.

34. The activatable bispecific polypeptide complex of any one of claims 1-31, wherein the amino acid sequence of L1 and L2 are the same.

35. The activatable bispecific polypeptide complex of any one of claims 1-31, wherein the amino acid sequence of L1 and L2 are different.

36. The activatable bispecific polypeptide complex of any one of claims 1-35, wherein the CM1 and the CM2 each comprise a substrate for a protease that is present in a tumor microenvironment of a subject having cancer.

37. The activatable bispecific polypeptide complex of any one of claims 1-36, wherein the CM1 and the CM2 each comprise a substrate for the same protease.

38. The activatable bispecific polypeptide complex of any one of claims 1-36, wherein the CM1 and the CM2 comprise substrates for different proteases.

39. The activatable bispecific polypeptide complex of any one of claims 32-38, wherein CM1 and CM2 each independently comprise a substrate for a protease selected from the group of proteases shown in Table 2.

40. The activatable bispecific polypeptide complex of any one of claims 1-39, wherein at least one of the CM1 and CM2 comprises a substrate for a serine protease or matrix metallopeptidase (MMP).

41. The activatable bispecific polypeptide complex of any one of claims 1-40, wherein CM1 comprises the amino acid sequence SEQ ID NO:2 and/or CM2 comprises the amino acid sequence SEQ ID NO:14.

42. The activatable bispecific polypeptide complex of claim 41, wherein CM1 comprises the amino acid sequence of SEQ ID NO:2.

43. The activatable bispecific polypeptide complex of any one of claim 41 or 42, wherein CM2 comprises the amino acid sequence of SEQ ID NO:14.

44. The activatable bispecific polypeptide complex of any one of claims 1-40, wherein CM1 comprises the amino acid sequence SEQ ID NO:73 and/or CM2 comprises the amino acid sequence SEQ ID NO:14.

45. The activatable bispecific polypeptide complex of claim 44, wherein CM1 comprises the amino acid sequence of SEQ ID NO:73.

46. The activatable bispecific polypeptide complex of any one of claim 44 or 45, wherein CM2 comprises the amino acid sequence of SEQ ID NO:14.

47. The activatable bispecific polypeptide complex of any one of claims 1-46, wherein the MM1 and/or the MM2 comprises between about 5 amino acids to about 40 amino acids.

48. The activatable bispecific polypeptide complex of any one of claims 1-46, wherein the MM1 is selected from the group consisting of SEQ ID NO:1, SEQ ID NO:67, SEQ ID NO:68, SEQ ID NO:69, SEQ ID NO:70, SEQ ID NO:71, or SEQ ID NO:72.

49. The activatable bispecific polypeptide complex of any one of claims 1-48, wherein MM2 comprises the amino acid sequences of SEQ ID NO:13.

50. The activatable bispecific polypeptide complex of any one of claims 1-49, wherein MM1 comprises the amino acid sequence of SEQ ID NO:1.

51. The activatable bispecific polypeptide complex of any one of claims 15-50, wherein at least one of the one or more linkers is selected from the group consisting of:
(i) a glycine-serine-based linker selected from the group consisting of (GS)ₙ, wherein n is an integer of at least 1, (GGS)ₙ, wherein n is an integer of at least 1 (e.g., an integer from about 1 to about 20, or from about 1 to about 10), (GGGS)ₙ (SEQ ID NO:40), wherein n is an integer of at least 1 (e.g., an integer from about 1 to about 20, or from about 1 to about 10), (GGGGS)n (SEQ ID NO:126), where n is an integer of at least 1 (e.g., an integer from about 1 to about 20, or from about 1 to about 10), (GSGGS)n (SEQ ID NO:41), wherein n is an integer of at least 1 (e.g., an integer from about 1 to about 20, or from about 1 to about 10), GSSGGSGGSG (SEQ ID NO:12), GGSG (SEQ ID NO:42), GGSGG (SEQ ID NO:43), GSGSG (SEQ ID NO:44), GSGGG (SEQ ID NO:45), GGGSG (SEQ ID NO:46), and GSSSG (SEQ ID NO:47), GGGGSGGGGSGGGGSGS (SEQ ID NO:48), GGGGSGS (SEQ ID NO:49), GGGGSGGGGSGGGGS (SEQ ID NO:50), GGGGSGGGGSGGGGSGGGGS (SEQ ID NO:51), GGGGS (SEQ ID NO:52), GGGGSGGGGS (SEQ ID NO:53), GGGS (SEQ ID NO:54), GGGSGGGS (SEQ ID NO:55), GGGSGGGSGGGS (SEQ ID NO:56), GSSGGSGGSGG (SEQ ID NO:57), GGGSGGGGSGGGGSGGGGSGGGGS (SEQ ID NO:58), GGGSSGGS (SEQ ID NO:127) and GS; and
(ii) a linker comprising glycine and serine, and at least one of lysine, threonine, or proline selected from the group consisting of GSTSGSGKPGSSEGST (SEQ ID NO:59), SKYGPPCPPCPAPEFLG (SEQ ID NO:60), GGSLDPKGGGGS (SEQ ID NO:61), PKSCDKTHTCPPCPAPELLG (SEQ ID NO:62), GKSSGSGSESKS (SEQ ID NO:63), GSTSGSGKSSEGKG (SEQ ID NO:64), GSTSGSGKSSEGSGSTKG (SEQ ID NO:65), and GSTSGSGKPGSGEGSTKG (SEQ ID NO:66).

52. The activatable bispecific polypeptide complex of any one of claims 1-51, wherein: (1) the first polypeptide comprises the amino acid sequence of SEQ ID NO:30, (2) the second polypeptide comprises the amino acid sequence of SEQ ID NO:31, and (3) the third polypeptide comprises the amino acid sequence of SEQ ID NO:32.

53. The activatable bispecific polypeptide complex of any one of claims 1-51, wherein: (1) the first polypeptide comprises the amino acid sequence of SEQ ID NO:120, (2) the second polypeptide comprises the amino acid sequence of SEQ ID NO:37, and (3) the third polypeptide comprises the amino acid sequence of SEQ ID NO:32.

54. The activatable bispecific polypeptide complex of any one of claims 1-51, wherein: (1) the first polypeptide comprises the amino acid sequence of SEQ ID NO:144, (2) the second polypeptide comprises the amino acid sequence of SEQ ID NO:37, and (3) the third polypeptide comprises the amino acid sequence of SEQ ID NO:32.

55. A pharmaceutical composition comprising the activatable bispecific polypeptide complex of any one of claims 1-54 and a pharmaceutically acceptable carrier.

56. A kit comprising the pharmaceutical composition of claim 55.

57. A nucleic acid comprising nucleotide sequences that encode the first polypeptide, the second polypeptide, and the third polypeptide of the activatable bispecific polypeptide of any one of claims 1-54.

58. A vector comprising the nucleic acid of claim 57.

59. A host cell comprising the vector of claim 58.

60. A method of producing an activatable heteromultimeric bispecific polypeptide complex (HBPC) comprising:
(a) culturing the host cell of claim 59 in a liquid culture medium under conditions sufficient to produce the HBPC; and
(b) recovering the HBPC.

61. A method of treating a disease in a subject comprising administering a therapeutically effective amount of the activatable bispecific polypeptide complex of any one of claims 1-54 or the pharmaceutical composition of claim 55 to the subject.

62. The method of claim 61, wherein the subject is a human.

63. The method of claim 61 or 62, wherein the disease is a cancer.

64. The activatable bispecific polypeptide of any one of claims 1-54 or the pharmaceutical composition of claim 55 for use in inhibiting tumor growth in a subject in need thereof.

65. Use of an activatable bispecific polypeptide complex according to any one of claims 1-54 or the pharmaceutical composition of claim 55 in the manufacture of a medicament for treating cancer.
